(19) United States
(12) Patent Application Publication
(10) Pub. No.: US 2024/0189376 A1
(43) Pub. Date: Jun. 13, 2024

(54) NOVEL MUSHROOM STRAINS, EXTRACTS THEREOF AND COMPOSITIONS COMPRISING THEM

(71) Applicant: CANNABOTECH LTD., Herzliya (IL)

(72) Inventors: Solomon P. WASSER, Nesher (IL); Basem FARES, Horfesh (IL); Gadi KLARSFELD, Hod Hasharon (IL); Dobroslav MELAMED, Rishon Lezion (IL)

(21) Appl. No.: 18/550,737

(22) PCT Filed: Mar. 14, 2023
(86) PCT No.: PCT/IL2022/050284

§ 371 Date: Sep. 15, 2023

Related U.S. Application Data
(60) Provisional application No. 63/161,643, filed on Mar. 16, 2021.
(60) Provisional application No. 63/262,067, filed on Oct. 4, 2021.

(51) Int. Cl. A61K 36/07; A01H 15/00; A61K 8/9728; A61K 31/00; A61K 36/185; A61Q 19/00

(52) U.S. Cl. CPC A61K 36/07; A01H 15/00; A61K 8/9728; A61K 31/658; A61K 36/185; A61Q 19/00; A61K 2236/331; A61K 2800/10; A61K 2800/522

(57) ABSTRACT

The present invention relates to medicinal mushrooms, more specifically to novel strains of Coprinus comatus, Flammulina velutipes, Ganoderma lucidum, Grifola frondosa, Hericium erinaceus, Pleurotus ostreatus, and Trametes versicolor, extracts thereof, and compositions comprising said extracts.

### TECHNICAL FIELD

The present invention relates to medicinal mushrooms, more specifically to novel strains of Coprinus comatus, Flammulina velutipes, Ganoderma lucidum, Grifola frondosa, Hericium erinaceus, Pleurotus ostreatus, and Trametes versicolor, extracts thereof, and compositions comprising said extracts.

### BACKGROUND ART

Medicinal mushrooms (MMs) have a proven history of use worldwide. Nowadays, they are used as dietary food, dietary supplement products, a new class of drugs called “Mushroom Pharmaceuticals”, natural bio-control agents in plant, dietary pet and veterinary food supplements, and cosmeceuticals and nutricosmetics. Different compounds of MMs include polysaccharides such as soluble β-glucans, glucuronoxylomannan, sacchachitin, tyrosinase, and other enzymes (Chang and Wasser, 2018). MMs are capable to produce wide range of bioactive substances with many pharmaceutical prospects, and MM-based products thus have a wide range of uses for the treatment and prevention of many different diseases.

More than 200 medicinal functions are thought to be produced by MMs including antitumor, immunomodulating, antioxidant, radical scavenging, cardiovascular, cholesterol-lowering, antiviral, antibacterial, anti-parasitic, antifungal, detoxification, hepatoprotective, anti-diabetic, anti-obesity, neuroprotective, and neuro-regenerative. In addition, substances derived from MMs can be used as painkillers or analgesics. MM-based drugs and dietary supplements have been implemented in preventing immune disorders and maintaining a good quality of life in immunodeficient and immuno-depressed patients; patients under chemotherapy or radiotherapy; patients having different types of cancers, chronic blood-borne viral infections of Hepatitis B. C, and D, different types of anemia, the human immunodeficiency virus/acquired immunodeficiency syndrome (HIV/AIDS), Herpes simplex virus (HSV), Epstein Bar virus, Influenza viruses A and B. H5N1, COVID-19 (Murphy et al., 2020), West Nile virus, chronic fatigue syndrome, chronic gastritis, or gastric ulcers caused by Helicobacter pylori; and people suffering from dementia (especially Alzheimer's disease) (Wasser, 2010, Chang and Wasser, 2018).

MM have the greatest potential for medical and pharmaceutical use due to their ability to synthesize many highly beneficial bioactive compounds, and could be a great source of, e.g., dietary fiber, polysaccharides (mainly β-glucans) and polysaccharide-protein complexes, and various low-molecular-weight compounds including amino acids, fatty acids, vitamins, triterpens, terpenoids, lectins, steroids, phenols, polyphenols, lactones, statins, alkaloids, and antibiotics (Wasser, 2010; Cohen et al., 2014; Wasser 2017, 2018; Chang and Wasser, 2018; Watanabe and Bito, 2018; Xue et al., 2020; Morales et al., 2020; Mehra et al., 2020).

Among the different bioactive compounds, special attention is paid to polysaccharides and phenolic compounds. According to the art, polysaccharides and polysaccharide-protein complexes from MMs enhance innate and cell-mediated immune responses and exhibit antitumor activities. Mushroom polysaccharides prevent oncogenesis, show direct antitumor activity against various synergetic tumors, and prevent tumor metastasis. Their activity is especially beneficial when used in conjunction with chemotherapy. The antitumor action of polysaccharides requires an intact T-cell component; their activity is mediated through a thymus-dependent immune mechanism. They activate cytotoxic macrophages, monocytes, neutrophils, natural killer cells, dendritic cells, and chemical messengers (cytokines, such as interleukins, interferons, and colony stimulating factors) that trigger complementary and acute phase responses. Also, mushroom polysaccharides can be considered as multi-cytokine inducers able to induce gene expression of various immunomodulating cytokines, and cytokine receptors.

New class of drugs were developed from MMs called “mushroom pharmaceuticals/drugs”, or biological response modifiers like krestin (PSK) and PSP (polysaccharide peptide) from Trametes versicolor; lentinan, isolated from Lentinus edodes; schizophyllan (sonifilan, sizofiran, or SPG) from Schizophyllum commune; befungin from Inonotus obliquus; D-fraction from Grifola frondosa, GLPS polysaccharide fraction from Ganoderma lucidum; active hexose correlated compound (AHCC), and some others (Wasser, 2010, Chang and Wasser, 2018).

Several of the mushroom compounds have been tested in Phase I-IV clinical trials and are used extensively and successfully in Asia to treat various cancers and other diseases. Approximately 300 clinical studies were conducted only on Ganoderma lucidum and some other species of genus Ganoderma. Most of the clinical trials were done mainly using G. lucidum, Lentinus edodes, Grifola frondosa, Trametes versicolor, Schizophyllum commune, Phellinus linteus, and Agaricus brasiliensis for treatment of cancers, oncoimmunological, and immunological diseases and in immune-adjuvant therapy. Fruiting bodies of mushrooms and/or their biomass from submerged cultivated mycelia, different types of extracts, rare spores (from G. lucidum), and pure ß-glucans (e.g., lentinan or schizophyllan isolated from cultural broth), or proteoglucan (PSK) or polysaccharide peptide (PSP) have been used in clinical trials for cancer treatment. In many cases, mushrooms were used as adjuvant treatment with conventional chemo- or radiotherapy in different kinds of cancer (Wasser, 2017, Vetvicka et al., 2019). Beside this, it should be noted that phenolic compounds exist in all tested mushroom species. It has been reported that different types of phenolic compounds act as effective antioxidants in biological systems, acting as free radical inhibitors, peroxide decomposers, or oxygen scavengers (Sanchez, 2017). In recent years it became known that synthetic antioxidants such as butylhydroxyanisole, butylhydroxytoluene, and tert-butylhydroquinone have a toxic effect (especially in high concentrations) and could be responsible for different types of tumor and liver damage.

The revealing of new, effective, natural antioxidants, which do not induce toxic effects, is a significant challenge. Both, polysaccharides and phenolic compounds have been documented as antioxidants, and mushrooms are thus considered to be an excessive source of natural antioxidants having pharmaceutical potential.

The prevalence of mushroom dietary supplement use has increased dramatically over the past 20 years. Dietary supplements are often used to reduce side effects and toxicity caused by the treatment of various diseases (mainly cancer), stimulate and protect immunity, or prevent other complications following treatment. Western and Eastern medicine has adopted different regulatory systems for mushroom preparations. Most Western countries follow the rules of the World Health Organization (WHO) and the Dietary Supplements Health and Education Act (DSHEA, 1994) according to which medicinal mushroom extracts are defined as dietary supplements and clinical studies are not required before introducing to the market.

### SUMMARY OF INVENTION

In one aspect, the present invention relates to a new and distinct variety of higher Basidiomycetes mushroom, e.g., grown in submerged cultures, selected from:
(i) Coprinus comatus (O.F. Müll.) Pers. deposited under the Budapest Treaty with the Centraalbureau voor Schimmelcultures (CBS) under Accession No. CBS 146994 (hereinafter Coprinus comatus CBS 146994, or CNBT-234);
(ii) Flammulina velutipes (Curtis) Singer deposited under the Budapest Treaty with the CBS under Accession No. CBS 146995 (hereinafter Flammulina velutipes CBS 146995, or CNBT-94);
(iii) Ganoderma lucidum (Curtis: Fr.) P. Karst. deposited under the Budapest Treaty with the CBS under Accession No. CBS 146996 (hereinafter Ganoderma lucidum CBS 146996, or CNBT-137);
(iv) Grifola frondosa (Dicks.) Gray deposited under the Budapest Treaty with the CBS under Accession No. CBS 146997 (hereinafter Grifola frondosa CBS 146997, or CNBT-118);
(v) Hericium erinaceus (Bull.: Fr.) Pers. deposited under the Budapest Treaty with the CBS under Accession No. CBS 146998 (hereinafter Hericium erinaceus CBS 146998, or CNBT-153);
(vi) Pleurotus ostreatus (Jacq.: Fr.) Kummer deposited under the Budapest Treaty with the CBS under Accession No. CBS 146999 (hereinafter Pleurotus ostreatus CBS 146999, or CNBT-27); and
(vii) Trametes versicolor (L.: Fr.) Lloyd. deposited under the Budapest Treaty with the CBS under Accession No. CBS 147000 (hereinafter Trametes versicolor CBS 147000, or CNBT-112).

In another aspect, the present invention relates to a biomass of a higher Basidiomycetes mushroom as defined above, e.g., in the form of a powder.

In a further aspect, the present invention provides an extract obtained from a higher Basidiomycetes mushroom or a combination thereof, wherein at least one of said higher Basidiomycetes mushrooms is the higher Basidiomycetes mushroom defined above. Particular such extracts are obtained from a combination of higher Basidiomycetes mushrooms, wherein at least one of said mushrooms is a higher Basidiomycetes mushroom as defined above, and at least another one of said mushrooms is selected from the species Agaricus brasiliensis, Auricularia aricula-judae, Cordyceps militaris, Ganoderma tsugae, Hypsizygus marmoreus, Inonotus obliquus, Lentinus edodes, Pleurotus eryngii, and Tremella fuciformis. The extract disclosed herein may be obtained by extracting said higher Basidiomycetes mushroom or combination thereof with an organic or inorganic solvent. Particular such extracts are obtained by extracting said mushroom or combination thereof with water at a temperature of about 60-90° ° C., about 70-85° C., or about 80° C., or with an organic solvent such as an alcohol (e.g., ethanol).

In yet another aspect, the present invention provides a composition comprising an extract as defined above, or a combination thereof, e.g., a combination of (i) an extract obtained by extracting one or more higher Basidiomycetes mushrooms with water; and (ii) an extract obtained by extracting one or more higher Basidiomycetes mushrooms with an alcohol. Such a composition may further comprise an active agent such as a cannabinoid, a combination thereof, or a Cannabis plant extract comprising said cannabinoid or combination thereof. In particular such composition, said active agent is cannabidiol (CBD) or an enantiomer, diastereomer, or a mixture thereof, preferably CBD.

The composition disclosed herein, i.e., those comprising solely said extract or combination thereof, and those further comprising an active agent, may be a nutraceutical composition optionally further comprising a nutraceutically acceptable carrier, or a cosmetic composition optionally further comprising a cosmetically acceptable carrier.

In still another aspect, the present invention relates to a drink, beverage, or a food (diatery) supplement, comprising a composition as disclosed herein.

In yet a further aspect, the present invention relates to a method of providing amino acids, phenolic compounds, flavonoid compounds, and/or antioxidant substances to a subject, said method comprising administering to said subject a nutraceutical composition as defined above.

In still a further aspect, the present invention relates to use of a nutraceutical composition as defined above as a dietary supplement rich in amino acids, phenolic compounds, flavonoid compounds, and/or antioxidant substances.

### BRIEF DESCRIPTION OF DRAWINGS

FIG. 1 shows cytotoxicity analysis as assessed by LDH from RAW264.7 macrophages. LDH levels were measured by OD (arbitrary units) and are presented as mean±SD. At least eight replicates were in each group (n=8 for naïve, n=16 for vehicle, and n=14 for the Immunity formula). ANOVA with Bonferroni's post-hoc test was performed. p=0.92.

FIGS. 2A-2C show the effect of the Immunity formula on the release of pro-inflammatory cytokines induced by LPS in RAW264.7 macrophages. RAW 264.7 macrophages were exposed to 20 ng/ml LPS and simultaneously to the Immunity formula (20 μg/ml). IL-1B (2A), IL-6 (2B), and TNF-α (2C) levels were measured using ELISA. The results are presented as mean±SD (n=4 in each group). ANOVA followed by Bonferroni's post-hoc test was performed. *** p<0.001 compared to all other groups.

FIG. 3 shows the effect of the Immunity formula on the release of CCL5 in RAW264.7 macrophages following LPS stimulation. CCL5 levels were measured (in pg/ml) using standard calibration curve and are presented as mean±SD (n=4 in each group). ANOVA followed by Bonferroni's post-hoc test was performed. *** p<0.001 compared to all other groups.

FIG. 4 shows the effect of Immunity formula on COX-2 protein expression in RAW264.7 macrophages following LPS stimulation. COX-2 levels were measured by ELISA using standard calibration curve and are presented as mean±SD (n=4 in each group). ANOVA followed by Bonferroni's post-hoc test was performed. *** p<0.001 compared to all other groups.

FIG. 5 shows nitrite concentration in RAW264.7 macrophages following LPS stimulation. Nitrite levels were measured using standard calibration curve with sodium nitrite and are presented as mean±SD (n=4 in each group). ANOVA followed by Bonferroni's post-hoc test was performed. *** p<0.001 compared to all other groups.

### DETAILED DESCRIPTION

In one aspect, the present invention relates to a new and distinct variety of higher Basidiomycetes mushroom, e.g., grown in submerged cultures, selected from Coprinus comatus CBS 146994, Flammulina velutipes CBS 146995, Ganoderma lucidum CBS 146996, Grifola frondosa CBS 146997, Hericium erinaceus CBS 146998, Pleurotus ostreatus CBS 146999, and Trametes versicolor CBS 147000.

In another aspect, the present invention relates to a biomass of a higher Basidiomycetes mushroom as disclosed herein, i.e., Coprinus comatus CBS 146994, Flammulina velutipes CBS 146995, Ganoderma lucidum CBS 146996, Grifola frondosa CBS 146997. Hericium erinaceus CBS 146998, Pleurotus ostreatus CBS 146999, or Trametes versicolor CBS 147000. The term “biomass” as used herein with respect to said higher Basidiomycetes mushroom refers to a biomass of said mushroom, or of a part thereof, e.g., fruiting bodies and mycelium. In certain embodiments, the biomass disclosed is in the form of a powder.

In a further aspect, the present invention provides an extract obtained from a higher Basidiomycetes mushroom or a combination thereof, wherein at least one of said higher Basidiomycetes mushrooms is a higher Basidiomycetes mushroom as disclosed herein, i.e., Coprinus comatus CBS 146994, Flammulina velutipes CBS 146995, Ganoderma lucidum CBS 146996, Grifola frondosa CBS 146997, Hericium erinaceus CBS 146998, Pleurotus ostreatus CBS 146999, or Trametes versicolor CBS 147000. In certain embodiments, said extract is obtained from a combination of higher Basidiomycetes mushrooms, wherein at least one of said mushrooms is a higher Basidiomycetes mushroom as disclosed herein, and at least another one of said mushrooms is selected from the species Agaricus brasiliensis, e.g., the strain Agaricus brasiliensis Wasser et al. exemplified herein, Auricularia aricula-judae, e.g., the strain Auricularia aricula-judae (Bull.) J. Schröt. exemplified herein, Cordyceps militaris, e.g., the strain Cordyceps militaris (L.) Link exemplified herein, Ganoderma tsugae, e.g., the strain Ganoderma tsugae Murrill exemplified herein, Hypsizygus marmoreus, e.g., the strain Hypsizygus marmoreus (Peck) H. E. Bigelow exemplified herein, Inonotus obliquus, e.g., the strain Inonotus obliquus (Ach. ex Pers.) Pilát exemplified herein, Lentinus edodes, e.g., the strain Lentinus edodes (Berk.) Singer exemplified herein, Pleurotus eryngii, e.g., the strain Pleurotus eryngii (DC: Fr.) Quél. exemplified herein, and Tremella fuciformis, e.g., the strain Tremella fuciformis Berk. exemplified herein.

Extracts according to the invention may be obtained by extracting said higher Basidiomycetes mushroom, a part (e.g., the fruiting bodies or mycelium) thereof, or a combination thereof with an organic or inorganic solvent. Particular such extracts are obtained by extracting said mushroom, part thereof, or combination thereof with water, e.g., distilled water, at a temperature of about 60-90° C., 70-85° C., or 80° C., and are rich in, e.g., polysaccharides, proteins, and amino acids, in particular essential amino acids. Other such extracts are obtained by extracting said mushroom, part thereof, or combination thereof with an organic solvent such as methanol, ethanol, isopropanol, hexanol, heptane, cyclohexane, methylcyclohexane, dichloromethane, acetonitrile, acetone, methyl ethyl ketone, diethyl ether, methyl-tert-butyl ether (MTBE), chloroform, tetrahydrofuran (THF), dioxane, phenol, or a mixture thereof.

The term “amino acid” as used herein with respect to the extracts of the invention refers to any natural amino acid including the twenty-two amino acids naturally occurring in proteins, i.e., aspartic acid, tyrosine, leucine, tryptophan, arginine, valine, glutamic acid, methionine, phenylalanine, serine, alanine, glutamine, glycine, proline, threonine, asparagine, lysine, histidine, isoleucine, cysteine, selenocysteine, and pyrrolysine (of which alanine, arginine, aspartic acid, glutamine, isoleucine, leucine, lysine, methionine, phenylalanine, serine, and threonine are considered essential amino acids), as well as to γ-aminobutyric acid (GABA) and ergothioneine. Analysis of the amino acids in the extracts of the invention may be carried out using any suitable method or technique known in the art, e.g., as described in Examples 6-7 herein.

The term “protein” as used herein with respect to the extracts of the invention refers to protein of mushroom origin (which have comparatively high thermal and pH stability), that have high nutritional values associated with the rich level of essential amino acids. The crude protein may contain lectins, antifungal proteins, ubiquitin-like, and ribonucleases proteins and peptides, fungal immunomodulatory protein, and enzymes. Analysis of the protein content may be carried out using any method or technique known in the art, e.g., as described in Example 5 herein.

Polysaccharides are long chain polymeric carbohydrates composed of monosaccharide units bound together by glycosidic linkages, which may range in structure from linear to highly branched, and undergo hydrolysis (in the presence of amylase enzymes as catalysts) to provide constituent sugars (monosaccharides or oligosaccharides). The term “polysaccharides” as used herein with respect to the extracts of the invention mainly refers to such branched polymeric carbohydrates consisting of D-glucose residue backbone with various chains of β-1,3 residues such as, but not limited to, mannose, glucose, xylose, and glucuronic acid. Determination of the polysaccharides content may be carried out using any method or technique known in the art, e.g., as described in Example 5 herein.

The term “triterpene” as used herein with respect to the extracts of the invention refer to a chemical compound composed of three terpene units with the molecular formula C₃₀H₄₈. Triterpenoids are triterpenes that possess heteroatoms, usually oxygen.

In certain specific embodiments, the extract disclosed herein is obtained from a combination of Coprinus comatus CBS 146994, Lentinus edodes, e.g., Lentinus edodes (Berk.) Singer, and Tremella fuciformis, e.g., Tremella fuciformis Berk., e.g., at a weight ratio of about 1:1:1 (referred to herein as “Extract 1”), by extracting said combination with water at a temperature of about 60-90° C. As shown herein, Extract 1 may contain about 10-18%, e.g., about 12-16%, total polysaccharides, about 4-6%, e.g., about 4.5-5%, proteins, about 0.6-0.7%, e.g., about 0.62-0.68%, fats, and/or total energy value of about 22-28, e.g., about 24-26, calories/100 g; and/or is rich in alanine, arginine, glutamine, isoleucine, lysine, methionine, phenylalanine, serine, and threonine.

In certain specific embodiments, the extract disclosed herein is obtained from a combination of Flammulina velutipes CBS 146995, Ganoderma lucidum CBS 146996, Lentinus edodes, e.g., Lentinus edodes (Berk.) Singer, and Trametes versicolor CBS 147000, e.g., at a weight ratio of about 1:1:1:1 (referred to herein as “Extract 2”), by extracting said combination with water at a temperature of about 60-90° C. As shown herein, Extract 2 may contain about 54-64%, e.g., about 56-60%, total polysaccharides, about 1.5-2.5%, e.g., about 1.8-2%, proteins, about 0.8-1.4%, e.g., about 0.9-1.2%, fats, and/or total energy value of about 22-28, e.g., about 24-26, calories/100 g; and/or is rich in alanine, arginine, glutamine, isoleucine, lysine, methionine, phenylalanine, serine, and threonine.

In certain specific embodiments, the extract disclosed herein is obtained from a combination of Cordyceps militaris, e.g., Cordyceps militaris (L.) Link, Ganoderma lucidum CBS 146996, and Inonotus obliquus, e.g., Inonotus obliquus (Ach. ex Pers.) Pilát, e.g., at a weight ratio of about 1:1:2, respectively (referred to herein as “Extract 3”), by extracting said combination with water at a temperature of about 60-90° C. As shown herein, Extract 3 may contain about 13-16%, e.g., about 14-15%, total polysaccharides, about 3.5-4.2%, e.g., about 3.8-4%, proteins, about 0.5-0.7%, e.g., about 0.56-0.62%, fats, and/or total energy value of about 18-24, e.g., about 20-22, calories/100 g; and/or is rich in alanine, arginine, glutamine, isoleucine, leucine, lysine, methionine, phenylalanine, serine, and threonine.

In certain specific embodiments, the extract disclosed herein is obtained from a combination of Grifola frondosa CBS 146997, Lentinus edodes, e.g., Lentinus edodes (Berk.) Singer, Pleurotus eryngii, e.g., Pleurotus eryngii (DC: Fr.) Quél., and Trametes versicolor CBS 147000, e.g., at a weight ratio of about 1:1:1:1 (referred to herein as “Extract 4”), by extracting said combination with water at a temperature of about 60-90° C. As shown herein, Extract 4 may contain about 10-24%, e.g., about 15-20%, total polysaccharides, about 3.5-5%, e.g., about 4-4.5%, proteins, about 0.1-0.2%, e.g., about 0.12-0.16%, fats, and/or total energy value of about 16-20, e.g., about 18-19, calories/100 g; and/or is rich in alanine, arginine, glutamine, isoleucine, leucine, lysine, methionine, phenylalanine, serine, and threonine.

In certain specific embodiments, the extract disclosed herein is obtained from a combination of Auricularia aricula-judae, e.g., Auricularia aricula-judae (Bull.) J. Schröt., Ganoderma lucidum CBS 146996, Lentinus edodes, e.g., Lentinus edodes (Berk.) Singer, Pleurotus ostreatus CBS 146999, and Trametes versicolor CBS 147000, e.g., at a weight ratio of about 1.5:1.5:1.5:4:1.5, respectively (referred to herein as “Extract 5”), by extracting said combination with water at a temperature of about 60-90° C. As shown herein, Extract 5 may contain about 20-34%, e.g., about 24-28%, total polysaccharides, about 3-4%, e.g., about 3.4-3.8%, proteins, about 0.3-0.8%, e.g., about 0.4-0.6%, fats, and/or total energy value of about 17-22, e.g., about 18-20, calories/100 g; and/or is rich in arginine, lysine, methionine, phenylalanine, serine, and threonine.

In certain specific embodiments, the extract disclosed herein is obtained from a combination of Auricularia aricula-judae, e.g., Auricularia aricula-judae (Bull.) J. Schröt., Ganoderma lucidum CBS 146996, Lentinus edodes, e.g., Lentinus edodes (Berk.) Singer, Pleurotus ostreatus CBS 146999, and Trametes versicolor CBS 147000, at a weight ratio of about 2:2:1.5:3:1.5, respectively (referred to herein as “Extract 6”), by extracting said combination with water at a temperature of about 60-90° C. As shown herein, Extract 6 may contain about 15-28%, e.g., about 18-23%, total polysaccharides, about 3-3.8%, e.g., about 3.2-3.5%, proteins, about 0.2-1.2%, e.g., about 0.4-0.8%, fats, and/or total energy value of about 16-22, e.g., about 18-20, calories/100 g; and/or is rich in alanine, arginine, lysine, methionine, phenylalanine, serine, and threonine.

In certain specific embodiments, the extract disclosed herein is obtained from a combination of Flammulina velutipes CBS 146995, Ganoderma lucidum CBS 146996, Hypsizygus marmoreus, e.g., Hypsizygus marmoreus (Peck) H. E. Bigelow, Lentinus edodes, e.g., Lentinus edodes (Berk.) Singer, and Trametes versicolor CBS 147000, e.g., at a weight ratio of about 1:1:1:1:1 (referred to herein as “Extract 7”), by extracting said combination with water at a temperature of about 60-90° C. As shown herein, Extract 7 may contain about 3-6%, e.g., about 4-5%, total polysaccharides, about 3.5-4.2%, e.g., about 3.7-3.9%, proteins, about 0.17-0.19%, e.g., about 0.18%, fats, and/or total energy value of about 15-20, e.g., about 17-18, calories/100 g; and/or is rich in alanine, arginine, leucine, lysine, methionine, phenylalanine, serine, and threonine.

In certain specific embodiments, the extract disclosed herein is obtained from a combination of Cordyceps militaris, e.g., Cordyceps militaris (L.) Link, Ganoderma lucidum CBS 146996, and Inonotus obliquus, e.g., Inonotus obliquus (Ach. ex Pers.) Pilát, e.g., at a weight ratio of about 3:4:3, respectively (referred to herein as “Extract 8”), by extracting said combination with water at a temperature of about 60-90° ° C. As shown herein, Extract 8 may contain about 24-28%, e.g., about 25-27%, total polysaccharides, about 1.8-3%, e.g., about 2.2-2.6%, proteins, about 0.2-0.5%, e.g., about 0.3-0.4%, fats, and/or total energy value of about 10-15, e.g., about 12-14, calories/100 g; and/or is rich in alanine, arginine, aspartic acid, leucine, lysine, methionine, phenylalanine, serine, and threonine.

In certain specific embodiments, the extract disclosed herein is obtained from a combination of Cordyceps militaris, e.g., Cordyceps militaris (L.) Link, Ganoderma lucidum CBS 146996, and Hericium erinaceus CBS 146998, e.g., at a weight ratio of about 3:3:4, respectively (referred to herein as “Extract 9”), by extracting said combination with water at a temperature of about 60-90° C. As shown herein, Extract 9 may contain about 24-30%, e.g., about 26-28%, total polysaccharides, about 3.3-3.7%, e.g., about 3.4-3.6%, proteins, about 0.2-0.5%, e.g., about 0.3-0.4%, fats, and/or total energy value of about 16-20, e.g., about 17-18, calories/100 g; and/or is rich in arginine, aspartic acid, glutamine, leucine, lysine, methionine, phenylalanine, and threonine.

In certain specific embodiments, the extract disclosed herein is obtained from a combination of Cordyceps militaris, e.g., a combination of Cordyceps militaris (L.) Link, Ganoderma lucidum CBS 146996, and Ganoderma tsugae, e.g., Ganoderma tsugue Murrill, e.g., at a weight ratio of about 2:1:1, respectively (referred to herein as “Extract 10”), by extracting said combination with water at a temperature of about 60-90° C. As shown herein, Extract 10 may contain about 22-34%, e.g., about 26-30%, total polysaccharides, about 1.7-2%, e.g., about 1.8-1.9%, proteins, about 0.2-0.4%, e.g., about 0.28-0.34%, fats, and/or total energy value of about 8-12, e.g., about 10-11, calories/100 g; and/or is rich in alanine, arginine, aspartic acid, leucine, lysine, methionine, phenylalanine, serine, and threonine.

In certain specific embodiments, the extract disclosed herein is obtained from a combination of Ganoderma lucidum CBS 146996, and of Hericium erinaceus CBS 146998, e.g., at a weight ratio of about 1:3, respectively (referred to herein as “Extract 11”), by extracting said combination with water at a temperature of about 60-90° C. As shown herein, Extract 11 may contain about 8-18%, e.g., about 12-15%, total polysaccharides, about 1.6-2.6%, e.g., about 2-2.2%, proteins, no fats, and/or total energy value of about 6-10, e.g., about 8-9, calories/100 g; and/or is rich in alanine, arginine, aspartic acid, glutamine, leucine, lysine, methionine, phenylalanine, and threonine.

In certain specific embodiments, the extract disclosed herein is obtained from a combination of Agaricus brasiliensis, e.g., Agaricus brasiliensis Wasser et al., Coprinus comatus CBS 146994, Cordyceps militaris, e.g., Cordyceps militaris (L.) Link, Lentinus edodes, e.g., Lentinus edodes (Berk.) Singer, and Pleurotus ostreatus CBS 146999, e.g., at a weight ratio of about 1.5:1.5:1.5:4:1.5, respectively (referred to herein as “Extract 12”), by extracting said combination with water at a temperature of about 60-90° C. As shown herein, Extract 12 may contain about 25-35%, e.g., about 28-32%, total polysaccharides, about 3.4-4.2%, e.g., about 3.6-4%, proteins, about 0.24-0.28%, e.g., about 0.26%, fats, and/or total energy value of about 13-18, e.g., about 15-16, calories/100 g; and/or is rich only in lysine, methionine, and phenylalanine.

In certain specific embodiments, the extract disclosed herein is obtained from a combination of Agaricus brasiliensis, e.g., Agaricus brasiliensis Wasser et al., Coprinus comatus CBS 146994, Ganoderma lucidum CBS 146996, and Tremella fuciformis, e.g., Tremella fuciformis Berk., e.g., at a weight ratio of about 1:2:1:1, respectively (referred to herein as “Extract 13”), by extracting said combination with water at a temperature of about 60-90° C. As shown herein, Extract 13 may contain about 8-12%, e.g., about 9-11%, total polysaccharides, about 5-6%, e.g., about 5.2-5.6%, proteins, about 0.1-0.3%, e.g., about 0.2%, fats, and/or total energy value of about 22-25, e.g., about 23-24, calories/100 g; and/or is rich in alanine, arginine, isoleucine, leucine, lysine, methionine, phenylalanine, and threonine.

In certain specific embodiments, the extract disclosed herein is obtained from a combination of Agaricus brasiliensis, e.g., Agaricus brasiliensis Wasser et al., Cordyceps militaris, e.g., Cordyceps militaris (L.) Link, Ganoderma lucidum CBS 146996, Grifola frondosa CBS 146997, and Tremella fuciformis, e.g., Tremella fuciformis Berk., e.g., at about equal weights (referred to herein as “Extract 14”), by extracting said combination with water at a temperature of about 60-90° C. As shown herein, Extract 14 may contain about 23-26%, e.g., about 24-25%, total polysaccharides, about 3.5-3.8%, e.g., about 3.6-3.7%, proteins, no fats, and/or total energy value of about 12-16, e.g., about 14-15 calories/100 g; and/or is rich in alanine, arginine, leucine, lysine, methionine, phenylalanine, and threonine.

In certain specific embodiments, the extract disclosed herein is obtained from a combination of Trametes versicolor CBS 147000, Cordyceps militaris, e.g., Cordyceps militaris (L.) Link, Flammulina velutipes CBS 146995, Ganoderma lucidum CBS 146996, and Lentinus edodes, e.g., Lentinus edodes (Berk.) Singer, e.g., at about equal weights (referred to herein as “Extract 15”), by extracting said combination with water at a temperature of about 60-90° ° C. As shown herein, Extract 15 may contain about 28-40%, e.g., about 30-35%, or about 32%, total polysaccharides, as well as certain amino acids and/or antioxidants.

In yet another aspect, the present invention provides a composition comprising an extract as defined in any one of the embodiments above, more particularly an extract obtained by extracting said higher Basidiomycetes mushroom or combination thereof with water, e.g., one of the extracts specifically referred to above including Extracts 1-15, or with an alcohol, or a combination thereof. A particular such composition exemplified herein comprises a combination of the specific extract identified herein as Extract 15 and an extract obtained by extracting Ganoderma lucidum, e.g., Ganoderma lucidum CBS 146996, with ethanol (e.g., 70%), at a ratio of, e.g., about 9:1, respectively, by volume. The benefits of such a combination are the high amount of total polysaccharides in Extract 15 and the relatively high amount of total triterpenes and triterpenoids in the ethanolic extract of Ganoderma lucidum, which may be in a range of about 6% to about 15% or more.

The composition of the present invention, according to any one of the embodiments above, may further comprise an (additional) active agent, wherein the mushroom extract(s) comprised within said composition, and said (additional) active agent, are altogether referred to herein as “active ingredients”.

In certain embodiments, said active agent is a cannabinoid, a combination thereof, or a Cannabis plant extract comprising said cannabinoid or combination thereof.

The term “cannabinoid” as used herein refers to a chemical compound acting on cannabinoid receptors, i.e., a cannabinoid type 1 (CB1)- or cannabinoid type 2 (CB2)-receptor agonist. Ligands for these receptor proteins include the endocannabinoids produced naturally in the body; the phytocannabinoids found in Cannabis sativa and some other plants; and synthetic cannabinoids. The cannabinoid that may be comprised within the composition of the invention may be derived from a Cannabis extract, e.g., Cannabis Sativa extract, using any suitable extraction and purification procedures known in the art. Alternatively, said cannabinoid may be synthesized following any one of the procedures disclosed in the literature.

In certain embodiments, the cannabinoid comprised within the composition of the invention is selected from cannabidiol (CBD), cannabidiolic acid (CBDA), cannabidivarin (CBDV), cannabidivarinic acid (CBDVA), cannabidiol monomethyl ether (CBDM), cannabidiol-C4 (CBD-C4), cannabidiorcol (CBD-C1), Δ⁹-tetrahydrocannabinol (Δ⁹-THC), Δ⁹-tetrahydrocannabinolic acid (Δ⁹-THCA), Δ⁹-tetrahydrocannabivarin (Δ⁹-THCV), Δ⁹-THCVA, Δ⁸-THC, Δ⁸-THCA, Δ⁸-THCV, Δ⁸-THCVA, iso-tetrahydrocannabinol-type (iso-THC), cannabinol (CBN), cannabinolic acid (CBNA), cannabinol-C4 (CBN-C4), cannabinol-C2 (CBN-C2), cannabiorcol (CBN-C1), cannabinol methyl ether (CBNM), cannabinodiol (CBND), cannabigerol (CBG), cannabigerovarin (CBGV), cannabigerolic acid (CBGA), cannabigerovarinic acid (CBGVA), cannabigerol monomethyl ether (CBGM), cannabigerolic acid monomethyl ether (CBGAM), cannabichromene (CBC), cannabichromenic acid (CBCA), cannabichromevarin (CBCV), cannabichromevarinic acid (CBCVA), cannabichromanon (CBCN), cannabicyclol (CBL), cannabicyclolic acid (CBLA), cannabicyclovarin (CBLV), cannabivarin (CBV), cannabivarinic acid (CBVA), cannabielsoin (CBE), cannabielsoic acid A (CBEA-A), cannabielsoic acid B (CBEA-B), cannabitriol (CBT), cannabitriolvarin (CBTV), ethoxy-cannabitiolvarin (CBTVE), cannabifuran (CBF), dehydrocannabifuran (DCBF), cannabiripsol (CBR), an enantiomer, diastereomer, or pharmaceutically acceptable salt thereof, or a mixture thereof.

In particular embodiments, the cannabinoid comprised within the composition of the invention is CBD (2-[(1R,6R)-6-isopropenyl-3-methylcyclohex-2-en-1-yl]-5-pentylbenzene-1,3-diol), or an enantiomer, diastercomer, or a mixture, e.g., racemate, thereof, preferably CBD; or a Cannabis plant extract, e.g., Cannabis Sativa extract, comprising CBD, or said enantiomer, diastereomer, or mixture thereof.

CBD has two stereogenic centers, i.e., at positions 3 and 4 of the cyclohexenyl ring, and may accordingly exist as an enantiomer, i.e., an optical isomer (R or S, which may have an optical purity of 90%, 95%, 99% or more), racemate, i.e., an optically inactive mixture having equal amounts of R and S enantiomers, a diastereoisomer, or a mixture thereof. The present invention encompasses compositions wherein the active agent is any one of such enantiomers, isomers and mixtures thereof.

CBD may be synthesized following any one of the procedures known in the art, e.g., by acid condensation of p-mentha-2,8-dien-1-ol with olivetol. Optically active forms of CBD may be prepared using any one of the methods disclosed in the art, e.g., by resolution of the racemic form by recrystallization techniques; chiral synthesis; extraction with chiral solvents; or chromatographic separation using a chiral stationary phase. A non-limiting example of a method for obtaining optically active materials is transport across chiral membranes, i.e., a technique whereby a racemate is placed in contact with a thin membrane barrier, the concentration or pressure differential causes preferential transport across the membrane barrier, and separation occurs as a result of the non-racemic chiral nature of the membrane that allows only one enantiomer of the racemate to pass through. Chiral chromatography, including simulated moving bed chromatography, can also be used. A wide variety of chiral stationary phases are commercially available.

In particular embodiments, the composition of the invention comprises a Cannabis plant extract comprising a cannabinoid as referred to hereinabove, e.g., CBD. Such an extract may be obtained utilizing any method or technique known in the art, or may alternatively be a commercially available product. In specific such embodiments, the Cannabis plant extract is obtainable from the seeds of said Cannabis plant such as hemp oil (hempseed oil).

In certain embodiments, the composition disclosed herein comprises an extract as defined in any one of the embodiments above, e.g., an extract obtained by extracting said higher Basidiomycetes mushroom or combination thereof with water, or a combination of said extract and an additional extract obtained by extracting said higher Basidiomycetes mushroom or combination thereof with an alcohol, and CBD or enantiomer, diastereomer, or mixture thereof, preferably CBD. In particular such embodiments, said composition comprises said CBD or enantiomer, diastercomer, or mixture thereof in an amount of, e.g., up to 1000 mg, i.e., up to 0.5, 1, 5, 10, 20, 30, 40, 50, 100, 250, 500, or 750 mg. In other particular such embodiments, the amount of said CBD or enantiomer, diastercomer, or mixture thereof constitutes, e.g., up to 0.5%, 1%, 2%, 5%, 10%, 15%, 20%, 25%, or 50%, by weight, of the overall amount of active ingredients in said composition.

The compositions disclosed herein according to any one of the embodiments above may be nutraceutical compositions optionally further comprising a nutraceutically acceptable carrier; or a cosmetic composition optionally further comprising a cosmetically acceptable carrier.

In certain embodiments, the composition of the invention is a nutraceutical composition. In particular such embodiments, said nutraceutical composition is a liquid, semi-solid, or solid, formulated as a food/dietary supplement in the form of, e.g., tablets, caplets, pills, hard or soft capsules, troches, lozenges, or dispersible powder or granules. In other embodiments, said nutraceutical composition is comprised within a drink or beverage.

The nutraceutical composition disclosed herein is a “Dietary Supplement” as established by the F.D.A. in the Dietary Supplement Act of 1994, according to which a dietary supplement includes vitamins, minerals, herbs or other botanicals, antioxidants, amino acids, or other dietary substances used to supplement the diet by increasing the total daily intake. As with pharmaceutical compositions, the amount of active ingredients in the nutraceutical composition will depend on several factors, but will generally be sufficient to provide a consumer with an effective amount of the active ingredients upon consumption of a regular (e.g., daily) portion of the composition.

In certain embodiments, the composition of the invention is a cosmetic (cosmeceutical) composition for topical application to the skin or to a mucous membrane of a subject. Such cosmetic compositions may be formulated, e.g., as a solution, emulsion, suspension, lotion, gel, cream, paste, powder, soap, surfactant-containing cleanser, oil, powder foundation, emulsion foundation, wax foundation, and spray. For example, such a composition may be prepared in a dosage form of an emollient lotion, nourishing lotion, nourishing cream, massage cream, essence, eye cream, cleansing cream, cleansing foam, cleansing water, pack, spray, and/or powder. The cosmetic compositions may include, as carriers, ingredients ordinarily used in cosmetic compositions, e.g., ordinary supplements such as antioxidants, purifiers, solubilizers, vitamins, pigments and/or flavoring agents. As with pharmaceutical compositions, the amount of active ingredients in the cosmetic composition will depend on several factors, but will generally comprise a concentration that is sufficient to provide a consumer with an effective amount of the active ingredients combination upon consumption of a regular (e.g., daily) portion of the composition.

In yet a further aspect, the present invention relates to a method of providing a dietary supplement rich in amino acids, phenolic compounds, flavonoid compounds, and/or antioxidant substances to a subject, said method comprising administering to said subject a nutraceutical effective amount of a nutraceutical composition as defined above.

The term “subject” as used herein refers to any mammal, e.g., a human, non-human primate, horse, ferret, dog, cat, cow, and goat. In a preferred embodiment, the term “subject” denotes a human, i.e., an individual.

In still a further aspect, the present invention relates to use of a nutraceutical composition as defined above as a dietary supplement rich in amino acids, phenolic compounds, flavonoid compounds, and/or antioxidant substances.

Nutraceutical compositions as disclosed herein are rich in various active ingredients originating from the higher Basidiomycetes mushrooms extracted, e.g., amino acids, phenolic compounds, flavonoid compounds, and/or antioxidant substances, and may thus be useful in treating various medical diseases, disorders or conditions in which provision of such active ingredients may be beneficial. As shown herein, for example, a particular composition comprising a combination of the specific extract identified herein as Extract 15 and an extract obtained by extracting Ganoderma lucidum, e.g., Ganoderma lucidum CBS 146996, with ethanol (e.g., 70%), at a ratio of about 9:1, respectively, by volume, was found to have a broad anti-inflammatory activity and may thus be useful in treatment of chronic inflammatory diseases.

Unless otherwise indicated, all numbers expressing, e.g., amounts of components and temperature, used in this specification, are to be understood as being modified in all instances by the term “about”. Accordingly, unless indicated to the contrary, the numerical parameters set forth in this specification are approximations that may vary by up to plus or minus 10% depending upon the desired properties to be obtained by the present invention. The invention will now be illustrated by the following non-limiting Examples.

### EXAMPLES

### Example 1. Deposition of Medicinal Mushroom Strains

The following seven mushroom strains, out of the sixteen referred to herein, were deposited in the Centraalbureau voor Schimmelcultures CBS-KNAW culture collection:
CBS 146994 Coprinus comatus (O.F. Müll.) Pers.
CBS 146995 Flammulina velutipes (Curtis) Singer
CBS 146996 Ganoderma lucidum (Curtis: Fr.) P. Karst.
CBS 146997 Grifola frondosa (Dicks.) Gray
CBS 146998 Hericium erinaceus (Bull.: Fr.) Pers.
CBS 146999 Pleurotus ostreatus (Jacq.: Fr.) Kummer
CBS 147000 Trametes versicolor (L.: Fr.) Lloyd

### Example 2. Description and Characteristics of the Medicinal Mushroom Species Used Agaricus brasiliensis Wasser et al

Description A. brasiliensis is mushroom rich in bioactive compounds such as organic acids, amino acids, phenolic compounds, and polysaccharides such as β-glucans. Moreover, it comprises compounds such as lactic and fumaric acid, as well as secondary metabolites such as sesquiterpenes, steroids, anthraquinones, quinolines and derivatives of benzoic acid, inhibitors of bacterial growth. In addition, A. brasiliensis has a high antioxidant potential mainly due to the presence of phenolic compounds such as gallic acid, serum acid and pyrogallol, karmic acid and other compounds such as ascorbic acid and α-tocopherol. Numerous health promoting effects of A. brasiliensis have been scientifically proved. Immunomodulatory properties, antigenotoxic, antioxidant, anticarcinogenic, antiherpetic, antimutagenic and antitumor activities have been described. All these beneficial effects were demonstrated on mammals or mammalian cells in vitro and with the aim of promoting human health. Moreover, it is often consumed as food and tea worldwide, due to its medicinal effects such as relief of physical and emotional stress, treatment for high cholesterol, diabetes, gastric disorders, and osteoporosis, and improvement of liver function in patients with hepatitis B (Hsu et al., 2008; Li et al., 2020).

Characteristics. Vegetative mycelium in pure culture: Malt Extract Agar (MEA); Potato Dextrose Agar (PDA), optimal growth temperature 27° C. on MEA or PDA, appropriate growth pH 6.0. Teleomorph: The cap is initially hemispherical, later becoming convex, with a diameter of 5-18 cm. The cap surface is covered with silk-like fibers, although in maturity it develops small scales (squamulose). The color of the cap may range from white to grayish or dull reddish-brown; the cap margin typically splits with age. The gills are not attached to the stalk (free), narrow, and crowded closely together. They start out whitish in color, then later pinkish and finally black-brown as the spores mature. Spores are ellipsoid, smooth, dark purplish-brown when viewed microscopically, with dimensions of 6-7.5 by 4-5 μm. The stipe is 6-15 cm (2.4-5.9 in) by 1-1.5 cm (0.4-0.6 in) thick, and bulbous at the base (Wasser et al. 2002, 2005, 2011).

### Auricularia Aricula-Judae (Bull.) J. Schröt.

Description A. aricula-judae is known as medicinal/edible mushroom. The most abundant functional components in this species of mushrooms are polysaccharides and phenolic compounds. Polysaccharides mainly are presented as a D-glucose residue backbone with various chains of β-1,3-branch residues such as mannose, glucose, xylose, and glucuronic acid.

Due to its high nutritional value, A. auricula-judae is widely used in traditional Asian medicine. Except polysaccharides and phenolic substances, A. auricula-judae is significantly rich in flavonoids, alkaloids, saponins, tannin, organic acids, ergosterol, proteins, fats, pigments, vitamins, and micro-macro elements (Poniedziałek et al., 2019). It has been proven that bioactive substances (mainly polysaccharides) from Auricularia species not only exhibit an extremely high nutritional value but also has various pharmacological functions and benefits in humans. Many biological functions of bioactive compounds isolated from A. auricula-judae have been reported by several investigations, such as antimicrobial and antioxidant activity, immunomodulatory activity, hypoglycemic or anti-diabetic properties, radio-protective properties, anti-inflammatory and anti-tumor properties, or anti-viral properties, anti-cardiovascular and anti-hypercholesterolemic properties (Poniedziałek et al., 2019). The beneficial effect of lowering elevated serum cholesterol level in preventing coronary heart disease is well established. It was shown by Jeong et al. (2007) cholesterol lowering, and hypolipidemic effects of biopolymers extracted from mycelial biomass of A. aricula-judae.

Characteristics of Auricularia auricula-judae (Bull.: Fr.) Quel. Vegetative mycelium in pure culture: MEA; PDA, optimal growth temperature 25-28° C. on MEA or PDA, appropriate growth pH 6.0. Mycelial colony white, cottony when fresh. Pigment secretion was observed during mycelial growth on PDA. The average growth is 5.6 mm/day. Teleomorph: Basidiocarps gelatinous when fresh, yellow to dark brown, superior surface pilose, inferior surface with few folds, 3.5-6 cm wide; substipitate, 1.3-1.7 cm long, cylindrical and solid; pilose zone shows hairs of 30-150 μm long, hyaline, often with broken tips. Compact zone 20-90 μm in width. Superior subcompact zone 20-100 μm wide. Intermedia laxa 120-500 μm wide. Inferior subcompact zone 40-95 μm wide. Hymenium 60-80 μm wide; basidia 40-70 μm. Spores allantoids 16-19 μm long and 4.3-4.7 wide (Wasser and Weis, 1999; Jo et al., 2014).

Coprinus comatus (O.F. Müll.) Pers.

Description Many reports have indicated the fruiting body and the mycelial biomass of C. comatus, are rich source of polysaccharides, fatty acids, tocopherols, organic acids and phenolic compounds, possesses anti-obesity, immunomodulation, hypolipidemic. anticancer, hypoglycemic, antioxidant, antibacterial, antidiabetic and hepatoprotective activities (Nowakowski et al., 2021). C. comatus generates 4,5-dihydroxy-2-methyoxy-benzaldehyde, also known as comatin, a compound with a hypoglycemic effect (Ding et al. 2010). Experimental data collected in the study provided by Akata et al. (2019) support the use of C. comatus as functional food for the management and/or prevention of diabetes type II, Alzheimer's disease, and oxidative stress related complications. It was reported that extracts from submerged mycelial biomass of C. comatus were potent and promising in the production of certain phenolic compounds and can be recommended for further biotechnological processes in the production of highly valuable natural antioxidants, which have wide prospects for use in cosmetics and pharmaceuticals.

Characteristics [CBS 146994]. Vegetative mycelium in pure culture: MEA; PDA, optimal growth temperature 27° ° C. on MEA or PDA, appropriate growth pH 6.0. Mycelial colony white, cottony, often develops “tufts” (hyphal aggregates) with maturity. Asymmetrically shaped mycelial mat often forms along the outlet edge of colony. Clamp connections, anastomoses, and hair-like crystals are often present on hyphac. Telcomorph: Pileus 3-15 cm; oval to rounded-cylindrical when young, expanding to bell-shaped with a lifting margin; in age turning to black “ink”; dry; whitish with a brownish center; with large, shaggy scales; margin lined at maturity. Lamellae free from the stem; white, becoming pinkish, then black; turning to black “ink”; very crowded. Stipe 5-20 cm long; 1-2 cm thick; frequently tapering to apex; smooth; white; casily separable from cap; hollow, with a string-like strand of fibers hanging inside. Context white throughout, soft. Spores print black. Basidiospores 9-13×7-9 μm; elliptical; smooth; with a central to slightly eccentric pore. Basidia 4-spored, 28-43×10-13 μm, surrounded by 5-8 pseudoparaphyses. Pleurocystidia absent. Cheilocystidia variously shaped; up to 60×40 μm. Pilcipellis made of cylindric elements 7-30 μwide. Only pseudoclamps presents (Moser, 1983, Wasser and Weis, 1999, Kosakyan et al., 2014, Wasser 2011).

Cordyceps militaris (L.) Link

Description Various extracts from C. militaris were reported to have different pharmacological activities, including antitumor, antihyperglycemic, antiinflammatory, immunomodulatory and antioxidant activities (Lec et al., 2020a). It was demonstrated that cultivate C. militaris can induce apoptosis and autophagy in human glioblastoma cells. In addition, some of the active compounds identified in this fungus have been tested in preclinical trials and clinical trials (He et al., 2020). It was established that besides extracts obtained using organic solvents, water extracts were also potent to have various pharmaceutical effects (Lec et al., 2020a). Water extracts obtained from fruiting body of C. militaris and found to have potential therapeutic effects on asthma in an ovalbumin (OVA)-induced asthma mice model (Hsu et al., 2008). Han et al. (2011) found that the water extract of mycelium of C. militaris attenuated dextran sodium sulfate-induced acute colitis in mice. Polysaccharides are crucial bioactive components in C. militaris. It was found that the immunomodulatory effects of C. militaris polysaccharides might be associated with binding TLR2, TLR4, and dectin-1 on immune cells. Notably, polysaccharides derived from C. militaris, was demonstrated to have perform two types of functions, namely immune stimulation and anti-inflammation. Most crucial and valuable bioactive substance derived from C. militaris is cordycepin discovered in the early 1950s. Wild type C. militaris possesses a low content of cordycepin and has restricted growth in natural mycelial biomass. To overcome these limitations, several studies successfully attempted to enhance cordycepin production in its mycelial biomass in vitro under submerged conditions by adding various growth supplements. Cordycepin (30-deoxyadenosine) is the signature bioactive compound of Cordyceps spp. which is considered as nucleoside antibiotic and possesses immense pharmacological activities such as antimicrobial, antiviral, antioxidant, antifungal, anti-angiogenesis, immunomodulatory, anti-pigmentation and antitumor. Beside several therapeutic effects of cordycepin, its human random clinical trials depicted a promising anti-inflammatory activity that reduced the airway inflammation remarkably in asthmatic patients. Moreover, cordyceps successfully can be used as a stimulant, a tonic, and an “adaptogen,” which is used to increase energy, improving athletic performance, reducing the effects of aging, reduce fatigue and enhance stamina.

Characteristics of Cordyceps militaris (L.: Fr.) Fr. Vegetative mycelium in pure culture: MEA; PDA, optimal growth temperature 24° C. on MEA or PDA, appropriate growth pH 6.0. Beside this, vegetative mycelium of C. militaris can successfully grow in a wide range of media, suggesting that it is a facultative phagotrophic mushroom in nutrition habit. Entomopathogenic fungi have been found to survive well with restricted growth in soil for a long period of time without infecting its host. The average mycelium diameter of C. militaris range from 4.90 cm to 5.78 cm and mycelium density is compact at 12-14 days after inoculation. Telcomorph: The host of C. militaris (entomopathogenic fungi) is Lepidopteran pupa. 15-30×2-3 mm, clavate, indistinctly separated into head and stalk. Head fertile, irregularly clavate to cylindrical, orange-yellow when fresh, rouge and punctuate with projecting peritecia. Stalk sterile, pale orange to ochre, sometimes mottled with orange, smooth distinctly set off from the head. Arising slightly to clustered from stromatized pupal tissue. Hyphae with simple septa, 3-3.8 μm in diam. Asci eight-spored, breaking apart into many individual ascospores up to 250×5 μm. Ascospores thin cylindric, hyaline, thin-walled, smooth, 5-6×1.5-2 μm, lined up on each other, inside the ascus like a chain (Wasser, 2011).

Flammulina velutipes (Curtis) Singer

Description F. velutipes is one of the most common economic edible/medicinal mushroo, which is recognized for its high organoleptic properties and good nutritional value. Several investigations suggested that F. velutipes and bioactive substances thereof successfully can be used as a precious source in pharmacology and the health care industry. Several biological and pharmacological functions of this edible/medicinal mushroom have been identified. It was indicated that F. velutipes polysaccharides has antitumor, memory and learning improvement, antioxidant activity, immunomodulatory properties, and hepatoprotective activity (Wang et al., 2018a). In addition, F. velutipes exopolysaccharides have considerable antioxidant capacity based on reducing hydroxyl radical, superoxide anion radical, reducing power and ABTS/DPPH scavenging properties. From the aqueous extract of fruit bodies of F. velutipes, flammulin, an anti-tumor substance, was purified. A stable hemagglutinin was isolated from the fruiting bodies of this mushroom, which inhibits proliferation of leukemia L1210 cells. F. velutipes has low calorie and fat contents but is rich in fiber, carbohydrates, proteins, and the essential fatty acids 18:2n-6 and 18:3n-3. Moreover, F. velutipes also contains bioactive substances with potential influence against hypercholesterolemia, atherosclerosis, thrombosis, hypertension as well as antiaging, antiallergic, antimicrobial, and hepatoprotective properties. It was reported by Wu et al. (2014), that water-soluble polysaccharides (with a molecular mass of 8.14×10 Da) obtained from F. velutipes could scavenge hydroxyl radical, superoxide anion and possessed reducing power and possessed antioxidant activity and could enhance non-specific and specific immune responses in vitro. Beside pharmacological effects, it was determined that, among twenty-nine screened fungi, F. velutipes was regarded as an ideal species to potentially drive a sensorial tea drink because of its appealing nutty and chocolate-like flavor and a rapid fermentation process, during 16 h (Rigling et al., 2021).

Characteristics [CBS 146995]. Vegetative mycelium in pure culture: MEA; PDA, optimal growth temperature 27° C. on MEA or PDA, appropriate growth pH 6.0. Mycelial colony white, aerial mycelium longitudinally linear, becoming finely appressed and tinged light brown to spotted with golden yellow-brown zones with age. The surface roughens at the earliest stage of primordia formation. Long-stemmed, small-capped mushrooms commonly form along the inside periphery of the Petri dish on agar media. Conidial sporulation's (arthroconidia) are presented on hyphae. Teleomorph: Pileus (1)2-6(10) cm in diam., convex, then expanded and plane and sometimes irregularly undulating, surface smooth, lubricous, viscid, yellow to orange-brown, darker toward the center, margin paler, acute and even or faintly striate, cuticle gelatinous, partly peelable. Lamellae whitish when young, then pale orange yellow, broad, adnate to slightly notched, sometimes anastomosing, edges smooth and concolorous. Stipe 1.5-7(10)×0.2-1(1.5), central, cylindric, at times somewhat widened at the apex, brownish to black, apex yellowish, entierly velutinous, sometimes strigose toward the base, fibrous, solid, soon hollow. Context mild, soft, cream colord, odor pleasant, taste mild, nutty. Spores print whitish. Basidiospores cylindric-elliptic, 8-11×3.2-4.5 μm. Basidia 4-sterigmata and basal clump, slenderly clavate, 35-40×4-5 μm. Cheilocystidia slendery clavate, 45-65×9-13 μm, numerous, dermatocystidia cylindric-clavate, somewhat thick-walled, with yellowish contents, brown in KOH, 60-120×6-10 μm. Caulocystidia fusiform, brownish, up to 300×20 μm. Pileipellis made of irregular, abundantly and multiply branched hyphae 2-3 μm across, septa with clamps, gelatinized, with dermatocystidia (Moser, 1983, Wasser 2011, Kosakyan et al., 2014).

Ganoderma lucidum (Curtis: Fr.) P. Karst.

Description It has been reported that G. lucidum (also called the “mushroom of immortality”) yields miraculous health benefits and can produce remarkable wide variety (over 400) bioactive compounds, including triterpenoids, different carbohydrates including glycoproteins and polysaccharides, nucleotides and their derivatives, minerals, phenolic compounds and flavonoids, sterols, steroids, different volatile compounds, fatty acids (polyunsaturated fatty acids between them) and proteins/peptides, which are substantial contributors to the high health importance, and therefore have a number of medicinal effects (Sohretoglu and Huang, 2018; Lcc et al., 2020b, Li et al., 2020). Nowadays G. lucidim is one of the most extensively studied medicinal mushroom. Modern medicinal studies have demonstrated that this mushroom possesses a broad range of bioactivities, including anti-inflammatory, antioxidant, anti-glycemic, antiulcer, anti-cancer, and immune-stimulating, cholesterol lowering, antidiabetic, liver protective effects (Sohretoglu and Huang, 2018, Xie et al., 2019, Lee et al., 2020b). Hence, G. lucidum has been used to treat a variety of chronic diseases. Moreover, G. lucidum, has also reported to treat Alzheimer's disease effectively (Wang et al., 2018b).

Nowadays, several types of mushrooms including G. lucidum are incorporated in topical creams, lotions, ointments, and facial preparations as cosmetic ingredients such as phenolics/polyphenolics, terpenoids, selenium, polysaccharides, vitamins, and volatile organic compounds. These compounds show excellent antioxidant, anti-aging, anti-wrinkle, skin whitening, and moisturizing effects, which make them ideal candidates for cosmetics products (Wu et al., 2016).

Beside this, it was reported that fatty acid rich Gano oil extracted from G. lucidum is an antinociceptive agent capable to inhibit oedema by oral administration. The finding suggests that Gano oil might be a potent natural product-based analgesic. The effect might be assigned to the fatty acid amide constituents especially oleamide which has been demonstrated to have analgesic action. In addition, G. lucidum mushroom seems to be able to improve fibromyalgia symptoms, including depression and pain (Pazzi et al., 2020). Moreover, G. lucidum is listed in the American Herbal Pharmacopoeia and Chinese Pharmacopoeia, and was attributed with therapeutic properties such as tonifying effects, enhancing vital energy, strengthening cardiac function, increasing memory, and antiaging effects.

Characteristics [CBS 146996]. Vegetative mycelium in pure culture: MEA; PDA, optimal growth temperature 27° C. on MEA or PDA, appropriate growth pH 6.0. Mycelial colony white, dense with concentric zones, cottony, felty in the centre, subfelty with pronounced hyphal tufts near the edge of the Petri dish, interwoven, mycelium at the margin is raised. Reverse unchanged. Vegetative mycelium in colony centre has several types of hyphae. Clamp connections and aggregations of exometabolites are presented on hyphac. Telcomorph: Pileus 2-20 cm; at first irregularly knobby or clongated, but by maturity more or less fan-shaped; with a shiny, varnished surface often roughly arranged into lumpy “zones”, red to reddish brown when mature, when young often with zones of bright yellow and white toward the margin. Pore surface white, becoming dingy brownish in age; usually bruising brown; 4-7 tiny (nearly invisible to the naked eye) circular pores per mm, tubes to 2 cm deep. Stipe sometimes absent, but more commonly present; 3-14 cm long; up to 3 cm thick; twisted; equal or irregular, varnished and colored like the cap, often distinctively angled away from one side of the cap. Context brownish, fairly soft when young, but soon tough. Spore print brown. Basidiospores 7-13×5-9 μm, more or less elliptical, sometimes with a truncated end; with the hyaline germination pore, appearing smooth at lower magnifications, finely spiny at high magnification. Basidia with 4 sterigmata, ventricose, 17-20×10-12 μm. Pileocystidia in regular palisade, clavate, brown, thick walled, with superficial resinous excretion, amyloid, 60-125×7.0-13 μm. Hyphal system trimitic, generative hyphae sparse, hyaline, 2.0-3.0 μm across, septa with clamps; skeletal hyphae thick-walled, brownish 2.0-7.0 μm across, cyanophilic; binding hyphae thick walled to solid, brownish, 2-3 μm across, strongly branched (Tura et al., 2011, Wasser, 2011, Ryvarden and Melo, 2014, Papp et al., 2017).

Ganoderma tsugae Murrill

Description G. tsugue has been commercially cultivated as a dietary supplement. The chemical composition assay includes amounts of total carbohydrates and proteins, amino acids, fatty acids, micro- and macro-elements, and vitamins. The investigated medicinal mushroom seemed to be a rich source of nutritional components. Mycelium accumulates more than 2-fold more total protein content compared with the fruiting body and reach 37% and 16% of dry weight, respectively (Chan et al., 2015). Both mushroom species (G. tsugae and G. lucidum) are very similar in content of bioactive compounds and biological activities. G. tsugae has found to be active in several therapeutic effects, including antibacteria, blood pressure regulation, immunomodulation, kidney toning, liver protection, sexual potentiating, etc. The most metabolite product recognized in G. tsugae are polysaccharide complexes which are mainly responsible to immunomodulatory and anticancer activities.

Characteristics. Vegetative mycelium in pure culture: MEA; PDA, optimal growth temperature 27° C. on MEA or PDA, appropriate growth pH 6.0. G. tsugue mushrooms are most similar species to Ganoderma lucidum. Mycelial colony white, cottony. Telcomorph: the upper surface is a dark reddish-brown or brownish-orange and so shiny that it looks varnished. Shaped like a giant, furrowed kidney bean or fan, the cap can grow from 5-30 cm wide. Usually, one stalked fruiting body is found growing from a single attachment to the host tree, however sometimes two can grow from one base. The undersurface is tan to white in color and has the appearance of suede leather. A hand lens reveals the fine pores. Pore surface also is whitish, becoming brown in age or when bruised; pores 4-6 per mm. The stalk when present is usually attached laterally and is 2.5-15 cm long, 1-4 cm thick and also reddish brown and varnished (Adaskaveg and Gilbertson, 1986, Wasser et al., 2011).

Grifola frondosa (Dicks.) Gray

Description G. frondosa considered among the most important medicinal mushrooms in traditional Chinese medicine and Japanese medicine. Different water-soluble and non-water-soluble biologically active substances can be obtained from G. frondosa by using different extraction methods, hot water extraction between them. G. frondosa has received an enormous amount of attention as a dietary supplement due to its high nutritional value. The active ingredients mainly are glycoproteins and polysaccharides. A large number of pharmacological analyses and clinical experiments confirmed that polysaccharides obtained from G. frondosa have significant antitumor and antiviral activities (hepatitis B, human immunodeficiency virus), and can improve the immune system, regulate blood sugar, blood fat and cholesterol levels, blood pressure, and exert other effects, without any side effects. To demonstrate and confirm such biological activities, numerous studies have been performed in vitro and in vivo or in clinical settings. Most of fungi polysaccharide preparations are invalid when taken orally. In addition, injection is also effective and convenient for clinical application and studies (He et al., 2018). Extracts obtained from G. frondosa is one of the components (with Agaricus brasiliensis and Hericium erinaceus) of Andosan™ (anti-allergic and anti-inflammatory ingredient), which has been extensively investigated in clinical studies and established its immunomodulating properties. Moreover, this combination of medicinal mushroom species demonstrates antimicrobial activity against viral agents, Gram-positive and Gram-negative bacteria, and parasites in vitro and in vivo. It is possible to view G. frondosa as an antiseptic means. Since the mechanism is immunomodulatory and not antibiotic, the mushrooms should be active against multi-drug resistant microbes as well. Moreover, since these Basidiomycota also have anti-inflammatory properties, they may be suited for treatment of the severe lung inflammation that often follows COVID-19 infection (Hetland et al., 2020). β-glucan extracted from G. frondosa has been reported to enhance antitumor activity and bone marrow toxicity by strengthening the immune system (Masuda et al., 2009). Furthermore, it is reported that G. frondosa polysaccharide increased the metabolism of glucose and stimulated synthesis of intracellular glycogen through the Akt/GSK-3 pathway (Ma et al., 2014). It was shown antitumor and antimetastatic activity of G. frondosa extracts agains breast cancer cells. Potential therapeutic role of standardized form of protein-bound β-glucans (proteoglucan) in aggressive triple-negative breast cancer (TNBC) was established (Alonso et al., 2018). Standardized beta-glucan extracts such as the D- and MD-fractions are recognized as carcinostatic agents that can be used in conjunction with conventional medical treatments to treat cancer. In conclusion, G. frondosa is among the most promising natural sources of immunotherapeutic products.

Characteristics [CBS 146997]. Vegetative mycelium in pure culture: MEA; PDA, optimal growth temperature 27° C. on MEA or PDA, appropriate growth pH 6.0. Mycelial colony white, later light tawny-brown tones longitudinally linear, eventually thickly, cottony, non-rhizomorphic. The mycelium grows out eventually. At mature, the dense mycelial mat can be peeled directly off the agar media. Mycelia usually developed strong yellowish to orange-brown mottled zones, with drops of yellowish metabolite. In the vegetative mycelium numerous medallion clamp connections are present, anastomoses between hyphae and clamps occurred. In the younger part of mycelial colony branched thin (1 μm) hyphae (dichohyphidia) are formed. Apical and intercalary chlamydospores form on hyphae, which usually have no clamp connections. Teleomorph: Pileus cluster 15-60 cm broad or more; individual caps 2-10 cm, fan-shaped, gray-brown (often with concentric zones), with wavy margins. Pore surface lavender gray when young, becoming dirty whitish to yellowish, running down the stem(s). Stipe branches smooth, white, tough, usually lateral. Context firm, white, tough, taste mild. Spore print white. Basidiospores 5-7×3.5-5 μm; smooth; broadly elliptical. Basidia 4-sterigmata, clavate, 20-25×6-8 μm. Cystidia not obsereved. Hyphal system monomitic, subhymenial hyphae thin-walled, 2-3.5 μm across, sparsely septate, some with clamps; tramal hyphae thick-walled, often bladderliked and short-called, 10-40 μm across (Buchalo et al., 1999, Tura et al., 2011, Sharma, 2012, Ryvarden and Melo, 2014).

Hypsizygus marmoreus (Peck) H. E. Bigelow

Description H. marmoreus is a well-known, commercially cultivated edible mushroom which has a great organoleptic and medicinal properties. Besides the wonderful organoleptic quality, H. marmoreus has been widely considered to be nutritional supplement for health maintenance and disease prevention contributing to its beneficial components.

During the past years, dozens of clinical examinations based on somewhat different strategies have shown that polysaccharides from H. marmoreus expressed potential bioactivities. Studies have shown that the polysaccharide extracted from the fruit body and mycelial biomass of H. marmoreus has a series of physiological activities such as anti-tumor, hypolipidemic, hypoglycemic, immune regulation, antioxidant and anti-inflammatory effects, prevention of aging, and prolonging life (Liu et al., 2018). Several bioactive molecules have been reported to underlie the medicinal effects of H. marmoreus; in particular, the terpenoid compound hypsiziprenol A9 inhibits cell cycle progression in HepG2 cells, a human liver cancer cell line. It was demonstrated by Liu et al. (2018) that mycelial selenium polysaccharides might be a potentially effective candidate medicine for the treatment of lung damage and its complications. Moreover, it was suggested that the extracts obtained from H. marmoreus are effective sources of syringic acid and vanillic acid, which are effective in preventing osteoporosis (Tanaka et al., 2019).

Characteristics. Vegetative mycelium in pure culture: MEA; wort agar (WA), optimal growth temperature 22-26° C. on MEA or PDA, appropriate growth pH 6.0. H. marmoreus strains favored MEA and WA the most, with pH of 5.5 and 6.0, respectively. It has white mycelia of plumule shape, no thick acrial hypha, no yellow liquid exudation and no coat. It may produce arthrospore and chlamydospore during the culture. Hypsizygus marmoreus has a relative stronger resistance to bad environment. Changing temperature is needed in the fruiting of this mushroom. The premordium differentiation require 4-18° C., and the optimum is 10-14° C. Teleomorph: The morphological changes under standard cultivation conditions observed after mycelial maturation following the decrease in temperature and increase in light intensity (50-200 lux). Under these conditions, the mycelium turns into fluffy hyphal knots that were 0.5-1.0 mm in diameter and white in color. The mycelium transforms from vegetative growth to reproductive growth, and the color changes to gray/brown in 3-4 days. After the mycelial pigmentation stage, the pinning fruit body appears in 3-5 days at the primordium stage. When the light intensity will be increased to 200-1000 lux after 6-8 days, the fruit body matured, and the fruit bodies will be composed of long stipes and closed caps (light brown or white umbrella-shaped small cap). Color turns to gray/brown to creamy brown when mature. which will have spotted with water-spots and had a creamy brown color (Buchalo et al., 2011, Zhang et al., 2015).

Hericium erinaceus (Bull.: Fr.) Pers.

Description Hericium erinaceus is one of the well-studied edible/medicinal mushrooms which is a great source of structurally diverse compounds, and about seventy different secondary metabolites. The chemical composition and bioactive substances of fruit bodies and mycelia of H. erinaceus have been reported by several researchers.

Wide range of different biologically active compounds such as hericerins (aromatic compounds such as hericerin, isohericenone, isoericerin, hericerin, N-dephenylethyl isohericerin, hericenone, resorcinols, crinacerins, and hericenols), crinacines (crinacine A, and diterpenoids), crinacerins-isoindolin-1-ones (erinacerins C-L), crinaccolactones, glycoprotein (polysaccharideprotein), polysaccharides (β-D-glucans), sterols (ergosterol, and crinarols), vitamin B12, and volatile compounds (2-methyl-3-furanthiol, 2-ethylpyrazine, and 2,6-diethylpyrazine) have been reported to be present in the H. erinaceus (Chong et al., 2020). Because of the ability to accumulate these and many other biologicaly active compounds, H. erinaceus is one of the most famous medicinal mushrooms with health promoting ability. It is known that the dried powder of H. erinaceus fruiting body and submerged mycelial biomass is rich in proteins, carbohydrates, and amino acids (therefore has a high energy value), whereas contain low fat content. Some potential bioactive compounds such as γ-aminobutyric acid (GABA), ergothioneine, and lovastatin were also found to be reported in H. erinaceus (Cohen et al., 2014). Bioactive compounds extracted from its fruiting body or mycelium have been demonstrated to possess antioxidative, antidiabetic, anticancer, anti-inflammatory, antimicrobial, antihyperglycemic, anti-fatigue, immunostimulatory and hypolipidemic properties (Chong et al., 2020). Moreover, H. erinaceus has been used to treat cognitive impairments, Alzheimer's disease, Parkinson's disease, ischemic stroke, and presbycusis (Chong et al., 2020). H. erinaceus polysaccharide has been reported to exhibit the highest 1,1-diphenyl-2-picrylhydrazyl radical (DPPH) scavenging capacity and strongest inhibitory activity on HeLa cells.

H. erinaceus distinguished as an edible/medicinal mushroom and also a delicacy for dietary supplements, therefore has earned much attention as a potential source of various pharmaceutical properties. Therefore, H. erinaceus and different bioactive substance obtained from it can be successfully used for human well-being due to a large number of health benefits. It should be mentioned that no side effects were recognized.

Characteristics [CBS 146998]. Vegetative mycelium in pure culture: MEA; PDA, optimal growth temperature 27° C. on MEA or PDA, appropriate growth pH 6.0. Mycelial colony whitish, forming triangular zones of collected rhizomorphs, radiating from the dense centre of colony, numerous clamp connections are present. They are usually located near each cell septum. Intercalary and terminal chlamydospores and anastomoses between hyphae are usual. Abundant crystals of cubic or rectangular shape are sometimes present on hyphae. Highly branched hyphal segments are expected. Teleomorph: Fruit body annual, compact to lobed, up to 8-40 cm across; consisting of one, unbranched clump of 1-6 cm, soft spines hanging from a tough, hidden “base” that is attached to the tree; white, or in age discoloring brownish to yellowish. Upper surface with short, irregular, sterile spines. Hymenophoral spines on underside 2-5 cm long, regularly crowded. Context homogenous, soft when fresh, tough upon drying, white, not bruising. Spore print white. Basidiospores 4-5.5×5-6.5 μm; elliptical or nearly round; amyloid; smooth or minutely rough. Gleocystidia present as elongated undulating organs with refractive contents. Hyphae hyaline, often swollen up to 20 μm wide, with clamps (Buchalo et al., 1999, Tura et al., 2011, Sharma, 2012, Ryvarden and Melo, 2014).

Inonotus obliquus (Ach. ex Pers.) Pilát

Description. In many countries such as China, Japan, Korea, Russia, and the Baltic countries, extracts of I. obliquus mushrooms were used due to their beneficial effects on the plasma lipid system and heart function as well as antibacterial, anti-inflammatory, anti-fatigue and anti-cancer activity. Furthermore, the antioxidant activity of I. obliquus may play major role for prevention of free radical-related diseases (atherosclerosis, cancer, diabetes, accelerated aging, and degenerative diseases of the central nervous system). Moreover, Chaga extracts have been shown to inhibit the reproduction of viruses (hepatitis C and human immunodeficiency viruses (Szychowski et al., 2021). It should be mentioned, that since the antiviral properties of I. obliquus have been proven, extracts obtained from this mushroom can be promising candidate for treatment and/or prevention of COVID-19 as well (Shahzad et al., 2020). I. obliquus is a source of various bioactive substances: antioxidants, triterpenoids, ergosterol and its peroxide, sesquiterpenes, benzoic acid derivatives, hispidin analogues, andmelanins. In the literature, there are descriptions of research on various substances isolated from I. obliquus, including melanin, inodothiol, flavans, triterpenoids, steroids, polyphenols/flavonoids, or polysaccharide complexes (Szychowski et al., 2021). Many reports have highlighted that the polysaccharide fraction present in I. obliquus extracts is the largest group of active compounds, besides phenols. It was indicated polysaccharide from I. obliquus not only has great potential to postpone physical fatigue but also shown potential to improve mental fatigue. It was described significant the antioxidant potential extracts from I. obliquus, which demonstrated high scavenging activity against ABTS and DPPH free radicals, and moderate activity against the superoxide radical anion. The results obtained by Wold et al., (2020) demonstrate the anti-inflammatory and immunomodulatory effects of I. obliquus melanin and triterpenoids. It was shown that I. obliquus polysaccharides exerts inhibitory effects on lung cancer cells both in vitro and in vivo by decreasing mitochondrial membrane potential, thereby inducing apoptosis of cancer cells. Therefore, I. obliquus polysaccharides can be promising alternative or supplementary medicine for cancer therapy. In conclusion, it can be highlighted that I. obliquus provide a comprehensive landscape view of the pharmacological actions and successfuly can be used for treatment and prevention of different diseases.

Characteristics of Inonotus obliquus (Fr.) Pilat. Vegetative mycelium in pure culture: MEA; PDA, optimal growth temperature 22-24° C. on MEA or PDA, appropriate growth pH 6.0. Teleomorph: I. obliquus (chaga) grows very slowly in nature; it is quite rare and found only in very cold habitats. Chaga produces a woody growth, or conk, which looks similar to a clump of burnt charcoal. Up to 30 cm across and 30 cm high; irregular in outline; erupting through the bark and creating a mass that protrudes 2-5 cm at first but becomes concave over time; surface black, hard, and broken into charcoal-like cubes; dry; exposed flesh orange-brown. However, the inside reveals a soft core with an orange color. I. obliquus produces two stages of fructification: an anamorphic phase and the aforementioned sterile cinder conks: the latter occur perennially and do not produce sexual chlamydospores or a sexual teleomorphic having basidicarps with hymenium, whereas the fertile fruiting body that appears while the host tree or some portion dies forms only once in the infection cycle. Basidiocarps of the teleomorphic stage occur as a layer of pores on cracks respiring on exposed wood, which are processed by special tissues of fungus-infected stem branches. In infected stems, withering occurs after production of basidiocarps. Thus, I. obliquus is connected with living trees like a real parasite. The shape and structure of I. obliquus in the sexual stage is resupinate, forming layers of pores several square decimeters in area and 10˜15 mm in depth. The surface is porous and whitish in the early stage, and then becomes dark brown with a brown gleam in the matured late stage. Pores are angular to oblong. 1˜1.5 mm in diameter, and slant obliquely upward. The basidiospores are hyaline or faintly yellowish, with average dimensions of 8˜12×4˜5 micrometers, having and dark-brown and thick-walled bulbous setae in the hymenium. The anamorphic stage of I. obliquus is more visible and can be observed as a sterile conk. Visually, it is a crispytextured, charcoal-black fungal mass that forms on living stems of birches and alders. The role of chlamydospores produced in sterile conks is not clear. It is presumed that the infection starts from basidiospores (Lim et al., 2005, Lec et al., 2008).

Lentinus edodes (Berk.) Singer

Description L. edodes is a one of the most popular worldwide cultivated edible/medicinal mushroom called shiitake. L. edodes has high nutritional value and is one of the most important sources of β-glucans. Classic extraction procedures of polysaccharides from L. edodes fruiting bodies and submerged mycelial biomass is hot water extraction. It has been confirmed that among different methods examined, hot water extracts enriched in polyphenols and antioxidants to compare to organic-solvent extracts (Xiaokang et al., 2020a).

Several kinds of polysaccharides have been identified in L. edodes with different extraction and purification processes. The β-glucan content in L. edodes is lower than in some other basidiomycetes species, but the water-soluble fraction is much higher. In addition, the water-soluble form has a more pronounced effect on the immune systems of humans, and soluble polar substances present in L. edodes extracts show antioxidant and anticancer properties (Huamán-Leandro et al., 2020). Polysaccharides extracted from L. edodes, named lentinan, caused widespread interest and attention due to non-toxicity and biological activities. Lentinans are a specific class of β-glucans extracted from the edible mushroom L. edodes, and are composed of a ß-(1-3)-glucose backbone with two (1-6)-β-glucose branches of each five glucose units. In addition to polysaccharides, several bioactive compounds including flavonoids, dietary fiber, ergosterol, vitamins B1, B2 and C, and micro-macro elements had been isolated and identified from the fruiting bodies, mycelia, and culture medium of this mushroom. They possess numerous pharmacology properties as reported, such as antimicrobial, antioxidant, anti-tumor, immunoenhancing, liver function improving and hypolipidemic activity (Wang et al., 2019b, Murphy et al., 2020). It was demonstrated significant immunomodulatory and pulmonary cytoprotective effects of polysaccharides (lentinan) which may also have positive relevance to candidate COVID-19 therapeutics targeting cytokine storm (Murphy et al., 2020). There is no doubt that to exploit the application of bioactive compounds, especially polysaccharides, obtained from fruiting bodies and/or mycelial biomass of L. edodes in medicinal and food industries have a vide prospects.

Characteristics. Vegetative mycelium in pure culture: MEA; PDA, optimal growth temperature 27° C. on MEA or PDA, appropriate growth pH 6.0. L. edodes mycelia has a monomictic hyphal system with generative hyphae. Each basidium produces four basidiospores that uninucleate through meiosis. after which the basidiospores germinate to grow into monokaryotic mycelium containing a single haploid nucleus in each cell compartment, with the cell separated by a septum. The dikaryon of L. edodes is typically distinguished by the following morphological characteristics: regular distribution of two haploid nuclei and formation of a clamp connection in cach hyphal cell. The dikaryotic condition usually remains extremely stable during vegetative growth. Teleomorph: The fruiting bodies of L. edodes are generally light-colored to reddish brown or black, with a convex to flat pileus (cap) supported by a fibrous stipe (stalk). The pileus can be 2-25 cm (0.8-10 inches) in diameter, depending on the species, and features white gills on the underside. Lentinula species characteristically produce white spores. The L. edodes fruiting bodies turn brown owing to melanin synthesis (Wasser and Weis, 1999; Song et al., 2018; Ha et al., 2019).

Pleurotus ostreatus (Jacq.: Fr.) Kummer

Description P. ostreatus is a widely cultivated edible mushroom which is famous for its delicious taste and rich contents of carbohydrates, proteins, vitamins, and minerals. The polysaccharides are one of the most studied metabolites of the species P. ostreatus, extracted from fruiting bodies and submerged mycelial biomass. In extracts of P. ostreatus, researchers have found an active β-glucan, named pleuran. The classical way to extract polysaccharides is hot water extraction technique. Various studies have shown that P. ostreatus polysaccharides, which are one of the main bioactive compounds isolated from this mushroom species have offered medicinal activities, including the inhibition of tumor cell proliferation in vivo and in vitro, immune activity regulation, as well as antioxidant, free radical scavenging, and antibacterial effects (Gonzalez et al., 2021). Moreover, it is reported that P. ostreatus polysaccharides are key players in reduction of blood cholesterol concentrations, alleviation of heart disease and blood glucose levels, and it also has been known to prevent liver lipid peroxidation. Beside this, the behavioral test suggested that P. ostreatus polysaccharides were a safe and effective medicine to prevent and treat Alzheimer's disease (Duan et al., 2020).

Chemical composition of P. ostreatus strains are well studied. P. ostreatus has great nutritional value, due to the presence of high contents of amino acids (arginine, alanine, glutamine, glutamic acid), vitamin C, oleic acid, linolenic acid and other compounds with hypocholesterolemic action. One of the most important compounds in P. ostreatus is lovastatin—an approved to market drug used in the treatment of dyslipidemia. Moreover, considerable content of ergothioneine and GABA was reported as well (Cohen et al., 2014). Beside this, the high contents of micro and macro elements such as of potassium, phosphorus, calcium, iron, copper, zinc, magnesium, and selenium were found in fruiting bodies and submerged mycelial biomass of P. ostreatus (Muszyńska et al., 2016). Since P. ostreatus has shown beneficial nutritional properties and wide spectrum of biological activities it can be considered as a promising candidate for production of dietary supplements and pharmaceutical products.

Characteristics [CBS 146999]. Vegetative mycelium in pure culture: MEA; PDA, optimal growth temperature 27° C. on MEA or PDA, appropriate growth pH 6.0. Mycelial colony white, with age cream, greyish to ivory, yellow to orange, dense, cottony, longitudinally radial, with concentric bands of different texture. On agar media telcomorph (primordia and carpophores) are formed. Vegetative mycelium consists of thin-walled and branched hyphae (1.5-7.0 μm). The occurrence of clamp connections on hyphae is typical. Conidial sporulation's of single globose conidia 3-5 μm in diameter are presented on hyphae. Teleomorph: Pileus 4-15 cm, convex, becoming flat or somewhat depressed, kidney-shaped to fan-shaped, or nearly circular if growing on the tops of logs; somewhat greasy when young and fresh; smooth; pale brown to dark brown, the margin inrolled when young, later wavy, never lined. Lamellae decurrent along the upper third of the stipe, close; whitish or with a gray tinge, sometimes yellowish in age; often filled with black beetles, in my collecting areas. Stipe usually rudimentary and lateral (or absent) when the mushroom is growing from the side of a log or tree, when it grows on the tops of logs or branches, or at an angle, however, it may develop a substantial and thick stem that is dry and slightly hairy near the base. Context thick, white, with fungal or sweetish odor, with mild and pleasant taste. Spore print white-pink when fresh, cream, ochraceous, grey when dry. Basidiospores (5.6)-9.5-(13.7)×2.7-3.2-(4.2) μm, cylindric, cylindric-elliptic, amygdaliform, ventral side straight, concave (to convex), dorsal side straight or convex. Basidia with 4 sterigmata and basal clamp, slenderly clavate 25-35×5-7 μm. Cystidia not seen. Hymenophoral trama irregular. Pileipellis irregular, densely intertwined, flexuous and branched hyphae 2-4 μm across, brown pigmented, walls gelatinized, septa with clamps but difficult to see (Wasser and Weis, 1999, Wasser, 2011, Kosakyan et al., 2014).

Pleurotus eryngii (DC: Fr.) Quél.

Description P. eryngii is preferred over other species of Pleurotus mushrooms species because of its consistency, longer shelf life, pleasant aroma, and culinary qualities. Many investigations conform that P. eryngii is distinguished by its nutritional value and chemical composition (Singh et al., 2020). It is established that P. eryngii is a rich source of ergothioneine, which is an excellent in vivo antioxidant and protects cells from oxidative damage (Estrada et al., 2009). Various studies show that P. eryngii is rich in polysaccharides (mainly β-glucans), proteins, amino acids, and minerals (Gregori et al., 2007). Also, it contains a good amount of ergosterol which can be converted to vitamin D2 through UV irradiation. It was reported, that produced submerged mycelial biomass has an enhanced level of micronutrients (Ca, Co, Fc) and vitamin D2 as compared to its fruiting bodies reported in the literature. Beside this, the dried mycelia is a good source of ergosterol, proteins, crude fat, carbohydrates, crude fiber, minerals, and micro-elements (Singh et al., 2020). Due to rich chemical composition P. eryngii confer a variety of health benefits such as immunomodulatory, anti-inflammatory. P. eryngii is a significant source of lovastatin, also known as monocline k, which reduces blood cholesterol, has anti-inflammatory, anticoagulation, antioxidative, antifungal, and anticarcinogenic effects (Golak-Siwulska et al., 2018). P. eryngii contains the highest total glucan concentrations among Pleurotus spp. Polysaccharides extracted from P. eryngii mycelial biomass have demonstrated hepatoprotective and antioxidant effects. Moreover, P. eryngii is found to be a rich source of important major minerals like K, Mg, Ca, Fe. It contains a significant number of secondary metabolites like phenolic acids, polyphenols, and lovastatin being considered as compounds having a wide influence on human body functions (Zieba et al., 2020). Therefore, P. eryngii fruiting bodies and mycelium can be used as a raw material for diet supplements production and for pharmaceutical industry.

Characteristics. Vegetative mycelium in pure culture: MEA; PDA, optimal growth temperature 27° C. on MEA or PDA, appropriate growth pH 6.0. The average period of mycelium development in P. eryngii is ˜15-27 d. Teleomorph: The sporocarp reaches a size of up to 15 cm. It is composed of cream-colored to a light brown hat with a lamellar type hymenophore going down and the thick, soft stalk, which is edible in contrast to other Pleurotus spp. Pileus cochleariform to flabelliform, margin in rolled, convex; surface white, with creamcolored spots, with cracks and indistinct scales; flesh white, thick. Gills white, crowded, decurrent, 1-2 mm in width. Stipe lateral, solid, white, attenuate downwards. Spore (9) 10-14×(4.2) 5-6 μm, Q=2.0-2.5 (Q=2.2+0.21), oblong-elliptic to elliptic, colorless and hyaline. Basidia 30-45 (50)×7-9 μm, clavate, hyaline, thin-walled, four-spored (Zhao et al., 2016, Zieba et al., 2020).

Tremella fuciformis Berk.

Description T. fuciformis is low in energy and lipid, but rich in protein, polysaccharides and dietary fiber. It contains a variety of minerals, trace elements and vitamins. Plenty of bioactive substances are discovered in T. fuciformis, including fatty acids, proteins, enzymes, polysaccharides, phenols, flavonoid, dietary fiber and trace elements. Despite this, polysaccharides are the main bioactive compound isolated from fruiting body and mycelium of T. fuciformis. Natural polysaccharides derived from this mushroom are viewed as ideal ingredients for healthy foods and pharmaceutics (Wang et al., 2018 a,b). They have many bioactivities such as improving lowering blood sugar and blood fat, antithrombosis and antimutagenicity, antitumor and immunomodulatory, anti-oxidation and anti-aging, repair brain memory impairment, antiulcer and anti-inflammatory, hypoglycemic and hypocholesterolemic activities (Wen et al., 2016, Wu et al., 2019). The polysaccharides of T. fuciformis, can be considered as a substance to use successfully in food, cosmetics, medicines, and health care products.

Characteristics. It is whitish or nearly transparent, and fairly large (up to about 7 cm across) and it features graceful gelatinous lobes rather than the glob-like blobs that typify so many other jelly fungi. Optimal growth temperature 25° C. Telcomorph: Primordia are developed approximately 16 days (15-20 days) after the appearance of the white mycelial globules. Fruiting body is gelatinous but fairly firm; composed of graceful lobes; translucent whitish; up to about 7 cm across and 4 cm high; surface smooth and shiny. Spore print white. Spores 7-14×5-8.5μ; ovoid; smooth; often germinating by repetition. Basidia 4-spored; becoming longitudinally 4-septate (cruciate) with maturity; 11-15.5×8-13.5μ, with sterigmata up to 50×3μ. Clamp connections present (Horn et al., 1993, Binion et al., 2008).

Trametes versicolor (L.: Fr.) Lloyd

Description. In addition to the major macromolecules such as (proteins, carbohydrates, and lipids) and minerals, the mushroom is known to contain potential pharmacologically active secondary metabolites belonging to low molecular weight compounds. Methanol ethanol, and hot water extracts obtained from T. versicolor are generally the richest sources of total phenolic compounds and flavonoids such as flavones, flavonols, flavanone, flavanols, biflavonoids, isoflavonoids. The biological activities of the water extracts of C. versicolor, especially in the antioxidant area, must, therefore, account for the cumulative effects of the phenolic compounds. Beside this, T. versicolor is a great source of carbohydrates, proteins, amino acids, and minerals. The main bioactive components of T. versicolor are the polysaccharopeptides, which are isolated from the mycelium as well as fermentation broth. As a commercial product, polysaccharopeptides from T. versicolor (protein-bound polysaccharides/protcoglucans PSP-polysaccharide peptide, and PSK-Krestin), are available. PSP and PSK have been approved as medicines primarily as adjuvants in cancer therapy (Habtemariam et al., 2020). It was demonstrated various evidence of T. versicolor extracts efficacy through clinical trials, which reported an impressive result showing a significant survival advantage when compared with standard conventional anticancer (breast cancer, gastric cancer, hepatocellular carcinoma, or colorectal cancer) agents alone (Habtemariam et al., 2020). It is also worth noting that C. versicolor polysaccharides have been shown to ameliorate obesity (Li et al., 2019) or experimental diabetes in rodents. As anti-inflammatory agents, they further showed their benefit in inflammatory bowel disease or induction of analgesia (Gong et al., 1998). Their health protective effect was also proven through experimental models' diabetic cardiomyopathy (Wang et al., 2019a). Therefore, the health-beneficial effect of T. versicolor is established and proven as a result of numerous investigations.

Characteristics [CBS 147000]. Vegetative mycelium in pure culture: MEA; PDA, optimal growth temperature 27° C. on MEA or PDA, appropriate growth pH 6.0. Mycelial colony white, cream downy to cottony-woolly or floccose, becoming felty. Advancing zone appressed; hyphae rather distant. Reverse bleached. Chlamidospores typically present, sometimes arthroconidium-like, 4.5-9×25.6 μm. Clamp connections are often sprouting. Teleomorph: Pileus up to 10 cm across; only a few mm thick; flexible when fresh; circular, semicircular, bracket-shaped, or kidney-shaped; densely hairy or velvety, often with alternating zones of texture; with concentric zones of white, brown, cinnamon, and reddish brown (but highly variable in color and sometimes with other shades). Pores whitish to pale grayish; not bruising; pores tiny (4 or more per mm); tubes up to 3 mm deep. Context insubstantial; whitish; tough and leathery. Spores print whitish. Basidiospores 5-6×1.5-2 μm, smooth, cylindric. Basidia with 2-4 sterigmata, clavate, 15-20×5-6.0 μm. Cystidia not observed. Hyphal system trimitic, generative hyphae thin walled, 1.5-3.5 μm across, septa with clamps; skeletal hyphae thick-walled, 2.5-5 μm across; binding hyphae strongly branched and curved, thick-walled, 2-5 μm across (Tura et al., 2011, Sharma, 2012, Ryvarden and Melo, 2014).

### Example 3. Mushroom Inoculum Preparation and Biomass Scale Up Production in Submerged Conditions

The medicinal mushrooms used in the present study were Agaricus brasiliensis, Auricularia aricula-judae, Coprinus comatus, Cordyceps militaris, Flammulina velutipes, Ganoderma lucidum, Ganoderma tsugae, Grifola frondosa, Hypsizygus marmoreus, Hericium erinaceus, Inonotus obliquus, Lentinus edodes, Pleurotus ostreatus, Pleurotus eryngii, Tremella fuciformis, and Trametes versicolor. Pure cultures of these species were maintained on the malt extract agar slants contained tubes and Petri dishes and stored at 4° C. Mushroom inoculums were grown in 2 L flasks (contained 1 L nutrient medium), on a rotary shaker (refrigerated shaker laboratory incubator, MRC, Israel) at 150 rpm and 27° C. for 7-10 days in synthetic medium containing (g/l of distilled water): glucose, 15.0; peptone, 3.0; KH₂PO₄, 1.0; MgSO₄·7H₂O, 0.5; yeast extract, 3.0. Initial pH was adjusted to 6.0 prior to sterilization. Mushroom inoculums (mycelial pellets) were homogenized using 1 L volume a Warning laboratory blender (Waring Products Division, Torrington, CT, USA) three times for 20 seconds with 20 seconds intervals and homogenized mycelial suspension (10%) was used to inoculate nutrient medium in fermenter. At the end of cultivation 1 ml of the samples from each culture were taken for microscopic observation of inoculum purity.

To produce mushroom mycelial biomass, the submerged fermentation was performed in 10 L laboratory scale fermenter (Bioflo 200, New Brunswick Scientific, USA) with three Rushton impellers and 7 L working volume, equipped with instrumentation for the measurement and control of agitation, aeration, temperature, pH, and foam. 7 L of nutrient medium was used for mushroom cultivation in 10 L fermenter. The following basal nutrient medium contained (g/l of distilled water): glucose, 20; peptone, 3; yeast extract, 3; KH₂PO₄, 0.8; K₂HPO₄, 0.2; MgSO₄·7H₂O, 0.5. Initial parameters of cultivation were as follow: temperature 27° C., agitation 150 rpm, aeration 0.3 v/v/min, pH 6.0. KOH (2M) and 3% HCl were used for pH control.

### Results

The data of yield of biomass after submerged cultivation of medicinal mushrooms are presented in Table 1. 10% of 7 days old inoculums from each tested mushroom strains were inoculated on glucose contained synthetic mediums. Nutrient medium used appeared as a favorable condition for all tested mushroom growth and biomass accumulation. The yield of mycelial biomass after 7-14 days of mushroom cultivation in identical culture conditions varied from 4.8 g/l to 10.2 g/l (Table 1). It was observed a positive effect of glucose concentration used (20 g/l) on biomass accumulation by most tested mushroom strains. The highest biomass accumulation was reached by Flammulina velutipes (10.2 g/l), and Tremella fuciformis (10.0 g/l) followed by Cordyceps militaris (9.4 g/l), Pleurotus ostreatus (9.1 g/l), and Pleurotus eryngii (9.7 g/l). In comparison, Lentinus edodes and Grifola frondosa appeared as a most pure producers of mycelial biomass and accumulated only 3.8 g/l and 4.7 g/l after fourteen and eleven days of cultivation, respectively.

TABLE 1
Yield of biomass after submerged cultivation
of medicinal mushrooms

| | Yield of | Cultivation |
|---|---|---|
| Mushroom | biomass (g/l) | period (days) |
| Agaricus brasiliensis | 6.0 | 8 |
| Auricularia aricula-judae | 5.8 | 10 |
| Coprinus comatus | 5.9 | 7 |
| Cordyceps militaris | 9.4 | 13 |
| Flammulina velutipes | 10.2 | 10 |
| Ganoderma lucidum | 6.6 | 7 |
| Ganoderma tsugae | 6.4 | 7 |
| Grifola frondosa | 4.7 | 11 |
| Hypsizygus marmoreus | 6.2 | 8 |
| Hericium erinaceus | 7.0 | 7 |
| Inonotus obliquus | 8.9 | 8 |
| Lentinus edodes | 3.8 | 14 |
| Pleurotus ostreatus | 9.1 | 7 |
| Pleurotus eryngii | 9.7 | 7 |
| Tremella fuciformis | 10.0 | 11 |
| Trametes versicolor | 5.4 | 8 |

### Example 4. Preparation of Hot Water Extracts from Mushroom Mycelial Biomass

At the end of cultivation, mushroom biomass was separated from the culture liquid by filtration and mycelium was dried at 50° C. for 24 hours. Dried mycelium biomasses were milled to fine powder using commercial grade electric grinder (MRC, Israel) and combined in different proportion, and appropriate numbering was used for each combination (Table 2).

Mixtures of mycelial powders were used for the extraction of bioactive compounds. For extraction, distilled water was used. The procedure was as follow: the dry powder of mycelial biomass was extracted for 3 h with d H₂O (1 g/10 mL proportion) at 80° C. (using a water bath) (Shaking Water Bath, MRC, Israel). After extraction, insoluble solid compounds were separated by centrifugation at 6000 rpm at 4° ° C. for 15 min followed by filtration through the Whatman® filter paper N 1. Filtrates (supernatant) was evaporated using vacuum evaporator and finally dried at 40° C. in air forced laboratory oven (MRC, Israel).

### Results

The extraction of soluble compounds from dried and milled to a fine powder submerged mushroom mycelia was performed with hot water. Significant differences in yield among the extracts received from different mushroom species and their various combinations (Spl-1-Spl-15) were revealed and the results are shown in Table 3.

TABLE 2
Dried mycelium biomass combinations prepared and tested*

| Name | Content |
|---|---|
| Spl-1 | Lentinus edodes (33%), Coprinus comatus (33%), Tremella fuciformis (33%) |
| Spl-2 | Lentinus edodes (25%), Ganoderma lucidum (25%), Flammulina velutipes (25%), |
| | Trametes versicolor (25%) |
| Spl-3 | Inonotus obliquus (50%), Cordyceps militaris (25%), Ganoderma lucidum (25%) |
| Spl-4 | Lentinus edodes (25%), Pleurotus eryngii (25%), Grifola frondosa (25%), |
| | Trametes versicolor (25%) |
| Spl-5 | Pleurotus ostreatus (40%), Ganoderma lucidum (15%), Auricularia aricula-judae (15%) |
| | Trametes versicolor (15%), Lentinus edodes (15%) |
| Spl-6 | Pleurotus ostreatus (30%), Ganoderma lucidum (20%), Auricularia aricula-judae (20%) |
| | Trametes versicolor (15%), Lentinus edodes (15%) |
| Spl-7 | Trametes versicolor (20%), Hypsizygus marmoreus (20%), Flammulina velutipes (20%) |
| | Ganoderma lucidum (20%), Lentinus edodes (20%) |
| Spl-8 | Inonotus obliquus (30%), Cordyceps militaris (30%), Ganoderma lucidum (40%) |
| Spl-9 | Hericium erinaceus (40%), Ganoderma lucidum (30%), Cordyceps militaris (30%) |
| Spl-10 | Cordyceps militaris (50%), Ganoderma lucidum (25%), Ganoderma tsugae (25%) |
| Spl-11 | Inonotus obliquus (50%), Lentinus edodes (50%) |
| Spl-12 | Hericium erinaceus (75%), Ganoderma lucidum (25%) |
| Spl-13 | Lentinus edodes (40%), Cordyceps militaris (15%), Agaricus brasiliensis (15%), |
| | Coprinus comatus (15%), Pleurotus ostreatus (15%) |
| Spl-14 | Coprinus comatus (40%), Ganoderma lucidum (20%), Agaricus brasiliensis (20%), |
| | Tremella fuciformis (20%) |
| Spl-15 | Cordyceps militaris (20%), Grifola frondosa (20%), Tremella fuciformis (20%), |
| | Agaricus brasiliensis (20%), Ganoderma lucidum (20%) |

*Spl-5 and Spl-6, as well as Spl-3 and Spl-8 demonstrate the same mushroom combinations but in different proportions.

TABLE 3
Yield of hot water extracts prepared from dried
submerged cultivated medicinal mushroom mycelia

| | Extraction yield |
|---|---|
| Sample | (% dry weight) |
| Spl-1 | 34.8 |
| Spl-2 | 57.3 |
| Spl-3 | 38.4 |
| Spl-4 | 39.4 |
| Spl-5 | 39.2 |
| Spl-6 | 42.7 |
| Spl-7 | 32.4 |
| Spl-8 | 41.2 |
| Spl-9 | 39.0 |
| Spl-10 | 43.8 |
| Spl-11 | 37.5 |
| Spl-12 | 34.5 |
| Spl-13 | 48.8 |
| Spl-14 | 37.6 |
| Spl-15 | 52.5 |

### Example 5. Determination of Crude Fat and Proteins Content, and Soluble and Total Polysaccharides in Hot Water Extracts

### Methods

Proximate composition of crude ash, crude fat, crude protein, and carbohydrate contents were measured according to the method of American Association of Cercal Chemists (AACC International, 2000). The energy value was calculated using the formula: Energy (calories)=9×crude fat+4×crude protein+4×(total polysaccharides−soluble polysaccharides).

### Results

Nowadays, medicinal mushrooms are recognized as a healthy food and a great source of many essential nutritional compounds with high nutritional value, as they are important sources of proteins (mainly essential amino acids), vitamins, minerals, dictary fibers (especially β-glucan), and phenolic compounds. The proximate chemical composition, including the soluble and total polysaccharides, crude protein, crude fat, ash, and total energetic contributions of hot water extracts from different mixtures of mushroom mycelial biomass dry powders were assessed, and the results are presented in Table 4. Compositional study showed that most of the tested samples were generally rich in protein, and carbohydrates, but low in crude fat. Comparatively different compositions of tested extracts from combinations of medicinal mushroom species were determined. It should be noted that richness of medicinal mushrooms with protein and carbohydrate contents and low-fat levels directly make them nutritionally rich.

### Crude Fat Content

According to the WHO (2020), total fat intake for humans should be less than 30% of the total energy intake to prevent unhealthy weight gain in adults and the intake of saturated fats should be less than 10% of the total energy intake to reduce the risk of developing noncommunicable diseases. Thus, to obtain a balanced diet, the consumption of mushrooms has attracted attention of consumers. Table 4 shows that the crude fat content in all tested samples generally were quite low, which was expected because testing was carried out not in fungal biomass but in extracts obtained from different combinations of mushroom biomass. It was observed that crude fat content was basically similar to each other in all samples and varied from 0.14±0.05% (Spl-4) to 1.07±0.34% (Spl-2). In samples Spl-8, Spl-9, and Spl-10, crude fat content was strictly similar and showed 0.32±0.17%. 0.33±0.10% and 0.31±0.10%, respectively. Similar to this, samples Spl-1. Spl-3, Spl-5 and Spl-6 demonstrate slightly high but also similar to each other crude fat content and reached 0.66±0.12%, 0.58±0.09%, 0.53±0.23%, and 0.62±0.52%, respectively. In the rest of the samples, the content of crude fat was in trace amount (Spl-13-0.26±0.01% and Spl-14-0.21±0.10%), or not found at all (samples Spl-11, Spl-12, Spl-15). In the frame of several studies, it was investigated proximate chemical composition of edible/medicinal mushrooms. It was confirmed that different mushroom species contain comparatively significant amount of crude fat content (Cohen et al., 2014). Table 4 shows that amount of crude fat was significantly lower (at list 2-3 folds) in tested samples to compare to the literature data. Therefore, low crude fat content in samples can be considered as a positive aspect in terms of health improvement.

### Crude Proteins

One of the major chemical components of mushrooms are proteins which are an essential macronutrient to human body growth and maintenance due to its important physiological functions, such as vital performance of hormones and enzymes action. Recently, proteins of fungal origin have gained the attention from scientific community, due to its high nutritional values associated to the rich level of essential amino acids when compared to vegetables (Bach et al., 2017). It was reported that the protein contents of mushrooms are affected by several factors, namely, the type of mushroom, the stage of development, and the amount of nitrogen available. Table 4 shows the crude protein content in the 15 different samples (extracts) obtained from the various combinations of mushroom mycelial powders. The sample containing the lowest protein contents were Spl-2 (1.97±0.40%), Spl-4 (4.18±0.56%) and Spl-10 (1.86±0.09%). The highest protein content was recorded for Spl-1 (4.87±3.68%) and Spl-14 (5.37±0.40%). In all other samples crude protein content was in the range 2.39±0.66%-3.90±0.26%. As expected, the crude protein content in each one of the extracts is much lower than the amount of crude protein in the biomass, because the extraction process was conducted at 80° C. and consequently a significant amount of the protein was denatured. Nevertheless, hot water extraction was justified because it is known that extraction under high temperature condition results in the separation of many bioactive compounds and secondary metabolites from the mushroom biomass.

### Polysaccharides

Mushrooms are well known producers of many biologically active substances. Among these bioactive compounds, polysaccharides (carbohydrate polymers) with various activities are the main component for the bioactivities of several medicinal mushroom species. The diverse activities displayed by polysaccharides include antitumor, immunomodulatory, anti-inflammatory, antiviral, antioxidative, hypoglycemic, and hepatoprotective effects (Wasser 2014, Vetvicka et al., 2019). Mushroom polysaccharides mainly composed of (1→3), (1→6)-α/β-glucans, glycoproteins and water soluble heteropolysaccharides. Glucan is a well-studied polysaccharide and can be produced by different mushroom species with various terms, such as lentinan from Lentinus edodes, ganoderan from Ganoderma lucidum, grifolan from Grifola frondosa, schizophyllan from Schisophyllum commune, and polysaccharide-K (PSK, Krestin) from Trametes versicolor. Total and soluble polysaccharides were evaluated in 15 tested samples and results are presented in Table 4. Firstly, it must be noted that no differences were revealed between soluble and total polysaccharide contents, which indicates the fact that polysaccharides extracted from biomass are soluble compounds. The yield of polysaccharides was strongly dependent on the species and percentage combinations of tested mushrooms. Highest content of total polysaccharides was determined in sample Spl-2 extract (58.46±4.15%) obtained from the combination of 4 mushrooms mycelial powders L. edodes, G. lucidum, F. verutipes, and T. versicolor in equal concentrations (25% each). Lowest content of polysaccharides was observed in sample Spl-7 and reached only 4.73±1.33% although combinations of 5 mushroom species were used, T. versicolor (20%), H. marmoreus (20%), F. velutipes (20%) G. lucidum (20%), and L. edodes (20%), respectively. It should be noted that samples Spl-3 and Spl-8 were prepared using the same mushroom species, but in different concentrations, Spl-3: I. obliquus (50%), C. militaris (25%), and G. lucidum (25%); and Spl-8: I. obliquus (30%), C. militaris (30%), and G. lucidum (40%), respectively. The content of polysaccharides in the sample Spl-8 (26.07±0.7%) was almost 2-fold higher to compare to Spl-3 (14.96±1.48%). It can be assumed that this is especially due to the high concentration of G. lucidum (40%) in Spl-8. Beside this, also the similar mushroom species were used for preparation of samples Spl-5: P. ostreatus (40%), G. lucidum (15%), A. aricula-judae (15%) T. versicolor (15%), and L. edodes (15%); and Spl-6: P. ostreatus (30%), G. lucidum (20%), A. aricula-judae (20%) T. versicolor (15%), and L. edodes (15%) where three mushroom mycelial powders out of five existed in different concentrations, but this did not have a significant influence on the content of polysaccharides, and only a small difference was observed between samples (Spl-5 26.88±7.64% and Spl-6 21.74±6.76%). Significantly low content of polysaccharides was observed also in the sample Spl-12 and Spl-14, 13.37±4.98% and 10.23±1.84% respectively. Both samples contained G. lucidum mycelial powders almost similar concentrations (25%-20%, respectively), but this did not have a significant impact on the polysaccharide contents in tested extracts. In all other samples, polysaccharide content was in the range 17.10±6.87%-29.57±5.92%. Finally, it can be concluded that submerged fermentation is a promising approach for obtaining of polysaccharide-containing extracts, which could be considered as a source of potentially natural pharmaceutical products.

### Ash Content

Ashing of samples was conducted at 550-600° C. for 3 h. It is known that ash in edible/medicinal mushrooms generally ranges from 5-15 g/100 g dry matter with the major minerals constituting about 56-70% of the total ash content. All tested samples demonstrate low and similar ash content and varied from 2.83±0.04% to 4.22±0.05% (Table 4).

TABLE 4
Proximate composition of medicinal mushroom extracts

| | | | | Soluble | Soluble | |
|---|---|---|---|---|---|---|
| | | | Crude | polysaccharides | polysaccharides + | |
| Sample | Ash (%) | Crude fat (%) | protein (%) | (%) | reducing sugars (%) | (%) |
| Spl-1 | 4.22 ± 0.05^{A} | 0.66 ± 0.12^{B} | 4.87 ± 0.66^{D} | 14.29 ± 3.68^{BC} | 14.30 ± 3.678^{BC} | 25.73 |
| Spl-2 | 3.55 ± 0.05^{EF} | 1.07 ± 0.34^{A} | 1.97 ± 0.40^{A} | 58.44 ± 4.15^{G} | 58.46 ± 4.15^{G} | 25.70 |
| Spl-3 | 3.23 ± 0.02^{I} | 0.58 ± 0.09^{A} | 3.90 ± 0.26^{BC} | 14.94 ± 1.48^{BC} | 14.96 ± 1.48^{BC} | 21.09 |
| Spl-4 | 4.00 ± 0.40^{B} | 0.14 ± 0.05^{D} | 4.18 ± 0.56^{C} | 17.11 ± 6.87^{BCD} | 17.10 ± 6.87^{BCD} | 18.22 |
| Spl-5 | 3.52 ± 0.05^{FG} | 0.53 ± 0.23^{BCD} | 3.56 ± 0.52^{BC} | 26.86 ± 7.64^{F} | 26.88 ± 7.64^{F} | 19.28 |
| Spl-6 | 3.64 ± 0.05^{DE} | 0.62 ± 0.52^{BC} | 3.37 ± 0.23^{B} | 21.73 ± 6.76^{CDEF} | 21.74 ± 6.76^{CDEF} | 19.34 |
| Spl-7 | 3.62 ± 0.05^{EF} | 0.18 ± 0.06^{CD} | 3.80 ± 0.31^{BC} | 4.71 ± 1.33^{A} | 4.73 ± 1.33^{A} | 17.01 |
| Spl-8 | 3.10 ± 0.04^{J} | 0.32 ± 0.17^{BCD} | 2.39 ± 0.66^{A} | 26.05 ± 2.46^{EF} | 26.07 ± 2.457^{EF} | 12.78 |
| Spl-9 | 3.39 ± 0.07^{H} | 0.33 ± 0.10^{BCD} | 3.50 ± 0.07^{BC} | 27.67 ± 3.15^{F} | 27.69 ± 3.15^{F} | 17.25 |
| Spl-10 | 2.83 ± 0.04^{K} | 0.31 ± 0.10^{BCD} | 1.86 ± 0.09^{A} | 28.58 ± 6.01^{F} | 28.59 ± 6.01^{F} | 10.45 |
| Spl-11 | 3.74 ± 0.04^{D} | — | 3.38 ± 0.26^{B} | 18.01 ± 4.13^{BCDE} | 18.02 ± 4.13^{BCDE} | 13.73 |
| Spl-12 | 3.83 ± 0.04^{C} | — | 2.10 ± 0.49^{A} | 13.36 ± 4.98^{BC} | 13.37 ± 4.98^{BC} | 8.54 |
| Spl-13 | 3.71 ± 0.07^{D} | 0.26 ± 0.01^{BCD} | 3.82 ± 0.46^{BC} | 29.55 ± 5.92^{F} | 29.57 ± 5.92^{F} | 15.28 |
| Spl-14 | 3.37 ± 0.10^{H} | 0.21 ± 0.10^{CD} | 5.37 ± 0.40^{D} | 10.22 ± 1.85^{AB} | 10.23 ± 1.84^{AB} | 23.65 |
| Spl-15 | 3.44 ± 0.09^{GH} | — | 3.67 ± 0.16^{BC} | 24.86 ± 1.00^{DEF} | 24.87 ± 1.00^{DEF} | 14.92 |

### Total Energy Contribution

Significant differences were observed in all energy values of corresponding samples and the results are shown in Table 4. The energy values of the studied samples were low and dependent on the mushroom species combinations and their concentrations. The total energy values examined varied from 8.54 cal/100 g to 25.73 cal/100 g.

### Example 6. Content of Ergothioneine and γ-Aminobuthiric Acid (GABA)

### Determination of Ergothioneine

The ergothioneine content was analyzed following the method of Dubost et al., (2006) with some modification. Freeze-dried mushroom extracts (1 g) were added to 20 mL solution (10 mmol/L 1,4-dithiothreitol, 100 mmol/L betaine, 100 mmol/L 2-mercapto-1-methylimidazole in 700 mL/L ethanol) and vortexed for 90 s. After 4 mL of 10 g/L sodium dodecyl sulphate solution was added and the mixtures were centrifuged at 25° C. and 3000 g for 10 min. The supernatant was then rotary evaporated at 40° C. to 5 mL and filtered through a 0.45 mm CA nonsterile filter. The HPLC system consisted of a Hitachi L-2130 pump, a Rheodyne 7161 injector, a 20-mL sample loop, a Hitachi L-2455 Diode Array Detector, and a Luna 5 m PFP (2) 100 A column (4.6 250 mm, Phenomenex). The mobile phase was 500 mmol/L sodium phosphate in water with 30 mL/L acetonitrile and 1 mL/L tricthylamine adjusted to a pH of 7.3 with at a flow rate of 1 mL/min and UV detection at 254 nm. Ergothioneine was quantified by the calibration curve of the authentic standard (Sigma).

### Determination of γ-Aminobutyric Acid (GABA)

Freeze-dried extracts (500 mg) were shaken with 50 mL of 0.1 mol/L HCl solution for 45 min at ambient temperature and filtered through Whatman No. 4 filter paper. The filtrates were then passed through a filter unit (13 mm, Lida), and filtered using 0.45 m CA non-sterile filter. This filtrate was mixed with o-phthalaldehyde reagent (Sigma, St. Louis, MO, USA) in an Eppendorf tube, shaken to facilitate derivatization and then immediately injected onto HPLC. The HPLC system consisted of a Shimadzu LC-10AT pump (Shimadzu, Tokyo, Japan), a Rheodyne 7161 injector, a 20-mL sample loop, a Hitachi FL-Detector L-2455 with fluorescence excitation at 340 nm and emission at 450 nm, and a chromolith RP18e column (4.6 100 mm, Merck, Darmstadt, Germany). The mobile phases were A, 50 mmol/L sodium acetate (pH 5.7) containing 5 mL/L tetrahydrofuran; B, deionized water; and C, methanol (Mau et al., 1997). The gradient was A: B: C 83:0:17 to 33:0:67 for 0e37 min, 0:33:67 for 37e40 min, and 0:100:0 for 40c43 min at the flow rate of 1.2 mL/min. GABA was quantified by the calibration curve of the authentic standard (Sigma).

### Results

Medicinal mushrooms are great source of secondary metabolites, including GABA and ergothioneine, which have the positive advantages for human health. GABA is known as a major inhibitory neurotransmitter in vertebrate brain. Perturbation of GABAergic signaling has been implicated in numerous neurological disorders, including epilepsy, Parkinson's disease, and Huntington's disease (Johnston et al., 2016). GABA is well known as the major inhibitory neurotransmitter in the mammalian central nervous system. It was reported to play vital roles in modulating synaptic transmission, promoting neuronal development and relaxation, and preventing sleeplessness and depression (Nuss, 2015). Notably, various biological activities of GABA were documented due to anti-hypertension, anti-diabetes, anti-cancer, antioxidant, anti-inflammation, anti-microbial, and anti-allergy. Moreover, GABA was also reported as a protective agent of liver, kidney, and intestine against toxin-induced damages (Ngo and Sang, 2019). Beside this, the protection and hypotensive actions of some food products containing GABA were shown in hypertensive patients (Tanaka et al., 2009). Ergothioneine is a unique sulfur-containing amino acid that cannot be synthesized by humans but has potent antioxidant activities. It is available only from certain dietary sources, specifically mushrooms fruiting bodies as well as mycelial biomass are primary source of ergothioneine containing from 0.4 to 2.0 mg/g (dry weight) (Ey et al., 2007). It was reported that, King oyster, maitake, oyster, and shiitake mushrooms contain the highest amounts of ergothioneine (Dubost et al., 2006). Ergothioneine is not synthesised by plants and animals who acquire it via the soil and their diet, respectively. ERG seems to have strong cytoprotective status, and its concentration is lowered in a number of chronic inflammatory diseases. It has been passed as safe by regulatory agencies, and may have value as a nutraceutical and antioxidant more generally (Borodina et al., 2020). Moreover, ergothioneine has been widely used as a “cosmeceutical” due to having cosmetic benefits (Pérez-Sánchez et al., 2018), since much skin damage is caused by UV-mediated reactive oxygen species production; indeed, ERG is known as a skin protectant (Borodina et al., 2020).

Bioactive compounds such as GABA and ergothioneine, quantified in 15 samples/extracts and the results are given in Table 5. Among all tested samples GABA was found only in 8 of them and varied from 0.16±0.03 mg/g to 4.97±0.83 mg/g. Samples Spl-2, Spl-4, and Spl-6 contained the highest amounts of GABA (4.97±0.83, 5.69±0.41, and 3.76±0.80 mg/g, respectively), whereas samples Spl-3, and Spl-7 contained the slightly low amount of GABA (0.94±0.31, and 0.16±0.03 mg/g). In contrast to GABA, ergothioneine content was found in all tested samples/extracts in different amount. Highest content was demonstrated by mushrooms combination Lentinus edodes-33%, Coprinus comatus-33%, and Tremella fuciformis-33% (Spl-1) reached 0.61±0.03 mg/g and Lentinus edodes-25%, Pleurotus eryngii-25%, Grifola frondosa-25%, and Trametes versicolor-25% (Spl-4) reached 0.58±0.09 mg/g. In samples Spl-6, Spl-7, Spl-9, Spl-12, and Spl-14 demonstrate similar (basically equal) ergothioneine contents in the range 0.40±0.01-0.46±0.05 mg/g (Table 5). Lowes amount was found only in sample Spl-10 (0.03±0.01 mg/g). The results show that the content of ergothioneine was strongly dependent on the mushroom species used and their concentrations in combinations. At last, it can be concluded, that tested medicinal mushrooms are an abundant source of bioactive secondary metabolites, such as GABA and ergothioneine, and it is another reason to incorporate mushrooms and mushroom derived substances into the human diet or can be used as a source of pharmaceuticals.

TABLE 5
Ergothioneine and GABA content

| | Ergothioneine | |
|---|---|---|
| Sample | (mg/g) | GABA (mg/g) |
| Spl-1 | 0.61 ± 0.03 | 1.73 ± 0.72 |
| Spl-2 | 0.32 ± 0.02 | 4.97 ± 0.83 |
| Spl-3 | 0.23 ± 0.06 | 0.94 ± 0.31 |
| Spl-4 | 0.58 ± 0.09 | 5.69 ± 0.41 |
| Spl-5 | 0.33 ± 0.01 | 2.00 ± 0.21 |
| Spl-6 | 0.40 ± 0.04 | 3.76 ± 0.80 |
| Spl-7 | 0.40 ± 0.01 | 0.16 ± 0.03 |
| Spl-8 | 0.21 ± 0.02 | — |
| Spl-9 | 0.43 ± 0.05 | — |
| Spl-10 | 0.03 ± <0.01 | — |
| Spl-11 | 0.27 ± 0.041 | — |
| Spl-12 | 0.46 ± 0.05 | 1.55 ± 0.73 |
| Spl-13 | 0.37 ± 0.037 | — |
| Spl-14 | 0.40 ± 0.03 | — |
| Spl-15 | 0.35 ± 0.05 | — |

### Example 7. Amino Acid Analyses

### Materials and Methods

For determination of amino acid content, each extract was treated with 0.1 mol/L of HCl for 45 min at ambient temperature and filtered using a 0.45-μm PVDF membrane filter (Merck Millipore). The purified filtrate was mixed with o-phthalaldehyde reagent (Sigma-Aldrich, St. Louis, MO, USA) in an Eppendorf® tube (Eppendorf, Hauppauge, NY, USA), shaken to facilitate derivatization, and then immediately injected into HPLC. The HPLC system consisted of an L-7485 fluorescence detector (Hitachi) working at an excitation wavelength of 340 nm and emission wavelength of 450 nm, and a LiChrospher® 100 RP-18 column (4.6 mm×250 mm, i.d. 5 μm; Merck Millipore, Darmstadt, Germany). The mobile phase consisted of 50 mmol/L of sodium acetate at pH=5.7, containing 0.5% tetrahydrofuran (solvent A), deionized water (solvent B) and methanol (solvent C). The linear gradient clution was performed using A:B:C in a volume ratio from 80:0:20 to 33:0:67 at 0-38 min, then to 0:33:67 at 38-40 min, and to 0:100:0 at 40-43 min, with the flow rate of 1.2 mL/min. Each amino acid content was calculated based on the calibration curve of the respective standard (Sigma-Aldrich).

### Results

Amino acids are the fundaments of enzymes, receptors, antibodies, signaling molecules, hormones, and multiple other essential protein structures in all living organisms including human. Although there are hundreds of amino acids found in nature, only about 20 amino acids are needed to make all the proteins found in the human body and most other forms of life. Of these 20 amino acids, nine amino acids are essential: phenylalanine, valine, tryptophan, threonine, isoleucine, methionine, histidine, leucine, and lysine. Therefore, they must be supplied in the food. Failure to obtain enough of even 1 of the 10 essential amino acids, those that we cannot make, results in degradation of the body's proteins-muscle and so forth, to obtain the one amino acid that is needed. Unlike fat and starch, the human body does not store excess amino acids for later use the amino acids must be in the food every day. The very reason for the severe problems in humans with an essential amino acid deficiency is a reduced rate of protein synthesis in cells and tissues, particularly skeletal muscle (Hou and Wu, 2018). Amino acid composition is a reliable indicator of the nutritional value of food. Amino acids are the main constituents of functionally essential compounds that are found in mushrooms. Amino acid composition of mushrooms is reported to be comparable to animal proteins.

Table 6 shows categories and the content of amino acids in tested samples/extracts. Data received indicate, that in almost all the tested samples it was possible to determine 17 essential and nonessential amino acids: alanine, arginine, aspartic acid, cysteine, glutamic acid, glycine, histidine, isoleucine, leucine, lysine, methionine, phenylalanine, proline, serine, threonine, valine, and tryptophan. Whereas all essential amino acids were presented in most tested samples and these were dominated by phenylalanine and methionine, ranged 108.29-516.60 mg/g and 11.21-80.80 mg/g, respectively. Contents of methionine and phenylalanine in all samples were extremely high (Spl-1: phenylalanine—516.60 mg/g, methionine—44.40 mg/g; Spl-14: phenylalanine −393.36 mg/g, methionine—37.72 mg/g). Beside this, extremely big content of leucine and methionine were observed in the sample Spl-8 (88.05 mg/g and 80.80 mg/g, respectively). These higher yields are explained by the fact that content of amino acids was tested not in biomass but in hot water extracts obtained from biomass which facilitated the extraction and concentration of amino acids in the samples. Surprisingly, cysteine, histidine and proline did not detect in all tested samples. Furthermore, Spl-11 and Spl-13 were most pure samples in amino acid contents, whereas contained only 8 and 4 amino acids out of evaluated 17 (Table 6). It should be noted that the total content of essential amino acids in tested samples were higher to compare to the content of nonessential amino acids.

TABLE 6
Total amino acids composition

| | Spl-1 | Spl-2 | Spl-3 | Spl-4 | Spl-5 | Spl-6 | Spl-7 | Spl-8 |
|---|---|---|---|---|---|---|---|---|
| Ala | 4.23 | 5.09 | 4.75 | 6.26 | 2.23 | 4.78 | 3.68 | 9.13 |
| Arg | 6.09 | 4.99 | 6.07 | 6.46 | 3.54 | 6.34 | 5.94 | 13.75 |
| Asp | 1.10 | 1.14 | 1.95 | 0.21 | 1.59 | 0.18 | 2.51 | 31.92 |
| Cys | — | — | — | — | — | — | — | — |
| Glu | 2.86 | 4.84 | 2.10 | 2.24 | 1.69 | 2.06 | — | — |
| Gly | 0.35 | 0.04 | — | 0.02 | — | — | — | — |
| His | — | — | — | — | — | — | — | — |
| Isoleu | 5.74 | 6.44 | 3.34 | 3.95 | 2.68 | 2.91 | — | — |
| Leu | — | — | 55.65 | — | — | — | 54.65 | 88.05 |
| Lys | 12.45 | 16.63 | 10.51 | 20.77 | 21.26 | 17.70 | 12.32 | 6.67 |
| Met | 44.40 | 57.73 | 76.32 | 38.55 | 27.62 | 72.86 | 43.00 | 80.80 |
| Phe | 516.60 | 122.06 | 158.62 | 152.56 | 108.29 | 147.71 | 145.73 | 239.48 |
| Pro | — | — | — | — | — | — | — | — |
| Ser | 6.95 | 6.11 | 4.03 | 16.02 | 3.04 | 4.51 | 4.40 | 8.44 |
| Thr | 5.43 | 6.67 | 4.82 | 6.45 | 6.12 | 6.16 | 5.01 | 4.85 |
| Val | 1.73 | — | — | 1.14 | 1.25 | 0.34 | — | 0.31 |
| Try | 3.15 | — | — | — | — | — | — | 0.91 |
| Total | 611.08 | 231.74 | 328.16 | 254.63 | 179.31 | 265.55 | 277.24 | 484.31 |
| | | Spl-9 | Spl-10 | Spl-11 | Spl-12 | Spl-13 | Spl-14 | Spl-15 |
| | Ala | 1.95 | 3.82 | 1.06 | 4.02 | — | 3.34 | 3.18 |
| | Arg | 4.11 | 3.96 | — | 11.62 | — | 4.06 | 3.86 |
| | Asp | 5.84 | 12.12 | — | 5.17 | 0.15 | 2.33 | 1.41 |
| | Cys | — | — | — | — | — | — | — |
| | Glu | 1.40 | 1.37 | — | 1.68 | — | 1.62 | 1.17 |
| | Gly | — | — | 2.15 | — | — | — | — |
| | His | — | — | — | — | — | — | — |
| | Isoleu | — | — | — | — | — | — | — |
| | Leu | 20.93 | 38.93 | 16.30 | 41.60 | — | 61.46 | 57.98 |
| | Lys | 7.52 | 7.65 | 9.60 | 7.68 | 6.21 | 8.52 | 16.09 |
| | Met | 18.13 | 37.68 | 9.78 | 29.95 | 11.21 | 37.72 | 36.32 |
| | Phe | 229.96 | 295.79 | 139.51 | 426.46 | 359.14 | 393.36 | 267.01 |
| | Pro | — | — | — | — | — | — | — |
| | Ser | 1.28 | 0.54 | 0.65 | 2.76 | — | 1.56 | 0.13 |
| | Thr | 3.49 | 3.78 | 0.72 | 6.09 | — | 3.63 | 4.85 |
| | Val | 0.03 | — | — | — | — | — | — |
| | Try | 0.66 | 0.33 | — | 0.45 | — | 0.38 | 0.30 |
| | Total | 295.3 | 405.97 | 179.77 | 537.48 | 376.71 | 517.98 | 392.3 |

### Example 8. Evaluation of Total Phenolic and Flavonoid Content

### Determination of Total Phenols

Total phenols of extracts were determined according to the method of Jun et al. (2012) with minor modification. Each extract (500 mg) was dissolved in 80% (v/v) methanol (6 mL) using an ultrasonic bath with 40 kHz for 5 min, and then the volume was adjusted to 10 ml. Folin-Ciocalteu's phenol reagent (0.4 mL) was added to each sample (0.2 mL), and stood for 1 min. Then, 6 mL of 5 g/100 mL sodium carbonate solution was added to the mixture and stood for 1 h in dark place. The absorbance was measured at 760 nm using a spectrophotometer (U-1800, Hitachi Hightechnologies Co., Tokyo, Japan), with gallic acid used as a standard. The content of total phenols was calculated based on the calibration curve of gallic acid [the equation of standard curve: absorbance at 760 nm=0.0037 C_{gallic acid} (μg/ml)+0.0173. R²=0.9996]. Results were expressed as milligram of gallic acid equivalents (GAE) per gram of mushroom extract.

### Evaluation of Flavonoid Content

Each sample (100 mg) was dissolved in ethanol and an aliquot (500 μL) of the resulting solution was added to 1 ml of 5% sodium nitrite. After standing in the dark for 5 min, 100 μL of 10% aluminum chloride was added to the mixture. Again, after standing in the dark for 5 min, 1 ml of 5% sodium hydroxide and 800 μL of deionized water were added. Again, after standing in the dark for 15 min, the absorbance was then measured at 510 nm. Results were expressed as milligram of Rutin (RE) equivalents per gram of mushroom extract.

### Results

Total phenolic content. Among different bioactive compounds, phenolic compounds represent one of the major class of chemical substances found in mushrooms. Phenolic compounds are aromatic hydroxylated compounds with one or more aromatic rings and one or more hydroxyl groups. Phenolic compounds are a large group of secondary plant metabolites which play a major role in the protection of oxidation processes. It has been reported that phenolic compounds exhibit antioxidant activity in biological systems, acting as hydrogen donators, singlet oxygen quenchers, free radical inhibitors, peroxide decomposers, metal inactivators or oxygen scavengers (Sanchez, 2017). Numerous studies have conclusively demonstrated that phenolic compounds are present in all the mushrooms. These compounds can be pyrogallol, myricetin, caffeic acid, quercetin and catechin among others. Therefore, phenolics exhibit a wide range of biological effects including antioxidant, antibacterial, anti-inflammatory, anti-Alzheimer's effect and antihyperglycemic (Sanchez, 2017, Kim, 2020a).

The contents of total phenolic compounds in the mushroom extracts are shown in Table 7, with the results expressed as milligrams of gallic acid equivalents per gram (mg GAE/g). The results indicated that considerable amounts of total phenolic compounds were found in most mushroom extracts. It was shown that the total phenolic compounds content demonstrates significant differences between the solvents used for extraction. Garcia et al. (2020) determine that there were significant differences concerning the extraction solvent used, with the aqueous solvent provided a higher total phenolic compounds content than methanol. Highest and almost similar amount of total phenolic content was demonstrated by hot water extract samples Spl-3, Spl-6, Spl-12, and Spl-14 accumulated 18.83±0.21, 18.88±0.34, 18.3±0.21, and 18.35±0.23 mg GAE/g, respectively. The high concentration of phenolic content in the samples were most likely provided by Ganoderma lucidum, which was the only mushroom existed in the combinations of mycelial powders from which above mentioned samples/extracts were obtained. Several studies confirm, that G. lucidum is well known producer of different phenolic compounds and therefore it is proven to have significant antioxidant properties. Samples Spl-7, Spl-8, Spl-9, Spl-10, and Spl-11 showed also similar contents of total phenols and were in the range 17.3±0.62-17.97±0.09 mg GAE/g (Table 7). The results show that the total phenolic compounds found in all tested extracts. Therefore, they can be considered to possess significant antioxidant properties to inhibit lipid peroxidation, to scavenge free radicals and to chelate ferrous ions.

TABLE 7
Total phenolic compounds and flavonoid content

| | Phenols | Flavonoids |
|---|---|---|
| Sample | (mg GAE/g) | (mg RE/g) |
| Spl-1 | 16.71 ± 0.38^{EF} | 2.66 ± 0.14^{C} |
| Spl-2 | 16.42 ± 0.03^{F} | 1.50 ± 0.16^{EF} |
| Spl-3 | 18.83 ± 0.21^{A} | 3.16 ± 0.24^{BC} |
| Spl-4 | 16.63 ± 0.33^{F} | 2.89 ± 0.13^{C} |
| Spl-5 | 15.53 ± 0.23^{G} | 3.23 ± 0.21^{BC} |
| Spl-6 | 18.88 ± 0.34^{A} | 2.09 ± 0.22^{D} |
| Spl-7 | 17.35 ± 0.07^{CDE} | 3.63 ± 0.21^{B} |
| Spl-8 | 17.3 ± 0.62^{CDE} | 3.14 ± 0.28^{BC} |
| Spl-9 | 17.42 ± 0.29^{CD} | 1.98 ± 0.3^{DE} |
| Spl-10 | 17.50 ± 0.61^{C} | 4.34 ± 0.73^{A} |
| Spl-11 | 17.97 ± 0.09^{BC} | 3.63 ± 0.07^{B} |
| Spl-12 | 18.3 ± 0.21^{AB} | 2.68 ± 0.48^{C} |
| Spl-13 | 16.77 ± 0.76^{DEF} | 1.23 ± 0.04^{FG} |
| Spl-14 | 18.35 ± 0.23^{AB} | 3.19 ± 0.30^{BC} |
| Spl-15 | 16.11 ± 0.16^{FG} | 2.63 ± 0.66^{G} |

Total flavonoid content. Flavonoids (low-molecular-weight phenolic compounds) are an important class of natural products; particularly, they belong to a class of natural secondary metabolites having a polyphenolic structure, found in fruits, vegetables, and certain beverages. They have miscellaneous favorable biochemical and antioxidant effects. Flavonoids are associated with a broad spectrum of health-promoting effects and are an indispensable component in a variety of nutraceutical, pharmaceutical, medicinal, and cosmetic applications. Scientific evidence has strongly shown that regular intake of dietary flavonoids in efficacious amounts reduces the risk of oxidative stress and chronic inflammation-mediated pathogenesis of human diseases such as cardiovascular disease, certain cancers, and neurological disorders. Beside this, it is proven a broad and multi-potent pharmacological property of flavonoids, such as diabetes, allergies, and osteoporosis (Fernandes et al., 2017). Flavonoids also offer promising applications in the management of obesity- and inflammation-associated disorders as well as the control of infectious diseases, including COVID-19 (Vasantha, 2020). The prevention of some diseases is related to the strong antioxidant capacity of flavonoids. Through their antioxidant property, these compounds could potentially prevent free-radical-related injury. They are able to scavenge a wide range of reactive oxygen (ROS), nitrogen (NO) and chlorine species as well as inhibit the production of reactive species.

Mushrooms successfully can produce flavonoid compounds. Previously several reports have shown the presence of flavonoids (i.e., catechin, hesperetin, naringenin, naringin, formometin, quercetin, rutin, and kaempferol) in different mushroom species (Xiaokanga et al., 2020b). In the present invention, Table 7 contains the results of flavonoid profiles in tested wat water extract samples obtained from different combinations of medicinal mushrooms dry powders. Significant differences in the content of flavonoids were observed in extracts obtained from different combinations and varied from 1.23±0.04 (Spl-13) to 4.34±0.73 (Spl-10). Flavonoid contents significantly were dependent on percentage of mushroom powders in their combinations. Out of the 15 tested samples, 6 demonstrate similar flavonoid contents (Spl-3-3.16±0.24 mg RE/g, Spl-5-3.23±0.21 mg RE/g. Spl-7-3.63±0.21 mg RE/g. Spl-8-3.14±0.28 mg RE/g. Spl-11-3.63±0.07 mg RE/g, and Spl-14-3.19±0.30 mg RE/g). These extracts were obtained from combinations mushroom dry powders dominated by I. obliquus, C. militaris, and G. lucidum, which are known for their ability to synthesize polyphenolic compounds, including flavonoids. Lowest content of flavonoid compounds was showed only by 3 samples, Spl-2, Spl-9, and Spl-13, contained 1.50±0.16 mg RE/g. 1.98±0.3 mg RE/g, and 1.23±0.04 mg RE/g respectively (Table 7). The result indicated that tested medicinal mushroom species and extracts thereof have a promising antioxidant potential due to presence of flavonoids and phenolic compounds. It can be concluded that, because of presence of phenolic compounds/flavonoids in the mushroom extracts results in several biological activities, therefore, they strongly can be recommended as source of pharmaceutical products, rich in antioxidant compounds.

### Example 9. Antioxidant Properties of Extracts from Different Mycelial Biomass Combinations

Free radicals and reactive oxygen species (ROS), which are produced as side-products of several metabolic processes, can seriously harm cells through oxidation processes. ROS are chemically reactive molecules containing oxygen ions and peroxides. ROS including radical species such as hydroxyl (OH) and superoxide radical (O²) along with non-radical species hydrogen peroxide (H₂O₂) are generated during normal cellular metabolism. Increased ROS levels cause damage to lipids, proteins, and nucleic acids leading to physiological disability that often results in metabolic disorders of inflammation, aging, cancer, and hypertension. Cells can be protected against ROS-mediated damage via actions of substances called antioxidant compounds. The long-term presence of free radicals and reactive oxygen species accelerates aging and causes numerous illnesses. Pharmaceutical/dietary intake of antioxidants is an important approach and may contribute to oxidative homeostasis. Synthetic antioxidants, with the characteristics of low cost, high performance, and broad bioactivity, have long been in common usage in food, pharmaceuticals, and cosmetics. But, in recent years it became known that although synthetic antioxidants such as butylhydroxyanisole (BHA), butylhydroxytoluene (BHT), and tert-butylhydroquinone (TBHQ), ethoxyquin (EQ) effectively inhibit oxidation, have a toxic effect (a specially in high concentrations) causing damage of the body and could be responsible for different types of tumor and liver damage. Natural products generally offer the advantage of not inducing toxic effects. Therefore, in view of increasing risk factors of human to various deadly diseases, the search of new, effective, natural, safe, and multiple biological compounds with high antioxidants properties is a significant challenge. In addition, the sympathy of the population is directed to the use of natural products. Both polysaccharides and phenolic compounds have been documented as antioxidants produced by mushrooms and are therefore considered to be an excessive source of natural antioxidants having pharmaceutical potential. Phenolic compounds/flavonoids produced by mushrooms have been documented as antioxidants. At the same time, the phenolic extracts from many mushroom exhibit antimicrobial activity against wide range of pathogens and therefore can serve as a potential source of both antimicrobial and antioxidant compounds. Therefore, it is highly recommended to introduce mushrooms and mushroom derived substances directly into the diet to promote human health. Determination of in vitro antioxidant activity can be successfully carried out by several different methods such as DPPH scavenging activity, reducing power, chelating ability, hydroxyl radical scavenging activity, and 2,2′-azino-bis(3-ethylbenzothiazoline)-6-sulphonic acid (ABTS) scavenging activity. In present invention the complexity of samples/extracts required multiple radical scavenging assays in order to verify their antioxidant capacity. Therefore, the free radical scavenging activity of mushroom extracts was assessed by DPPH, and ABTS assays. All of them confirms the capable radical scavenging abilities of antioxidant compounds extracted from medicinal mushrooms.

The DPPH assay is considered a technically simple yet accurate method to preliminarily measure the antioxidant capacity of a mushroom extract, while ABTS assay was employed to measure the radical scavenging activity of those substances having lipophilic or hydrophilic properties, for instance, flavonoids and carotenoids. Moreover, reducing power and chelating of ferous ions were evaluated to represent a spectrum of antioxidant capacity of tested samples.

### Materials and Methods

Scavenging activity on DPPH radicals. Each mycelial extract (0.5-10 mg/ml) in methanol (4 ml) was mixed with 1 ml of a methanolic solution containing DPPH (Sigma) radicals, resulting in a final concentration of 0.2 mM DPPH. The mixture was shaken vigorously and left to stand for 30 min in the dark, and the absorbance was then measured at 517 nm against a blank (Shimada et al., 1992). The antioxidant activity in the samples were expressed as Trolox equivalents, using a calibration curve plotted with different amounts of Trolox (water-soluble analogue of vitamin E. Sigma Aldrich).

Scavenging activity on ABTS radicals. The experiments were carried out using an improved ABTS decolonization assay (Re et al., 1999). The ABTS radical cation (ABTS+) was produced by reacting 7 mM stock solution ABTS with 2.45 mM potassium persulphate (final concentration) and allowed the mixture to stand in the dark for at least 6 hours at room temperature before use. The ABTS+ solution was diluted to an absorbance 0.7±0.05 at 736 nm. Absorbance was measured 7 minutes after the initial mixing of different concentrations of the extracts. The ABTS+ decolonization capacity of the extracts was compared with the standard Trolox. A standard curve was prepared by measuring the reduction in the absorbance of ABTS+ solution at different concentration of Trolox over a period of 7 minutes.

Reducing power. The reducing power was determined according to the method of Oyaizu (1986). Each mycelial extract (0.5-10 mg/ml) in methanol (2.5 ml) was mixed with 2.5 ml of 200 mM sodium phosphate buffer (pH 6.6, Wako Pure Chemical Co., Osaka, Japan) and 2.5 ml of 1% potassium ferricyanide (Sigma Chemical Co., St. Louis, MO), and the mixture was incubated at 50° C. for 20 min. After 2.5 ml of 10% trichloroacetic acid (w/v, Wako) were added, the mixture was centrifuged at 200 g for 10 min. The upper layer (5 ml) was mixed with 5 ml of deionized water and 1 ml of 0.1% ferric chloride (Wako), and the absorbance was measured at 700 nm against a blank in a Hitachi U-2001 spectrophotometer. A higher absorbance indicates a higher reducing power. The antioxidant activity in the samples were expressed as Trolox equivalents, using a calibration curve plotted with different amounts of Trolox.

Chelating effects on ferrous (Fe⁺⁺) ions. Chelating effect was determined according to the method of Shimada et al. (1992). To 2 ml of the mixture, consisting of 30 mM hexamine (Wako), 30 mM potassium chloride (Sigma) and 9 mM ferrous sulphate (Union Chemical Works, Hsinchu, Taiwan), were added 2 ml of each mycelial extract (0.5-10 mg/ml) in methanol and 200 μl of 1 mM tetramethyl murexide (TMM, Sigma). After 3 min at room temperature, the absorbance of the mixture was determined at 485 nm against a blank. A lower absorbance indicates a higher chelating power. The antioxidant activity in the samples were expressed as Trolox equivalents, using a calibration curve plotted with different concentration of Trolox.

### Results

DPPH radical scavenging ability. DPPH has been widely used to determine the antioxidant capacity of biological samples (Liu et al., 2019). In the presence of free radical scavenger, the single electron of DPPH is captured, which caused the discoloration of the solution and the decrease of the absorbance. Hot water extracts reacted directly with the DPPH radicals and showed significantly different scavenging activities among tested samples. The scavenging effects of hot water extracts against the DPPH radical were evaluated and the results are displayed in Table 8. The concentrations of extracts that inhibited 50% of radical formation (EC₅₀) were also calculated. These antioxidant activities are mainly caused by polysaccharides and/or phenolic compounds existed in extracts obtained from mushrooms. Different extracts prepared from different mushrooms combination showed different from each other DPPH radical scavenging activities, therefore free radical scavenging properties are species dependent.

The results generally showed that all tested samples/extracts exhibited a significantly high ability to scavenge DPPH radicals. Five different concentrations were used for evaluation of DPPH radical scavenging, 0.625 mg/ml. 1.25 mg/ml, 2.5 mg/ml, 5.0 mg/ml, and 10 mg/ml. Radical scavenging abilities gradually increased in parallel with the increase in concentrations, so the radical scavenging activities were dose dependent. At different concentrations (especially at low concentrations 0.625 mg/ml. 1.25 mg/ml. 2.5 mg/ml) of various extracts radical scavenging activities were different. At high concentrations (5.0 mg/ml, and 10 mg/ml) of the tested samples, DPPH scavenging activities were mostly similar with the exception of Spl-5, Spl-6, Spl-11, and Spl-15 and varied from 37.36±6.46% to 45.47±47% at 5 mg/ml, and 41.04±2.12% to 48.41±1.59% at 10 mg/ml. Results obtained were similar or higher to the results obtained by Asatiani et al., (2007) where hot water extracts from 24 submerged mushroom mycelial biomass were investigated. The concentrations of extracts that inhibited 50% of radical formation (EC50) were considerable low as expected after observation of results at different concentrations of extracts used (Table 8). The results displayed in the present invention are most likely due to the high content of polysaccharides, phenolic compounds, and flavonoids existing in the tested samples/extracts. Those results are in agreement with data presented in various studies which show that DPPH scavenging activities in the water or organic solvent extracts obtained from different mushroom species are significantly high in accordance with the high content of phenolic compounds (Liu et al., 2019, Wei et al., 2019). Therefore, the vast majority of tested extracts can be considered as candidates with free radical scavenging properties to be used for production of dietary supplements and/or pharmaceutical products.

TABLE 8
DPPH Scavenging ability of medicinal mushroom extracts

| | Scavenging ability (%) | | | | | |
|---|---|---|---|---|---|---|
| Sample | 0.625 mg/ml | 1.25 mg/ml | 2.5 mg/ml | 5 mg/ml | 10 mg/ml | EC₅₀ |
| Spl-1 | 24.72 ± 2.78 | 36.42 ± 5.84 | 39.67 ± 1.04 | 45.47 ± 3.12 | 47.35 ± 3.37 | 11.15 ± 1.51 |
| Spl-2 | 29.47 ± 3.20 | 35.43 ± 1.49 | 38.29 ± 3.23 | 45.16 ± 0.74 | 48.41 ± 1.59 | 10.76 ± 0.78 |
| Spl-3 | 27.24 ± 2.14 | 36.75 ± 5.67 | 36.79 ± 1.52 | 43.51 ± 2.12 | 45.63 ± 1.25 | 12.25 ± 0.67 |
| Spl-4 | 16.07 ± 8.80 | 27.27 ± 4.97 | 31.92 ± 3.74 | 37.36 ± 6.46 | 41.04 ± 2.12 | 13.6 ± 1.32 |
| Spl-5 | 11.87 ± 0.16 | 18.14 ± 0.59 | 21.88 ± 1.28 | 27.22 ± 0.87 | 31.60 ± 0.16 | 18.84 ± 1.20 |
| Spl-6 | 12.20 ± 2.54 | 19.02 ± 0.60 | 26.19 ± 2.72 | 29.5 ± 2.79 | 37.16 ± 0.27 | 14.87 ± 1.66 |
| Spl-7 | 25.18 ± 2.06 | 31.58 ± 3.62 | 35.71 ± 3.96 | 39.58 ± 2.16 | 45.89 ± 1.77 | 11.87 ± 0.84 |
| Spl-8 | 26.61 ± 1.10 | 34.21 ± 1.94 | 36.24 ± 1.44 | 41.70 ± 2.41 | 42.45 ± 1.11 | 14.51 ± 0.91 |
| Spl-9 | 25.17 ± 3.26 | 32.69 ± 0.80 | 39.38 ± 2.08 | 41.29 ± 1.80 | 41.52 ± 2.56 | 14.83 ± 1.31 |
| Spl-10 | 26.49 ± 1.62 | 36.40 ± 1.69 | 39.00 ± 1.86 | 41.09 ± 4.75 | 43.97 ± 1.68 | 13.29 ± 1.11 |
| Spl-11 | 10.7 ± 1.97 | 21.11 ± 1.21 | 26.72 ± 1.78 | 30.19 ± 2.69 | 30.64 ± 1.35 | 19.18 ± 1.32 |
| Spl-12 | 27.17 ± 0.16 | 36.90 ± 2.42 | 42.35 ± 1.35 | 43.46 ± 2.18 | 45.30 ± 1.99 | 12.54 ± 1.38 |
| Spl-13 | 20.79 ± 0.78 | 31.41 ± 2.63 | 36.55 ± 0.88 | 40.11 ± 1.27 | 42.45 ± 1.56 | 13.34 ± 1.07 |
| Spl-14 | 24.09 ± 1.72 | 33.84 ± 2.16 | 36.26 ± 0.25 | 41.40 ± 1.87 | 44.29 ± 0.06 | 12.66 ± 0.21 |
| Spl-15 | 23.97 ± 2.81 | 32.91 ± 2.64 | 35.47 ± 1.89 | 35.92 ± 3.06 | 38.85 ± 0.18 | 17.20 ± 1.47 |

ABTS radical scavenging ability. ABTS is considered the best free radical reagent to be used to measure electron/hydrogen donating potential of natural substances such as mushroom extracts. The reaction of antioxidants with ABTS radicals will cause the solution fade and the decrease of characteristic absorbance, the more obvious of the solution discoloration, the stronger of the total antioxidant ability of the tested substance (Zhao et al., 2008). Thus, the ABTS radical scavenging performance of antioxidative molecule can be determined by monitoring decrease in absorbance. ABTS has been widely used to determine the antioxidant capacity of biological samples and foods (Liu et al., 2019; Kim et al., 2020b). The percent inhibition of ABTS radicals of studied mushroom extract measured significant results at different concentrations and results are presented in Table 9. Extracts showed the extremely high antioxidant activity determined by ABTS tests, in addition to having the high concentration and variety of phenolic compounds, and flavonoids identified. The ABTS scavenging activity at highest concentration (10 mg/ml) in all tested samples were similar and almost equal to the 100% (varied in 93.84±1.61%-99.88±88% range). At the low concentrations scavenging activities gradually decreased but still maintained sensible activities even at the concentration 1.25 mg/ml. In all tested samples, average scavenging activities at the concentration 5 mg/ml were ˜70%, which is one of the high rates and indicates that the extracts have high activities even at relatively low concentrations. At the concentration 2.5 mg/ml all ABTS scavenging activities were similar ≈40%. The concentrations of extracts that inhibited 50% of ABTS radicals (EC50) were significantly low (3.10±0.04%-3.96±0.01%) as expected considering high activities at different concentrations of extracts used (Table 9). Scavenging of ABTS radicals at such a low EC₅₀ values was being highly appreciated by earlier reports (Knezevic et al. 2018, Wei et al., 2019).

As a conclusion, a vast majority of tested extracts demonstrate extremely high scavenging abilities of ABTS radicals unlike of DPPH scavenging properties. This indicates that these extracts possess exceptionally high antioxidant activities and properties that are highly beneficial and promising and successfully can be used as a natural mycotherapeutic antioxidants for the prevention and treatment of oxidative damages of human body.

TABLE 9
ABTS radicals scavenging ability of medicinal mushroom extracts

| | Scavenging ability (%) | | | | | |
|---|---|---|---|---|---|---|
| Sample | 0.625 mg/ml | 1.25 mg/ml | 2.5 mg/ml | 5 mg/ml | 10 mg/ml | EC₅₀ |
| Spl-1 | 11.38 ± 0.41 | 20.22 ± 0.75 | 40.52 ± 6.60 | 63.64 ± 0.16 | 93.84 ± 1.61 | 3.42 ± 0.54 |
| Spl-2 | 12.00 ± 0.13 | 21.79 ± 0.72 | 40.66 ± 0.83 | 68.59 ± 1.38 | 98.84 ± 0.17 | 3.34 ± 0.03 |
| Spl-3 | 11.82 ± 0.44 | 22.30 ± 0.56 | 40.44 ± 0.40 | 68.80 ± 0.19 | 99.45 ± 0.11 | 3.34 ± 0.03 |
| Spl-4 | 11.07 ± 0.41 | 19.62 ± 0.41 | 35.18 ± 0.52 | 60.50 ± 0.25 | 96.89 ± 0.06 | 3.96 ± 0.01 |
| Spl-5 | 11.35 ± 0.55 | 19.87 ± 0.28 | 37.15 ± 0.52 | 64.76 ± 0.56 | 97.16 ± 0.65 | 3.66 ± 0.03 |
| Spl-6 | 11.55 ± 0.28 | 20.99 ± 0.67 | 38.30 ± 0.76 | 62.89 ± 0.58 | 99.26 ± 0.13 | 3.69 ± 0.06 |
| Spl-7 | 12.77 ± 0.65 | 22.23 ± 0.11 | 40.54 ± 1.15 | 69.18 ± 1.13 | 99.36 ± 0.18 | 3.33 ± 0.10 |
| Spl-8 | 12.37 ± 0.23 | 22.95 ± 0.41 | 42.71 ± 0.95 | 69.95 ± 1.98 | 99.62 ± 0.13 | 3.17 ± 0.12 |
| Spl-9 | 11.58 ± 0.12 | 21.69 ± 0.84 | 40.09 ± 0.77 | 69.56 ± 3.24 | 99.15 ± 0.29 | 3.35 ± 0.05 |
| Spl-10 | 12.72 ± 0.14 | 22.97 ± 0.23 | 42.19 ± 0.78 | 72.88 ± 0.89 | 99.88 ± 0.00 | 3.14 ± 0.06 |
| Spl-11 | 12.48 ± 0.54 | 23.01 ± 0.52 | 41.54 ± 0.48 | 70.73 ± 0.69 | 99.60 ± 0.07 | 3.22 ± 0.02 |
| Spl-12 | 12.54 ± 0.30 | 23.53 ± 0.21 | 42.07 ± 0.48 | 70.36 ± 0.91 | 99.48 ± 0.28 | 3.20 ± 0.05 |
| Spl-13 | 11.49 ± 0.35 | 21.33 ± 0.25 | 39.44 ± 0.14 | 66.25 ± 0.63 | 97.46 ± 0.32 | 3.49 ± 0.03 |
| Spl-14 | 12.51 ± 0.37 | 23.11 ± 1.06 | 42.97 ± 0.49 | 72.14 ± 0.58 | 99.92 ± 0.14 | 3.10 ± 0.04 |
| Spl-15 | 11.15 ± 1.19 | 21.89 ± 0.44 | 39.48 ± 0.49 | 66.09 ± 0.50 | 98.73 ± 0.19 | 3.49 ± 0.05 |

Chelating effects on ferrous (Fe⁺⁺) ions. Antioxidants can remove free radicals directly by providing protons and/or electrons, or indirectly by inhibiting the activity of endogenous oxidases, enhancing the activity of antioxidant enzymes, and chelating metal ions involved in the production of radicals. The Fe²⁺ binding capacity of mushroom extracts was tested at concentrations range of 0.625-10 mg/mL. The results obtained are depicted in Table 10. All examined samples/extracts exhibited significant chelating capacities, higher for the ample Spl-13 the extracts of which at concentrations of 10 mg/ml reached the 90.71±2.10% chelating activity and comparatively lower for samples Spl-6, Spl-8, and Spl-14 of which at concentrations of 10 mg/ml reached the 72.99±3.18%, 67.25±1.80%, and 72.73±4.29%, respectively. Chelating abilities of extracts investigated in the frame of present invention were comparable to previously reported data (Zhou et al., 2020). This finding is in contrast to the higher total phenolics and flavonoid content, as well as the higher reducing power and ABTS radical scavenging activity observed for the tested samples. Generally, all tested extracts demonstrate extremely high chelating abilities on ferrous (Fe⁺⁺) ions at the concentrations 5 mg/ml and 10 mg/ml, but in contrary to this at the concentration 2.5 mg/ml chelating abilities were almost 2-5-fold lower to compare to the 5 mg/ml esed. At lowest concentration (0.625 mg/ml) chelating effects of extracts were in trace amount and none of them exceeded 5.6%. The samples Spl-3, Spl-5, and Spl-6 showed the lowest activities (<1%) at this concentration. Moreover, the results of Fe²⁺ binding capacity of extracts herein were normalized and expressed as EC₅₀ values for comparison. In this study, EC₅₀ values mean the corresponding extract concentrations when the ferrous ion chelating ability reach 50%. Generaly in all tested samples EC₅₀ values were considerable low (<5 mg/ml). Only for Spl-14 and Spl-15 EC₅₀ values were slightly gigher and reached 6.61±1.56 mg, and 7.12±0.07 mg, respectively (Table 10). Generally, the antioxidant properties are inversely correlated with their EC₅₀ values. It was reported that, very strong and significant correlations were found between EC₅₀ value of the chelating ability and total polysaccharides and total protein contents from. The influence of the total protein content may have originated from the remnants of mushroom tyrosinase that could have catalyzed ferous ions oxidation and have led to increase of chelation.

Trolox equivalent antioxidant capacity of mushroom extracts. The trolox equivalent antioxidant capacity assay was developed as a simple and convenient method for total antioxidant capacity determination.

TABLE 10
Chelating effects on ferrous (Fe++) ions

| | Chelating ability (%) | | | | | |
|---|---|---|---|---|---|---|
| Sample | 0.625 mg/ml | 1.25 mg/ml | 2.5 mg/ml | 5 mg/ml | 10 mg/ml | EC₅₀ |
| Spl-1 | 3.61 ± 0.02 | 11.6 ± 0.56 | 15.59 ± 1.62 | 35.93 ± 2.65 | 81.75 ± 0.68 | 5.47 ± 0.93 |
| Spl-2 | 1.17 ± 0.40 | 10.50 ± 1.05 | 26.15 ± 1.92 | 66.65 ± 2.33 | 74.17 ± 2.58 | 3.83 ± 0.91 |
| Spl-3 | 0.35 ± 0.74 | 0.67 ± 0.06 | 17.53 ± 0.89 | 59.53 ± 3.14 | 80.38 ± 1.25 | 4.44 ± 0.14 |
| Spl-4 | 2.42 ± 0.44 | 4.37 ± 0.53 | 32.47 ± 2.09 | 71.97 ± 5.39 | 85.50 ± 1.81 | 3.62 ± 0.19 |
| Spl-5 | 0.52 ± 0.61 | 13.39 ± 0.99 | 53.53 ± 4.36 | 83.43 ± 5.69 | 86.10 ± 3.48 | 2.42 ± 0.17 |
| Spl-6 | 0.11 ± 0.02 | 2.50 ± 0.53 | 16.56 ± 0.39 | 45.86 ± 3.53 | 72.99 ± 3.18 | 5.70 ± 0.39 |
| Spl-7 | 5.59 ± 0.69 | 13.78 ± 1.73 | 31.34 ± 1.20 | 72.15 ± 0.46 | 84.53 ± 3.41 | 3.64 ± 0.11 |
| Spl-8 | 3.61 ± 0.45 | 7.14 ± 0.61 | 11.35 ± 1.24 | 59.46 ± 2.15 | 67.25 ± 1.80 | 4.52 ± 0.23 |
| Spl-9 | 4.18 ± 0.95 | 7.19 ± 1.40 | 28.25 ± 1.52 | 77.06 ± 0.41 | 82.07 ± 2.87 | 3.61 ± 0.13 |
| Spl-10 | 4.37 ± 0.41 | 7.77 ± 0.81 | 24.74 ± 1.56 | 57.96 ± 1.39 | 78.06 ± 2.21 | 4.39 ± 0.16 |
| Spl-11 | 1.98 ± 0.65 | 6.69 ± 1.09 | 37.87 ± 1.29 | 80.04 ± 4.04 | 82.34 ± 3.98 | 3.21 ± 0.19 |
| Spl-12 | 3.82 ± 0.04 | 6.50 ± 0.74 | 31.20 ± 2.92 | 75.90 ± 1.44 | 76.11 ± 2.78 | 3.51 ± 0.23 |
| Spl-13 | 2.55 ± 2.64 | 13.02 ± 1.37 | 26.22 ± 2.56 | 88.21 ± 1.03 | 90.71 ± 2.10 | 3.46 ± 0.08 |
| Spl-14 | 1.25 ± 0.17 | 5.24 ± 0.58 | 16.35 ± 0.67 | 36.94 ± 10.46 | 72.73 ± 4.29 | 6.61 ± 1.56 |
| Spl-15 | 1.78 ± 2.25 | 2.13 ± 1.83 | 8.78 ± 2.76 | 35.26 ± 1.46 | 69.90 ± 0.87 | 7.12 ± 0.07 |

Trolox is water-soluble analog of vitamin E, which is well known antioxidant compound widely used as a positive control for evaluation of antioxidant activities in different substances. The assay measures the ability of antioxidants to scavenge the stable radical cations such as DPPH, or ABTS, a blue-green chromophore that decreases in its color intensity in the presence of antioxidants (Zhong and Shahidi, 2015). This approach is widely employed to evaluate the antioxidant potential of many biologically active substances obtained from plants and mushrooms.

The antioxidant activities of mycelial hot water extracts were evaluated by means of Trolox equivalent (TE) antioxidant capacity in % and results are presented in Table 11. DPPH radical scavenging activities in all tested samples were surprisingly low and were less than 1% (TE mg/g). Lowest DPPH radical scavenging values were observed 0.26±0.01%, 0.27±0.07%, and 0.24±0.06% (TE g/mg) in samples Spl-5, Spl-6, and Spl-11, respectively. Samples Spl-5 and Spl-6 were prepared from the same combinations of mushroom species (P. ostreatus, G. lucidum, A. auricula-judae, T. versicolor, and L. edodes) but in slightly different proportions. For preparation of Spl-11 were used only two mushroom species I. obliquus and G. lucidum. In comparison, ABTS scavenging activities were significantly high. Spl-7-Spl-15 demonstrate similar activities ranged 71.02±0.43%-75.87±0.50% (TE mg/g). In contrast, Spl-1-Spl-6 shoved slightly lower activities and ranged 62.00±0.24%-69.94±0.18% (TE mg/g). Those results are similar or significantly higher to the data presented (Zhy et al., 2019, Alkan et al., 2020). High scavenging properties of tested extracts are most likely caused by considerable high contents of water-soluble polysaccharides, phenolic compounds and flavonoids in the samples.

Beside this, reducing power was evaluated in all 15 samples. Ferric reducing antioxidant power assay is a widely used, simple, sensitive, precise, and inexpensive method that uses antioxidants as reductants in a redox-linked colorimetric reaction. In the reducing power assay, antioxidants convert ferric (Fe³⁺) to ferrous (Fe²⁺) ions via donation of an electron and the resulting ferrous ion (Fe²⁺) formation can be measured spectrophotometrically at 700 nm. This donation of electrons to reactive free radical species, promotes termination of free radical chain reactions. Consequently, the iron colorimetric probe complex develops a dark blue colour. Results of reducing power of tested extracts are presented in Table 11. Similar to ABTS radical scavenging activities, examined extracts depicted the strongest reductive abilities in these assays. However, it should be noted that no dose-dependence relationship could be observed among samples. It is reported that the reducing power of mushrooms based on their phenolics and flavonoids contents (Froufe et al. 2011). According to this fact it was expected the high values of reducing power of tested extracts, which ranged within 52.72%-65.00-% (TE mg/g) and were similar to each other.

Moreover, chelating of ferrous ions was estimated in all samples. Chelation of metal ions is often associated with redox active metal catalysis, which prevents generation of ROS. Bioactive substances produced by mushrooms are able to chelate metals or to delocalize the electronic charge coming from free radicals. In this assay, chelating agents disrupt the ferrozine-Fe²⁺ complex, thus decreasing the red color intensity. Measurement of the rate of color reduction at 485 nm, therefore, allows estimation of the chelation activity. A lower absorbance value indicates a higher chelation ability. All tested extracts exhibited significant chelating capacities, higher for the Spl-5, Spl-7, Spl-11, and Spl-13 samples the extracts of which reached the 34.28±2.33%. 28.90±0.27%, 30.15±2.19%, and 34.40±0.55%, respectively of TE mg/g chelating activity and significantly lower for Spl-9, Spl-10, Spl-14, and Spl-15 demonstrate 6.25±0.43%, 16.40±1.05%, 15.58±4.66%, and 14.88±0.24% TE mg/g chelating activity respectively (Table 11). It is reported that, high chelating ability against ferrous ion, indicating that the chelating effect might impart polysaccharides capable of antioxidant potentials (Li et al., 2011), which was expected due to the significant content of polysaccharides in the extracts. Moreover, chelating effect might partly be due to the presence of functional groups such as carboxyl group and sulfuric radical in the polysaccharide structure (Wang et al. 2016).

TABLE 11
Antioxidant properties of medicinal mushroom
extracts in trolox equivalent (trolox mg/g)

| Sample | DPPH | ABTS | Reducing power | Chelating Fe⁺⁺ ions |
|---|---|---|---|---|
| Spl-1 | 0.90 ± 0.05^{A} | 64.29 ± 0.16^{H} | 56.28 ± 0.42^{F} | 18.23 ± 4.66^{EFG} |
| Spl-2 | 0.82 ± 0.04^{AB} | 69.45 ± 1.34^{F} | 58.38 ± 0.39^{CD} | 20.43 ± 1.22^{DE} |
| Spl-3 | 0.63 ± 0.02^{C} | 69.94 ± 0.18^{EF} | 56.59 ± 0.80^{FF} | 21.78 ± 1.24^{DE} |
| Spl-4 | 0.86 ± 0.03^{A} | 62.00 ± 0.24^{I} | 52.72 ± 0.83^{H} | 24.54 ± 3.51^{CD} |
| Spl-5 | 0.26 ± 0.01^{D} | 66.39 ± 0.53^{G} | 54.07 ± 0.62^{G} | 34.28 ± 2.33^{CD} |
| Spl-6 | 0.27 ± 0.07^{D} | 65.09 ± 0.55^{GH} | 57.18 ± 0.98^{EF} | 19.08 ± 1.46^{EFG} |
| Spl-7 | 0.65 ± 0.02^{C} | 71.55 ± 1.05^{DE} | 59.51 ± 0.65^{C} | 28.90 ± 0.27^{BC} |
| Spl-8 | 0.65 ± 0.08^{C} | 72.36 ± 1.84^{CD} | 57.74 ± 0.69^{DE} | 19.68 ± 3.07^{EFG} |
| Spl-9 | 0.77 ± 0.02^{B} | 72.46 ± 2.93^{CD} | 55.13 ± 0.86^{G} | 6.25 ± 0.43^{BC} |
| Spl-10 | 0.63 ± 0.09^{C} | 75.73 ± 0.79^{A} | 61.41 ± 0.80^{B} | 16.40 ± 1.05^{FGH} |
| Spl-11 | 0.24 ± 0.06^{D} | 74.43 ± 0.61^{AB} | 65.00 ± 0.97^{A} | 30.15 ± 2.19^{B} |
| Spl-12 | 0.77 ± 0.03^{B} | 73.80 ± 0.80^{BC} | 56.74 ± 0.31^{EF} | 27.99 ± 2.77^{BC} |
| Spl-13 | 0.89 ± 0.03^{A} | 70.57 ± 0.55^{DE} | 61.20 ± 0.56^{B} | 34.40 ± 0.55^{A} |
| Spl-14 | 0.66 ± 0.04^{C} | 75.87 ± 0.50^{A} | 58.97 ± 0.25^{C} | 15.58 ± 4.66^{GH} |
| Spl-15 | 0.73 ± 0.07^{C} | 71.02 ± 0.43^{DE} | 56.46 ± 0.51^{F} | 14.88 ± 0.24^{H} |

Statistical Analyses. Each measurement and analyses were conducted in triplicate. The data from 3 replications were analyzed using SAS soft-ware (SAS Institute, Inc., Cary, NC). The Duncan multiple range test was used to determine the differences of each group, and a P value <0.05 was considered to be statistically significant. The results were expressed as mean±standard error (n=3). Means with different letters within a column differ significantly (p<0.05).

### Example 10. The Effect of “Immunity Formula” on LPS-Induced Macrophages as an In Vitro Model of Inflammation Using RAW264.7 Macrophages

Macrophages play important roles in the host-immune system during infection, and their activation may lead to various chronic inflammatory diseases such as rheumatoid arthritis, tuberculosis, asthma, sinusitis, Crohn's disease, ulcerative colitis, active hepatitis, and viral infections. Macrophages can be activated by different stimuli, including lipopolysaccharide (LPS, an endotoxin from gram-negative bacteria), leading to the release of pro-inflammatory cytokines such as tumor necrosis factor-alpha (TNF-α), interleukin (IL)-1β and IL-6, inflammatory mediators like nitric oxide (NO), prostaglandin (PG) E2, and production of ROS, which significantly contribute to the induction of peripheral inflammatory diseases as well as neuro-inflammatory diseases. The synthesis of PGs from arachidonic acid is catalyzed by cyclooxygenase (COX). One of the two isomers of COX, COX-2, is highly inducible by inflammatory stimuli, such as an endotoxin LPS, and the targeted inhibition of COX-2 could thus be a promising approach to prevent and suppress inflammatory response.

Medicinal mushrooms are known as capable of activating an immunosuppressed system via stimulation of innate and adaptive immunity, by the binding of specific ligands to the pathogen recognition receptors (PRRs) which initiate both innate and adaptive immunological responses to specific pathogens such as fungi, bacteria and yeast. Binding to membrane-bound PRRs that include Toll like receptors (TLRs), C-type lectin receptors (CLRs), dectin-1 and complement receptor 3 (CR3) by pathogen-associated molecular patterns such as polysaccharides can activate immune responses by enhancing the secretion of inflammatory cytokines such as TNF-α and IL-6. PRRs also serve to bind ligands that prime immune responses. Mushroom extracts have thus been used for immunomodulation.

In the present study, the anti-inflammatory effect of a particular mushroom-cannabidiol extract (referred to hereinafter as “Immunity formula”) was assessed in an in vitro model of inflammation using RAW264.7 murine macrophage cell line stimulated with LPS. The hypothesis underlying the study was that the Immunity formula can serve as a potent inhibitor of over-production of inflammatory mediators by macrophages exposed to LPS and may therefore be a good candidate for the prevention and suppression of inflammatory responses in the periphery and brain.

### Materials and Methods

RAW264.7 murine macrophage cells. RAW264.7 macrophage cells were cultured at 37° C. with 5% CO₂ and 90% relative humidity. Cells were incubated in Dulbecco's modified Eagle's medium (DMEM) containing 4.5 g/L glucose and 2 mM L-glutamine and supplemented with 10% fetal bovine serum, 1% sodium pyruvate, penicillin (100 U/mL), and streptomycin (100 μg/mL).

LPS exposure. RAW264.7 cells were seeded in a 12-wells plate (10⁵ cells/well) in serum containing medium and grown for 24 hours. The cells were exposed to LPS (20 ng/ml) for the next 24 hours.

Immunity formula and its treatment. The Immunity formula tested in the present study was a combination of five hot water extracts (in equal parts by volume), each of a different one of the Basidiomycetes mushrooms Trametes versicolor CBS 147000, Cordyceps militaris (L.) Link, Flammulina velutipes CBS 146995, Ganoderma lucidum CBS 146996, and Lentinus edodes (Berk.) Singer; and an ethanol (70%) extract of said Ganoderma lucidum species, in a ratio of 9:1, respectively, by volume, and was enriched by isolated hemp CBD (1% by volume of the overall mixture) (all extracts were purchased from Shanghai Sanmin Herbal Industrial Co., Ltd, China). The water extracts used are highly rich in polysaccharides; and the reason for mixing them with an ethanol extract of the Ganoderma lucidum was the amount of triterpens found in an alcoholic extract of said mushroom specis, which is remarkeably higher than that found in a water extract of the same species. Extracts were dissolved and combined to form a homogenous mix, and stored at 4° C. RAW264.7 cells were treated with the Immunity formula (20 μg/ml) together with LPS exposure. The cell culture supernatant was collected, aliquoted, and stored at −20° C. until used.

Trypan blue staining for cell counting. The cells were scrapped off before seeding into a 12-wells plate. This procedure includes 1:1 ratio of cells and trypan blue dye in an Eppendorf tube. Ten microliter of this mixture was loaded into the Neubauer's slide covered with a cover slip. The cells were seeded in a 12-wells plate (10⁵ cells/well) in complete medium.

Enzyme linked immunosorbent assay (ELISA). Sample preparation: Cell culture supernatants were carefully collected from 12-wells plate, aliquoted, and stored at −20° C. until assayed.

Plate preparation and standards for IL-1β (ab197742), IL-6 (ab222503), TNF-α (ab208348), and COX-2 (ab210574): The ELISA kits were provided as ready to use plates and wells (Abcam). Extra wells were removed from the plate and stored in 4° C. in dark. Standards were serially diluted according to manufacturer protocol and the samples were defrosted at room temperature. Antibody cocktail (capture and detection antibody) was diluted in antibody diluent according to the manufacturer's instructions. Total of 50 μL of standards, all samples, and then prepared antibody were added to the appropriate wells. The wells were sealed and incubated for 1 hour at room temperature on a plate shaker at 100 rpm. Each plate was washed with 4×300 μL 1× wash buffer PT (Abcam). 3,3′,5,5′-Tetramethylbenzidine (TMB) substrate (100 μL) was added, and samples were incubated for 5-15 minutes in the dark on a plate shaker (depending upon the appearance of color). Total of 100 μL of stop solution was added and mixed on a shaker for 1 minute to stop the reaction. Optical density (OD) was recorded at 450 nm with endpoint reading.

For CCL5 (ab213736), NUNC 96-well plate was coated with 50 μl of 2 μg/ml capture antibody (diluted in coating buffer) for 2 hours at room temperature on shaker (100 rpm). Then the plate was washed with 4×300 μL 1× wash buffer and blocked with 300 μl blocking buffer at 4° C. on shaker overnight. Next day, the plate was washed again with 4×300 μL 1× wash buffer and 50 μl of standard and sample was used. The plate was again incubated for 2 hours at room temperature on shaker. The washing process was repeated and 50 μl of 0.5 μg/ml of detection antibody was incubated for 2 hours at room temperature on shaker. The plate was washed after 2 hours, and streptavidin 0.05 μg/ml was added (50 μl in each well) and incubated at room temperature for 1 hour on shaker. The plate was washed as it is described before and 100 μl of TMB substrate was used in the plate and incubate for 5-15 minutes until colorization was appeared. Stop solution was used to stop the reaction, and OD was recorded at 450 nm with endpoint reading.

Nitrite assay. RAW264.7 cells were seeded in 12-wells plate and incubated for 24 hours in complete medium. Then, the cells were exposed to LPS (20 ng/ml) and treated with the Immunity formula (20 μg/ml). Nitrite production was determined by measuring the levels of the NO metabolite nitrite (NO₂) in the medium by using a colorimetric reaction with Griess reagent. Then 100 μl of cell culture supernatant and Griess reagent were mixed (1:1). Sodium nitrate, 0.1 M was used to build a calibration curve. Absorbance was measured at 540 nm after 15 min on a shaker (50 rpm), with a spectrophotometer Zenyth 200 (Anthos, Eugendorf, Austria).

LDH assay. When plasma membranes of the cell damages, cytoplasmic enzyme LDH releases and which also indicates necrosis. Cytotoxicity was analyzed using lactate dehydrogenase cytotoxicity assay kit (ab102506, Abcam, USA). The cells were seeded in 96-wells plate (10,000 cells/well) and incubated for 24 hours. The LPS exposure and Immunity treatment was given to the cells in starvation medium. Then, 100 μl of cell culture supernatant was transferred into new plate. The reaction mixture from the kit was added to the sample in new plate (1:1) and incubated for 15 minutes on shaker at 50 rpm. The amount of formazan formed is proportional to the amount of LDH release. The color intensity is proportional to the number of damaged cells. The OD was read at 492 nm with reference wavelength of 690 nm at endpoint reading with a spectrophotometer Zenyth 200.

### Results

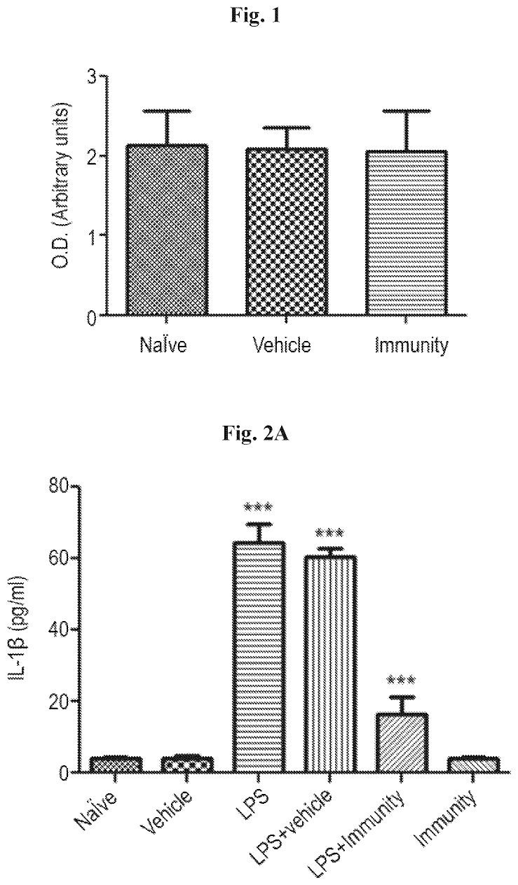

Cytotoxicity analysis with LDH assay. In order to check a possible cytotoxic effect of the Immunity formula, the LDH release from RAW264.7 macrophages exposed to the Immunity formula was measured. The LDH release was compared between cells exposed to the tested formula, cells exposed to a corresponding vehicle, and naïve cells. As shown in FIG. 1, no toxic effects were detected in the three cell groups.

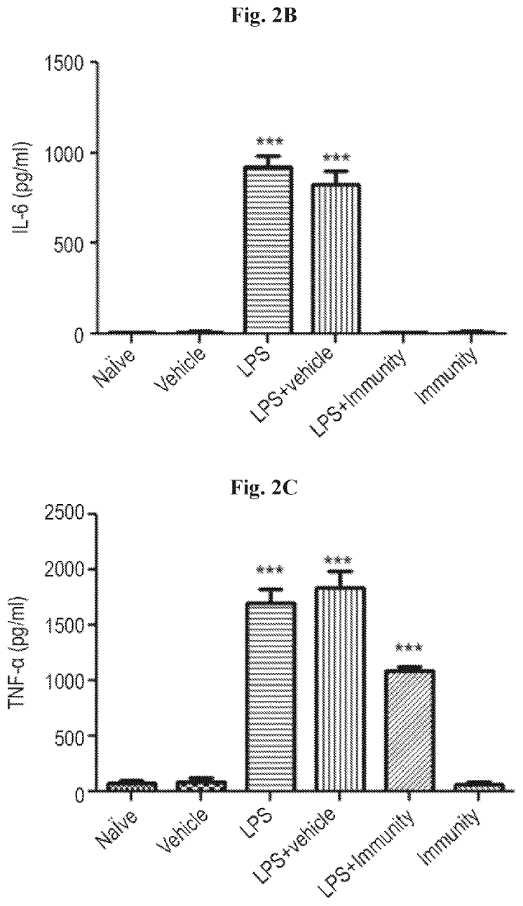

Pro-inflammatory cytokines. As shown in FIG. 2, LPS induced a dramatic increase of the inflammatory mediators IL-1β (A), IL-6 (B), and TNF-α (C), while vehicle did not significantly affect the levels of these mediators. LPS+ vehicle did not affect the release of the inflammatory mediators. The Immunity formula significantly counteracted the release of the pro-inflammatory cytokines. The release of IL-6 was inhibited completely (FIG. 2B); the release of IL-1B was inhibited by 3.9-folds as compares to LPS group (FIG. 2A) whereas TNF-α was inhibited by 1.5-folds (FIG. 2C). The Immunity formula did not show any effect on its own.

Chemokine. The effect of the Immunity formula was measured on CCL5 (RANTES) release, as a part of pro-inflammatory response using ELISA. Naïve and vehicle did not affect CCL5 release, while CCL5 levels were increased in the LPS group as compared to vehicle. LPS+ vehicle did not show any significant effect as compared to LPS itself, whereas in LPS+ Immunity group, the release of CCL5 was inhibited completely and remain in the naïve/vehicle range (non-significant). The Immunity formula alone did not affect CCL5 levels (FIG. 3).

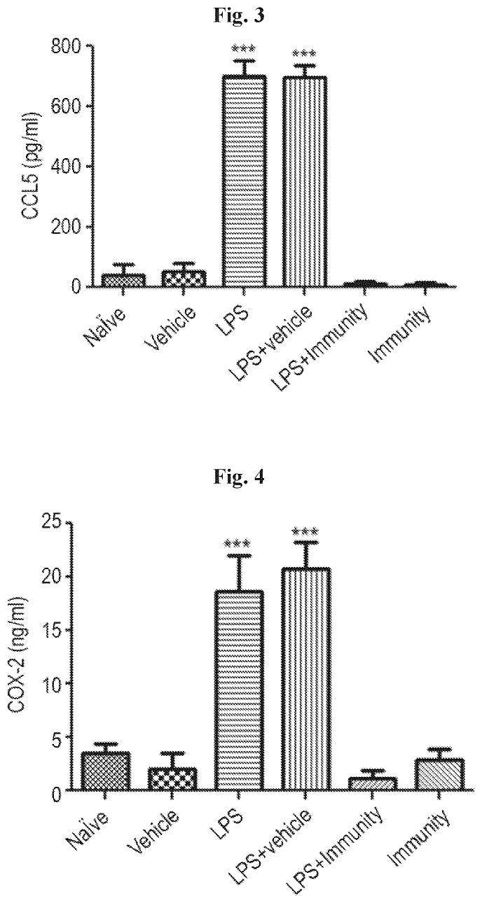

Intracellular inflammatory marker-COX-2. COX-2 levels were measured by ELISA using cells lysate samples as a part of intracellular inflammatory response. LPS increased the COX-2 protein expression up to 5.3-folds compared to naïve/vehicle groups and LPS+ vehicle have also shown similar effect. In contrast, in LPS+ Immunity group, the release of COX-2 was suppressed almost completely (insignificant compared to the naïve/vehicle groups). The Immunity formula alone did not affect COX-2 release (FIG. 4).

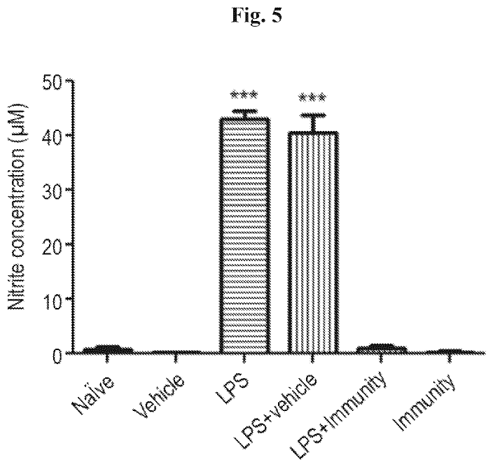

Nitrite assay. Nitrite levels in the RAW264.7 macrophage cell culture supernatant was assessed by Griess reaction. The nitrite levels were increased up to 56.9-folds in comparison to naïve/vehicle groups. LPS+ vehicle did not differ significantly from LPS alone. The Immunity formula (20 μg/ml) counteracted this dramatic increase and suppressed the release to the naïve/vehicle levels range. The Immunity formula alone did not differ from the control groups, i.e., has no effect by itself (FIG. 5).

### DISCUSSION

In order to assess the effect of the Immunity formula on LPS-induced RAW264.7 cells, several inflammatory markers including IL-1β, TNF-α, IL-6, CCL5, COX-2, NO, and LDH (as a marker for necrosis) were analyzed. As hypothesized, the formula tested was able to completely prevent the LPS-induced release of IL-6 and partially prevent the release of IL-1β and TNF-α (FIG. 2). TNF-α is a potent endotoxin which activates inflammatory pathways and can cause several chronic inflammatory diseases. Excessive production of TNF-α by macrophages can also lead to serious pathological conditions such as septic shock and rheumatoid arthritis. IL-1β is a cytokine which belongs to IL-1 peptide group, and is suggested to play a role in tumorigenesis and also inflammatory activation.

NO is a very unstable and harmful molecule which is the product of arginine metabolism and released during the activation of M1 inflammatory pathway. This unstable molecule is converted into a stable molecule nitrite/nitrate. The Immunity formula inhibited NO production in LPS-stimulated RAW264.7 cells to control range, indicating complete inactivation of reactive nitrogen species production, and showed complete inhibition of COX-2 elevation as well in LPS-induced RAW264.7 cells. Prostaglandins are lipid molecules formed from arachidonic acid, which mediate pathogenic mechanisms and are important markers of inflammatory response. The ability to inhibit COX-2 expression makes the Immunity formula applicable for treatment of chronic inflammatory diseases such as gouty arthritis.

CCL5 is a chemokine capable of binding to CCR5 receptor and other chemokine receptors, which is known as an important marker in inflammation. Overexpression of CCL5 may leads to pathological and inflammatory states. The Immunity formula demonstrated complete inhibition of CCL5 production in LPS stimulated RAW264.7 cells showing that said formula has immunomodulatory effects as reflected by the suppression of CCL5 expression in macrophages, which is responsible for recruiting leukocytes at a site of inflammation.

In order to assess whether the Immunity formula has in vitro toxic effects, LDH assay was performed, and the results approved the safety of the tested formula (FIG. 1) at least with regard to RAW264.7 cells.

In conclusion, the present study shows that the Immunity formula possesses anti-inflammatory and immunomodulatory effects which inhibit not only the release of pro-inflammatory cytokines, but also the release of relevant chemokine (CCL5), prostaglandins (COX-2), and nitrite release. This potent and broad anti-inflammatory activity may be relevant to several chronic inflammatory diseases. Although the tested formula did not display a cytotoxic effect on RAW274.7 cells, as reflected by the lack of LDH elevation, studies with more immune cells, as well as further investigation in in vivo models of inflammation, are required.

### REFERENCES

AACC International. 2000. Approved methods of the AACC (10th Ed.). St. Paul, MN, USA: American Association of Cereal Chemists.
Adaskaveg J E, Gilbertson R L. 1986. Cultural studies and genetics of sexuality of Ganoderma Lucidum and G. tsugae in relation to the taxonomy of the G. Lucidum complex. Mycologia. 78: 694-705.
Akata I, Zengin G, Picot C M N, Mahomoodally M F. 2019. Enzyme inhibitory and antioxidant properties of six mushroom species from the Agaricaceae family. S Afr J Bot. 120: 95-99.
Alkan S, Uysal A, Kasik G, Vlaisavljevic S, Berežni S, Zengin G. 2020. Chemical characterization, antioxidant, enzyme inhibition and antimutagenic properties of eight mushroom species: a comparative study. J Fungi. 6: 166.
Alonso E N, Ferronato M J, Fermento M E, Gandini N A, Romero A L, Guevara J A, Facchinetti M M, Curino A C. 2018. Antitumoral and antimetastatic activity of Maitake D-Fraction in triple-negative breast cancer cells. Oncotarget. 9: 23396-23412.
Bach F, Helm C V, Bellettini M B, Maciel G M, Windson C, & Haminiuk I. 2017. Edible mushrooms: a potential source of essential amino acids, glucans and minerals. Int J Food Sci Technol. 52: 2382-2392.
Binion D E, Burdsall H H, Stephenson S L, Miller O K, Roody W C, Binion L N D, Stephenson S, Roody W, Burdsall H H, Miller O K, Vasilyeva L. 2008. Macrofungi associated with oaks of eastern North America. Morgantown, WV: West Virginia University Press. 467 pp.
Borodina I, Kenny L C, McCarthy C M, Paramasivan K, Pretorius E, Roberts T J, Hoek S A, Kell D B. 2020. The biology of ergothioneine, an antioxidant nutraceutical. Nutr Res Rev. 33: 190-217.
Buchalo A S, Wasser S P, Reshetnikov S V, Grigansky A P. 1999. Studies on microstructures of vegetative mycelium in the medicinal mushrooms Hericium erinaceus (Bull.: Fr.) Pers. and Grifola frondosa (Dicks.: Fr.) S. F. Gray (Aphyllophoromycetidae). Int J Med Mushrooms. 1(3): 235-242.
Buchalo A S, Wasser S P, Mykhailova O B, Bilan V T, Lomberg M L. 2011. Taxonomical significance of microstructures in pure cultures of macromycetes. Proceedings of the 7th International Conference on Mushroom Biology and Mushroom Products (ICMBMP7).
Chan J S L, Asatiano M D, Sharvit L, Trabelcy B, Barseghyan G S, Wasser S P. 2015. Chemical composition and medicinal value of the new Ganoderma tsugae var. jannieae CBS-120304 medicinal higher Basidiomycete mushroom. Int J Medicinal Mushrooms. 17: 735-747.
Chang S T, Wasser S P. 2018. Current and future research trends in agricultural and biomedical applications of medicinal mushrooms and mushroom products (review). Int J Med Mushrooms. 20: 1121-33.
Chong P S, Fung M, Wong K H, Lim L W. 2020. Therapeutic potential of Hericium erinaceus for depressive disorder. Int J Mol Sci. 21: 163.
Cohen N, Cohen C, Asatiani M D, Varshney V K, Yu H T, Yang Y C, Li Y H, Mau J L, Wasser S P. 2014. Chemical composition and nutritional and medicinal value of fruit bodies and submerged cultured mycelia of culinary-medicinal higher Basidiomycetes mushrooms. Int J Med Mushrooms. 16: 273-91.
Duan Z, Zhang Y, Zhu C, Wu Y, Du B, Ji H. 2020. Structural characterization of phosphorylated Pleurotus ostreatus polysaccharide and its hepatoprotective effect on carbon tetrachloride-induced liver injury in mice. Int J Biol Macromol. 162: 533-547.
Dubost N J, Beelm R B, Peterson D, Royse D J. 2006. Identification and quantification of ergothioneine in cultivated mushrooms using liquid chromatography mass spectroscopy. Int J Med Mushrooms. 8: 215-222.
Ey J, Scho″mig E, Taubert D. 2007. Dietary sources and antioxidant effects of ergothioneine. J Agric Food Chem. 55: 6466-74.
Fernandes I, Pérez-Gregorio R, Soares S, Mateus N, Freitas V. 2017. Wine flavonoids in health and disease prevention. Molecules. 22: 292.
Froufe H J C, Abreu R M V, Ferreira ICFR. 2011. QCAR models to predict wild mushrooms radical scavenging activity, reducing power and lipid peroxidation inhibition. Chemometr Intel Lab Syst. 109: 192-196.
Garcia J, Afonso A, Fernandes C, Nunes F M, Marques G, Saavedra M J. 2021. Comparative antioxidant and antimicrobial properties of Lentinula edodes Donko and Koshin varieties against priority multidrug-resistant pathogens. South African J Chem Engin. In press.
Golak-Siwulska I, Kałuzewicz A, Spizewski T, Siwulski M, Sobieralski K. 2018. Bioactive compounds and medicinal properties of Oyster mushrooms (Pleurotus sp.). Folia Hortic. 30: 191-201.
Gong S, Zhang H Q, Yin W P, Yin Q Z, Zhang Y, Gu Z L, Qian Z M, Tang P L. 1998. Involvement of interleukin-2 in analgesia produced by Coriolus versicolor polysaccharide peptides. Zhongguo Yao Li Xue Bao. 19: 67-70.
Gonzalez A, Nobre C, Simoes L S, Cruz M, Loredo A, Rodríguez-Jasso R M, Contreras J, Texeira J, Belmares R. 2021. Evaluation of functional and nutritional potential of a protein concentrate from Pleurotus ostreatus mushroom. Food Chem. 346: 128884.
Ha B, Moon Y J, Song Y, Kim S, Kim M, Yoon C W, Ro H S. 2019. Molecular analysis of B mating type diversity in Lentinula edodes. Scientia Horticulturae. 243: 55-63.
Habtemariam S. 2020. Trametes versicolor (Synn. Coriolus versicolor) polysaccharides in cancer therapy: targets and efficacy. Biomed. 8: 135.
Han E S, Oh J Y, Park H J. 2011. Cordyceps militaris extract suppresses dextran sodium sulfate-induced acute colitis in mice and production of inflammatory mediators from macrophages and mast cells. J Ethnopharmacol. 134: 703-710.
He B L, Zheng Q W, Guo L Q, Huang J Y, Yun F, Huang S S. 2020. Structural characterization and immune-enhancing activity of a novel high-molecular-weight polysaccharide from Cordyceps militaris. Int J Biol Macromol. 145: 11-20.
He Y, Li X, Hao C, Zeng P, Zhang M, Liu Y, Chang Y, Zhang L. 2018. Grifola frondosa polysaccharide: a review of antitumor and other biological activity studies in China. Discov Med. 25: 159-176.
Hetland G, Johnson E, Bernardshaw S V, Grinde B. 2020. Can medicinal mushrooms have prophylactic or therapeutic effect against COVID-19 and its pneumonic superinfection and complicating inflammation? Scand J Immunol. e12937.
Horn B, Kay R, Abel D. 1993. A guide to Kansas mushrooms. Kansas University Press. 297 pp.
Hsu C H, Sun H L, Sheu J N, Ku M S, Hu C M, Chan Y. 2008. Effects of the immunomodulatory agent Cordyceps militaris on airway inflammation in a mouse asthma model. Pediatr Neonatol. 49: 171-178.
Huamán-Leandro L R, González-Muñoz M J, Fernández-de-Ana C, Rodríguez-Blanco A, Torres M D, Domínguez H. 2020. Autohydrolysis of Lentinus edodes for obtaining extracts with antiradical properties. Foods. 9: 74.
Jeong H, Yang B, Jeong Y, Kim G, Jeong Y, Kim S, Mehta P, Song C. 2007. Hypolipidemic Effects of Biopolymers Extracted from Culture Broth, Mycelia, and Fruiting Bodies of Auricularia auricula-judae in Dietary-induced Hyperlipidemic Rats. Mycobiology. 35: 16-20.
Jo W, Kim D, Seok S, Jung H, Park S. 2014. The culture conditions for the mycelial growth of Auricularia auricula-judae. J Mushrooms. 12:88-95.
Johnston M V, Adams H P, and Fatemi A 2016 Neurobiology of Disease Oxford University Press, New York.
Jun H I, Song G S, Yang E I, Youn Y, Kim Y S. 2012. Antioxidant activities and phenolic compounds of pigmented rice bran extracts. J Food Sci. 77: 759-764.
Kim S. 2020. Antioxidant Compounds for the inhibition of enzymatic browning by polyphenol oxidases in the fruiting body extract of the edible mushroom Hericium erinaceus. Foods. 9: 951.a
Kim Y, Wu Y, Choi M, Shin H, Li J. 2020. Alginate-dderived elicitors enhance β-glucan content and antioxidant activities in culinary and medicinal mushroom, Sparassis latifolia. J Fungi. 6: 92. B
Knezević A, Stajić M, Sofrenić I, Stanojković T, Milovanović I, Tesević V, Vukojević J. 2018. Antioxidative, antifungal, cytotoxic and antineurodegenerative activity of selected Trametes species from Serbia. PLOS ONE. 13: e0203064.
Kosakyan A, Zmitrovich I V, Didukh M, Wasser S P, Nevo E. 2014. Agaricomycetes of Israel. (P. A. Volz, ed.). Vol. 10 Biodiversity of Cyanoprocaryotes, Algae and Fungi of Israel. Koeltz Scientific Books, Germany, 375 pp.
Lee C, Huang K, Shaw J, Chen J, Kuo W, Shen G, Grumezescu A M, Holban A M, Wang Y, Wang J, Hsiang Y, Lin Y, Hsu H, Yang C. 2020. Trends in the immunomodulatory effects of Cordyceps militaris: total extracts, polysaccharides and cordycepin. Front Pharmacol. 11: 575704.a
Lee H A, Cho J, Afinanisa Q, An G, Han J, Kang H J, Choi S H, Seong H. 2020. Ganoderma lucidum extract reduces insulin resistance by enhancing AMPK activation in high-fat diet-induced obese mice. Nutrients. 12: 3338.b
Lee M, Hur H, Chang K, Lee T, Ka K. Jankovsky L. 2008. Introduction to Distribution and Ecology of Sterile Conks of Inonotus obliquus. Mycobiology. 36: 199-202.
Li H, Xu J, Liu Y. 2011. Antioxidant and moisture-retention activities of the polysaccharide from Nostoc commune. Carbohydr Polym. 83: 1821-1827.
Li X, Chen P, Zhang P, Chang Y, Cui M, Duan J. 2019. Protein-bound beta-glucan from Coriolus versicolor has potential for use against obesity. Mol Nutr Food Res. 63: e1801231.
Li Z, Shi Y, Zhang X, Xu J, Wang H, Zhao L, Wang Y. 2020. Screening immunoactive compounds of Ganoderma lucidum spores by mass spectrometry molecular networking combined with in vivo zebrafish assays. Front Pharmacol. 11: 287.
Lim Y O, Park H W, Kim J H, Lee J Y, Moon S H, Shin C S. 2005. Anti-cancer effect and structural characterization of endo-polysaccharide from cultivate mycelia of Inonotus obliquus. Life Sci. 79:72-80.
Liu M, Yao W, Zhu Y, Liu H, Zhang J, Jia L. 2018. Characterization, antioxidant and antiinflammation of mycelia selenium polysaccharides from Hypsizygus marmoreus SK-03. Carbohydr Polym. 201: 566-574.
Liu X, Hou R, Wang D, MaiM, WuX, ZhengM, Junsheng Fu. 2019. Comprehensive utilization of edible mushroom Auricularia auricula waste residue-extraction, physicochemical properties of melanin and its antioxidant activity. Food Sci Nutr. 7: 3774-3783.
Ma X, Zhou F, Chen Y, Zhang Y, Hou L, Cao X, Wang C. 2014. A polysaccharide from Grifola frondosa relieves insulin resistance of HepG2 cell by Akt-GSK-3 pathway. Glycoconjug J. 31: 355-363.
Masuda Y, Inoue M, Miyata A, Mizuno S, Nanba H. 2009. Maitake ß-glucan enhances therapeutic efect and reduces myelosupression and nephrotoxicity of cisplatin in mice. Int Immunopharmacol. 9: 620-626.
Mau J L, Chyau C C, Li J Y, Tseng Y H. 1997. Flavor compounds in straw mushrooms Volvariella volvacea harvested at different stages of maturity. J Agr Food Chem. 45: 4726-4729.
Moser M. 1983. Die Röhrlinge und Blätterpilze (Polyporales, Boletales, Agaricales, Russulales). Kleine Kryptogameflora IIb/2. 5 Auflage. Gustav Fischer Verlag. Stutgard-New York. 533 S.
Murphy E J, Masterson C, Rezoagli E, O'Toole D, Major I, Stack G D, Lynch M, Laffey J G, Rowan N J. 2020. α-Glucan extracts from the same edible shiitake mushroom Lentinus edodes produce differential in-vitro immunomodulatory and pulmonary cytoprotective effects—Implications for coronavirus disease (COVID-19) immunotherapies. Sci Total Environ. 732:139330.
Ngo D. Sang T Vo. 2019. An Updated review on pharmaceutical properties of gamma-aminobutyric acid. Molecules. 24: 2678.
Nowakowski P. Markiewicz-Zukowska R, Gromkowska-Kępka K, Naliwajko S K, Moskwa J, Bielecka J. Grabia M. Borawska M, Socha K. 2021. Mushrooms as potential therapeutic agents in the treatment of cancer: Evaluation of anti-glioma effects of Coprinus comatus, Cantharellus cibarius, Lycoperdon perlatum and Lactarius deliciosus extracts. Biomed Pharmacother. 133: 111090.
Nuss, P. 2015. Anxiety disorders and GABA neurotransmission: A disturbance of modulation. Neuropsychiatr. Dis Treat. 11: 165-175.
Oyaizu M. 1986. Studies on products of browning reactions: antioxidative activities of products of browning reaction prepared from glucosamine. Japanese J Nutr. 44: 307-315.
Papp V. Bálint D. Wasser S P. 2017. What is Ganoderma lucidum in the molecular era? Int J Med Mushrooms. 19: 575-593.
Pazzi F. Adsuar J C. Domínguez-Muñoz F J, Garcíβ-Gordillo M A, Gusi N, Collado-Mateo D. 2020. Ganoderma lucidum effects on mood and health-related quality of life in women with fibromyalgia. Healthcare. 8: 520.
Pérez-Sánchez A, Barraj'on-Catalán E, Herranz-L'opez M. 2018 Nutraceuticals for skin care: a comprehensive review of human clinical studies. Nutrients. 10: 403-541.
Poniedziałek B, M Siwulski, A Wiater, I Komaniecka, A Komosa, M Gasecka, Z Magdziak, M Mleczek, P Niedzielski, J Proch, M Ropacka-Lesiak, M Lesiak, E Henao, P Rzymski. 2019. The effect of mushroom extracts on human platelet and blood coagulation: In vitro screening of eight edible species. Nutrients. 11: 3040.
Re R. Pellegrini N, Proteggente A. 1999. Antioxidant activity applying an improved ABTS radical cation decolorization assay. Free Radic Biol Med. 26: 1231-7.
Rigling M, Yadav M, Yagishita M, Nedele A, Sun J, Zhang Y. 2021. Biosynthesis of pleasant aroma by enokitake (Flammulina velutipes) with a potential use in a novel tea drink. LWT. 140: 110646.
Ryvarden L, Melo I. 2014. Poroid Fungi of Europe. Synopsis Fungorum. Vol 31. Fungiflora, Norway, 457 pp.
Shahzad F, Anderson D, Najafzadeh M. 2020. The antiviral, anti-Inflammatory effects of natural medicinal herbs and mushrooms and SARS-COV-2 infection. Nutrients. 12: 2573.
Shimada K, Fujikawa K, Yahara K, Nakamura T. 1992. Antioxidative properties of xanthan on the autoxidation of soybean oil in cyclodextrin emulsion. J Agr Food Chem. 40: 945-948.
Singh U, Gautam A, T K Singha, Tiwari A, Tiwari P, Sahai V, Sharma S. 2020. Mass production of Pleurotus eryngii mycelia under submerged culture conditions with improved minerals and vitamin D2. LWT-Food Sci Technol. 131: 109665.
Sohretoglu D, Huang S. 2018. Ganoderma lucidum polysaccharides as an anti-cancer agent. Anticancer Agents Med Chem. 18: 667-674.
Song X, Zhao Y, Song C, Chen M, Huang J, Bao D, Tan Q, Yang R. 2018. Mitogenome types of two Lentinula edodes sensu lato populations in China. Sci Rep. 9: 9421.
Szychowski K A, Skora B, Pomianek T, Gminski J. 2021. Inonotus obliquus: from folk medicine to clinical use. J Trad Complement Med. In press.
Tanaka H, Watanabe K, Ma M, Hirayama M., Kobayashi T, Oyama H. 2009. The effects of g-aminobutyric acid, vinegar, and dried bonito on blood pressure in normotensive and mildly or moderately hypertensive volunteers. J Clinic Biochem Nutr, 45: 93-100.
Tanaka T, Onuma H, Shigihara T, Kimura E, Fukuta Y, Shirasaka N, Moriyama T, Homma Y. 2019. Anti-osteoporotic effects of syringic acid and vanilic acid in the extracts of waste beds after mushroom cultivation. J Biosc Bioengineer. 128: 622-629.
Tura D, Zmitrovich I V, Wasser S P, Spirin W A, Nevo E. 2011. Biodiversity of the Heterobasidiomycetes and non-gilled Hymenomycetes (former Aphyllophorales) of Israel.
Vasantha Rupasinghe H P. 2020. Special Issue “Flavonoids and their disease prevention and treatment potential”: Recent advances and future perspectives. Molecules. 25: 4746.
Vetvicka V, Vannucci L, Sima P, Richter J. 2019. Beta glucan: supplement or drug? from laboratory to clinical trials. Molecules. 24: 1251.
Wang G H, Wang L H, Wang C, Le-Hong Q. 2018b. Spore powder of Ganoderma lucidum for the treatment of Alzheimer disease: a pilot study. Medicine (Baltimore). 97: e0636.
Wang J, Hu S, Nie S. Yu Q. Xie M. 2016. Reviews on mechanisms of in vitro antioxidant activity of polysaccharides. Oxidat Med Cellular Longev. 5692852.
Wang T. He H, Liu X, Liu C, Liang Y. Mei Y. 2019. Mycelial polysaccharides of Lentinus edodes (shiitake mushroom) in submerged culture exert immunoenhancing effect on macrophage cells via MAPK pathway. Int J Biol Macromol. 130: 745-754.
Wang W H, Zhang J, Feng T, Deng J, Lin C, Fan H, Yu W, Bao H Y, Jia W. 2018a. Structural elucidation of a polysaccharide from Flammulina velutipes and its immunomodulation activities on mouse B lymphocytes. Sci Rep. 8: 3120.
Wang Y, Li H, Li Y, Zhao Y, Xiong F. Liu Y, Xue H, Yang Z, Ni S, Sahil A. 2019a. Coriolus versicolor alleviates diabetic cardiomyopathy by inhibiting cardiac fibrosis and NLRP3 inflammasome activation. Phytother Res. 33: 2737-2748.
Wold C W. Gerwick W H, Wangensteen H, Inngjerdingen K T. 2020. Bioactive triterpenoids and water-soluble melanin from Inonotus obliquus (Chaga) with immunomodulatory activity. J Funct Foods. 71: 104025.
Wasser S. P. (ed.). A. R. G. Gantner Verlag K. G., Ruggell, 589 pp.
Wasser S P, Weis A L. 1999. General description of the most important medicinal higher Basidiomycetes mushrooms. Int J Med Mushrooms. 1: 351-370.
Wasser S P. Didukh MYa, de A. Amazonas M A, Nevo E, Stamets P, da Eira A. F. 2002. Is a widely cultivated culinary-medicinal royal sun Agaricus (the Himematsutake mushroom) indeed Agaricus blazei Murrill? Int J Med Mushrooms. 4: 267-290.
Wasser S P. Didukh MYa, de Amazonas M A, Nevo E, Stamets P, da Eira A F. 2005. Is a widely cultivated culinary-medicinal royal sun Agaricus (champignon do Brazil, or the himematsutake mushroom) Agaricus brasiliensis S. Wasser et al. indeed a synonym of A. subrufescens Peck? Int J Med Mushrooms. 7(3): 507-511.
Wasser S P. 2017. Medicinal mushrooms in human clinical studies. Part I. Anticancer, oncoimmunological, and immunomodulatory activities: a review. Int J Med Mushrooms. 19: 279-317.
Wasser S P. 2010. Medicinal mushroom science: history, current status, future trends, and unsolved problems. Int J Med Mushrooms. 12: 1-16.
Wasser S P. 2011. Medicinal Mushrooms. A collection of selected publications (in honor of his 65th birthday). Vol.II. Ukrainian Natinal Academy of Science Publisher, Kiev, 421 pp.
Wei Q, Zhan Y, Chen C, Xie B, Fang T, Ravishankar S, Jiang Y. 2019. Assessment of antioxidant and antidiabetic properties of Agaricus blazei Murill extracts. Food Sci Nutr. 8: 332-339.
WHO. 2020. Healthy diet. World Health Organization. https://www.who.int/news-room/fact-sheets/detail/healthy-diet.
Wu M, Luo X, Xu X, Wei W, Yu M, Jiang N, Ye L, Yang Z, Fei X. 2014. Antioxidant and immunomodulatory activities of a polysaccharide from Flammulina velutipes. J Tradit Chin Med. 34: 733-40.
Wu Y, Choi M, Li J, Yang H, Shin H. 2016. Mushroom cosmetics: the present and future. Cosmetics. 3: 22.
Wu Y, Wei Z, Zhang F, Linhardt R J, Sun P, Zhang A. 2019. Structure, bioactivities and applications of the polysaccharides from Tremella fuciformis mushroom: a review. Int J Biol Macromol. 121: 1005-1010.
Xiaokang W, Lyng J G, Brunton N P, Cody L, Jacquier J, Harrison S M, Papoutsis K. 2020. Monitoring the effect of different microwave extraction parameters on the recovery of polyphenols from shiitake mushrooms: comparison with hot-water and organic-solvent extractions. Biotechnol Reports. 27: e00504. a.
Xiaokang W, Brunton N P, Lyng J G, Harrison S M, Carpes S T, Papoutsis K. 2020. Volatile and non-volatile compounds of shiitake mushrooms treated with pulsed light after twenty-four-hour storage at different conditions. Food Biosci. 36: 100619. b.
Zhang J, Ren A, Chen H, Zhao M, Shi L, Chen M, Wang H, Feng Z. 2015. Transcriptome Analysis and Its Application in Identifying Genes Associated with Fruiting Body Development in Basidiomycete Hypsizygus marmoreus. PLOS ONE. 10: e0123025.
Zhao M, Zhang J, Chen Q, Wu X, Gao W, Deng W, Huang C. 2016. The famous cultivated mushroom Bailinggu is a separate species of the Pleurotus eryngii species complex. Sci Reports. 6: 33066.
Zhong Y, Shahidi F. 2015. Methods for the assessment of antioxidant activity in foods. Handbook of Antioxidants for Food Preservation. Woodhead Publishing Series in Food Science, Technology and Nutrition. Pages 287-333.
Zieba P, Kała K, Włodarczyk A, Szewczyk A, Kunicki E. Sekara A. Muszynska B. 2020. Selenium and zinc biofortification of Pleurotus eryngii mycelium and fruiting bodies as a tool for controlling their biological activity. Molecules. 25: 889.

1. A new and distinct variety of higher Basidiomycetes mushroom selected from:
(i) Coprinus comatus (O.F. Müll.) Pers. deposited under the Budapest Treaty with the Centraalbureau voor Schimmelcultures (CBS) under Accession No. CBS 146994 (hereinafter Coprinus comatus CBS 146994);
(ii) Flammulina velutipes (Curtis) Singer deposited under the Budapest Treaty with the CBS under Accession No. CBS 146995 (hereinafter Flammulina velutipes CBS 146995);
(iii) Ganoderma lucidum (Curtis: Fr.) P. Karst. deposited under the Budapest Treaty with the CBS under Accession No. CBS 146996 (hereinafter Ganoderma lucidum CBS 146996);
(iv) Grifola frondosa (Dicks.) Gray deposited under the Budapest Treaty with the CBS under Accession No. CBS 146997 (hereinafter Grifola frondosa CBS 146997);
(v) Hericium erinaceus (Bull.: Fr.) Pers. deposited under the Budapest Treaty with the CBS under Accession No. CBS 146998 (hereinafter Hericium erinaceus CBS 146998);
(vi) Pleurotus ostreatus (Jacq.: Fr.) Kummer deposited under the Budapest Treaty with the CBS under Accession No. CBS 146999 (hereinafter Pleurotus ostreatus CBS 146999); and
(vii) Trametes versicolor (L.: Fr.) Lloyd. deposited under the Budapest Treaty with the CBS under Accession No. CBS 147000 (hereinafter Trametes versicolor CBS 147000).

2. A biomass of the higher Basidiomycetes mushroom of claim 1.

3. The biomass of claim 2, in the form of a powder.

4. An extract obtained from a higher Basidiomycetes mushroom or a combination thereof, wherein at least one of said higher Basidiomycetes mushrooms is the higher Basidiomycetes mushroom of claim 1.

5. The extract of claim 4, wherein at least another one of said higher Basidiomycetes mushrooms is selected from Agaricus brasiliensis, e.g., Agaricus brasiliensis Wasser et al., Auricularia aricula-judae, e.g., Auricularia aricula-judae (Bull.) J. Schröt., Cordyceps militaris, e.g., Cordyceps militaris (L.) Link, Ganoderma tsugae, e.g., Ganoderma tsugae Murrill, Hypsizygus marmoreus, e.g., Hypsizygus marmoreus (Peck) H. E. Bigelow, Inonotus obliquus, e.g., Inonotus obliquus (Ach. ex Pers.) Pilát, Lentinus edodes, e.g., Lentinus edodes (Berk.) Singer, Pleurotus eryngii, e.g., Pleurotus eryngii (DC: Fr.) Quél., and Tremella fuciformis, e.g., Tremella fuciformis Berk.

6. The extract of claim 4 or 5, obtained by extracting said higher Basidiomycetes mushroom or combination thereof with water at a temperature of about 60-90° C., e.g., at about 80° C., or with an organic solvent such as methanol, ethanol, isopropanol, hexanol, heptane, cyclohexane, methylcyclohexane, dichloromethane, acetonitrile, acetone, methyl ethyl ketone, diethyl ether, methyl-tert-butyl ether, chloroform, tetrahydrofuran, dioxane, phenol, or a mixture thereof.

7. The extract of claim 6, wherein said combination of higher Basidiomycetes mushrooms includes:
(i) Coprinus comatus CBS 146994, Lentinus edodes, and Tremella fuciformis, preferably at about equal weights;
(ii) Flammulina velutipes CBS 146995, Ganoderma lucidum CBS 146996, Lentinus edodes, and Trametes versicolor CBS 147000, preferably at about equal weights;
(iii) Cordyceps militaris, Ganoderma lucidum CBS 146996, and Inonotus obliquus, preferably at a weight ratio of about 1:1:2, respectively;
(iv) Grifola frondosa CBS 146997, Lentinus edodes, Pleurotus eryngii, and Trametes versicolor CBS 147000, preferably at about equal weights;
(v) Auricularia aricula-judae, Ganoderma lucidum CBS 146996, Lentinus edodes, Pleurotus ostreatus CBS 146999, and Trametes versicolor CBS 147000, preferably at a weight ratio of about 1.5:1.5:1.5:4:1.5, respectively;
(vi) Auricularia aricula-judae, Ganoderma lucidum CBS 146996, Lentinus edodes, Pleurotus ostreatus CBS 146999, and Trametes versicolor CBS 147000, preferably at a weight ratio of about 2:2:1.5:3:1.5, respectively;
(vii) Flammulina velutipes CBS 146995, Ganoderma lucidum CBS 146996, Hypsizygus marmoreus, Lentinus edodes, and Trametes versicolor CBS 147000, preferably at about equal weights;
(viii) Cordyceps militaris, Ganoderma lucidum CBS 146996, and Inonotus obliquus, preferably at weight ratio of about 3:4:3, respectively;
(ix) Cordyceps militaris Ganoderma lucidum CBS 146996, and Hericium erinaceus CBS 146998, preferably at a weight ratio of about 3:3:4, respectively;
(x) Cordyceps militaris, Ganoderma lucidum CBS 146996, and Ganoderma tsugae, preferably at a weight ratio of about 2:1:1, respectively;
(xi) Ganoderma lucidum CBS 146996, and of Hericium erinaceus CBS 146998, preferably at a weight ratio of about 1:3, respectively;
(xii) Agaricus brasiliensis, Coprinus comatus CBS 146994, Cordyceps militaris, Lentinus edodes, and Pleurotus ostreatus CBS 146999, preferably at a weight ratio of about 1.5:1.5:1.5:4:1.5, respectively;
(xiii) Agaricus brasiliensis (20%), Coprinus comatus CBS 146994 (40%), Ganoderma lucidum CBS 146996 (20%), and Tremella fuciformis (20%), preferably at a weight ratio of about 1:2:1:1, respectively;
(xiv) Agaricus brasiliensis, Cordyceps militaris, Ganoderma lucidum CBS 146996, Grifola frondosa CBS 146997, and Tremella fuciformis, preferably at about equal weights; or
(xv) Trametes versicolor CBS 147000, Cordyceps militaris, Flammulina velutipes CBS 146995, Ganoderma lucidum CBS 146996, and Lentinus edodes, preferably at about equal weights,
and said extract is obtained by extracting said combination of higher Basidiomycetes mushrooms with water.

8. A composition comprising an extract according to any one of claims 4-7, or a combination thereof, preferably wherein said extract is obtained by extracting said higher Basidiomycetes mushroom or combination thereof with water at a temperature of about 60-90° C. or with an alcohol.

9. The composition of claim 8, comprising an extract obtained by extracting with water a combination of:
(i) Coprinus comatus CBS 146994, Lentinus edodes, and Tremella fuciformis, preferably at about equal weights;
(ii) Flammulina velutipes CBS 146995, Ganoderma lucidum CBS 146996, Lentinus edodes, and Trametes versicolor CBS 147000, preferably at about equal weights;
(iii) Cordyceps militaris, Ganoderma lucidum CBS 146996, and Inonotus obliquus, preferably at a weight ratio of about 1:1:2, respectively;
(iv) Grifola frondosa CBS 146997, Lentinus edodes, Pleurotus eryngii, and Trametes versicolor CBS 147000, preferably at about equal weights;
(v) Auricularia aricula-judae, Ganoderma lucidum CBS 146996, Lentinus edodes, Pleurotus ostreatus CBS 146999, and Trametes versicolor CBS 147000, preferably at a weight ratio of about 1.5:1.5:1.5:4:1.5, respectively;
(vi) Auricularia aricula-judae, Ganoderma lucidum CBS 146996, Lentinus edodes, Pleurotus ostreatus CBS 146999, and Trametes versicolor CBS 147000, preferably at a weight ratio of about 2:2:1.5:3:1.5, respectively;
(vii) Flammulina velutipes CBS 146995, Ganoderma lucidum CBS 146996, Hypsizygus marmoreus, Lentinus edodes, and Trametes versicolor CBS 147000, preferably at about equal weights;
(viii) Cordyceps militaris, Ganoderma lucidum CBS 146996, and Inonotus obliquus, preferably at a weight ratio of about 3:4:3, respectively;
(ix) Cordyceps militaris Ganoderma lucidum CBS 146996, and Hericium erinaceus CBS 146998, preferably at a weight ratio of about 3:3:4, respectively;
(x) Cordyceps militaris, Ganoderma lucidum CBS 146996, and Ganoderma tsugae, preferably at a weight ratio of about 2:1:1, respectively;
(xi) Ganoderma lucidum CBS 146996, and of Hericium erinaceus CBS 146998, preferably at a weight ratio of about 1:3, respectively;
(xii) Agaricus brasiliensis, Coprinus comatus CBS 146994, Cordyceps militaris, Lentinus edodes, and Pleurotus ostreatus CBS 146999, preferably at a weight ratio of about 1.5:1.5:1.5:4:1.5, respectively;
(xiii) Agaricus brasiliensis (20%), Coprinus comatus CBS 146994 (40%), Ganoderma lucidum CBS 146996 (20%), and Tremella fuciformis (20%), preferably at a weight ratio of about 1:2:1:1, respectively;
(xiv) Agaricus brasiliensis, Cordyceps militaris, Ganoderma lucidum CBS 146996, Grifola frondosa CBS 146997, and Tremella fuciformis, preferably at about equal weights; or
(Xv) Trametes versicolor CBS 147000, Cordyceps militaris, Flammulina velutipes CBS 146995, Ganoderma lucidum CBS 146996, and Lentinus edodes, preferably at about equal weights.

10. The composition of claim 8, comprising a combination of (i) an extract obtained by extracting with water a combination of Trametes versicolor CBS 147000, Cordyceps militaris, Flammulina velutipes CBS 146995, Ganoderma lucidum CBS 146996, and Lentinus edodes, e.g., at about equal weights; and (ii) an extract obtained by extracting Ganoderma lucidum CBS 146996 with an alcohol, e.g., ethanol, at a ratio of about 9:1, respectively, by volume.

11. The composition of any one of claims 8-10, further comprising an active agent.

12. The composition of claim 11, wherein said active agent is a cannabinoid, a combination thereof, or a Cannabis plant extract comprising said cannabinoid or combination thereof.

13. The composition of claim 12, wherein said cannabinoid is selected from cannabidiol (CBD), cannabidiolic acid (CBDA), cannabidivarin (CBDV), cannabidivarinic acid (CBDVA), cannabidiol monomethyl ether (CBDM), cannabidiol-C4 (CBD-C4), cannabidiorcol (CBD-C1), Δ⁹-tetrahydrocannabinol (Δ⁹-THC), Δ⁹-tetrahydrocannabinolic acid (Δ⁹-THCA), Δ⁹-tetrahydrocannabivarin (Δ⁹-THCV), Δ⁹-THCVA, Δ⁸-THC, Δ⁸-THCA, Δ⁸-THCV, Δ⁸-THCVA, iso-tetrahydrocannabinol-type (iso-THC), cannabinol (CBN), cannabinolic acid (CBNA), cannabinol-C4 (CBN-C4), cannabinol-C2 (CBN-C2), cannabiorcol (CBN-C1), cannabinol methyl ether (CBNM), cannabinodiol (CBND), cannabigerol (CBG), cannabigerovarin (CBGV), cannabigerolic acid (CBGA), cannabigerovarinic acid (CBGVA), cannabigerol monomethyl ether (CBGM), cannabigerolic acid monomethyl ether (CBGAM), cannabichromene (CBC), cannabichromenic acid (CBCA), cannabichromevarin (CBCV), cannabichromevarinic acid (CBCVA), cannabichromanon (CBCN), cannabicyclol (CBL), cannabicyclolic acid (CBLA), cannabicyclovarin (CBLV), cannabivarin (CBV), cannabivarinic acid (CBVA), cannabielsoin (CBE), cannabielsoic acid A (CBEA-A), cannabielsoic acid B (CBEA-B), cannabitriol (CBT), cannabitriolvarin (CBTV), ethoxy-cannabitiolvarin (CBTVE), cannabifuran (CBF), dehydrocannabifuran (DCBF), cannabiripsol (CBR), an enantiomer, diastereomer, or pharmaceutically acceptable salt thereof, or a mixture thereof.

14. The composition of claim 13, wherein said cannabinoid is CBD, or an enantiomer, diastereomer, or a mixture thereof.

15. A nutraceutical composition according to any one of claims 8-14, optionally further comprising a nutraceutically acceptable carrier.

16. The nutraceutical composition of claim 15, in the form of a liquid, semi-solid, or solid.

17. The nutraceutical composition of claim 16, formulated as tablets, caplets, pills, hard or soft capsules, troches, lozenges, or dispersible powder or granules.

18. A drink, beverage, or a food supplement, comprising a composition according to any one of claims 8-14.

19. A cosmetic composition according to any one of claims 8-14, optionally further comprising a cosmetically acceptable carrier.

20. The cosmetic composition of claim 19, formulated as an ointment, salve, cream, paste, lotion, or oil-in-water emulsion.

21. A method of providing amino acids, phenolic compounds, flavonoid compounds, and/or antioxidant substances to a subject, said method comprising administering to said subject a nutraceutical composition according to any one of claims 15-17.

22. Use of a nutraceutical composition according to any one of claims 15-17 as a dietary supplement rich in amino acids, phenolic compounds, flavonoid compounds, and/or antioxidant substances.
